(19) United States
(12) Patent Application Publication
(10) Pub. No.: US 2025/0145622 A1
(43) Pub. Date: May 8, 2025

(54) SOLID FORMS OF A TRIAZINE DERIVATIVE AS CBL-B MODULATOR

(71) Applicant: HOTSPOT THERAPEUTICS, INC., Boston, MA (US)

(72) Inventors: Jon P. LAWSON, Wildwood, MO (US); Ken CARSON, Princeton, NJ (US); Yingzhi BI, Plainsboro, NJ (US)

(21) Appl. No.: 19/016,543

(22) Filed: Jan. 10, 2025

Related U.S. Application Data
(63) Continuation of application No. PCT/US2023/070010, filed on Jul. 12, 2023.
(60) Provisional application No. 63/388,342, filed on Jul. 12, 2022.

(51) Int. Cl. C07D 471/04; C07C 59/265

(52) U.S. Cl. CPC C07D 471/04; C07C 59/265; C07B 2200/13

(57) ABSTRACT

The present disclosure provides, in part, provides crystalline forms of the compound of formula (I). Also provided are pharmaceutical compositions including a crystalline form of the compound of formula (I), as well as methods of treating various condition, diseases, and disorders using the crystalline forms of the compound of formula (I) and pharmaceutical compositions.

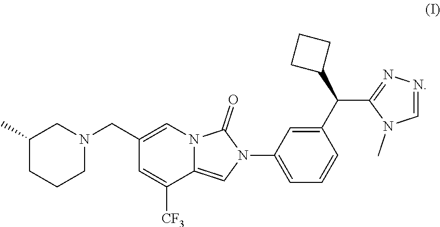 [SMILES: C[C@H]1CCCN(Cc2cc(C(F)(F)F)c3cn(-c4cccc([C@H](c5nncn5C)C5CCC5)c4)c(=O)n3c2)C1]

### CROSS-REFERENCE TO RELATED APPLICATIONS

This application is a continuation of International (PCT) Patent Application No. PCT/US2023/070010, filed on Jul. 12, 2023, which claims the benefit of, and priority to, U.S. Patent Application No. 63/388,342, filed on Jul. 12, 2022, the content of each of which is hereby incorporated by reference in its entirety.

### BACKGROUND

Cbl-b is a E3 ubiquitin-protein ligase that functions as a negative regulator of T-cell activation. Modulation of Cbl-b has been shown to be a therapeutic target for a variety of diseases and disorders. There remains an unmet need to develop solid forms (e.g., crystalline forms) of compounds that inhibit Cbl-b that are suitable for administration to patients with conditions, diseases, or disorders, such as cancer.

### SUMMARY

In one aspect, provided herein are crystalline forms (e.g., anhydrous crystalline forms, crystalline solvates, and crystalline salts) of a compound of formula (I)

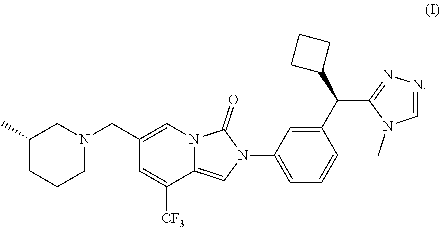 [SMILES: C[C@H]1CCCN(Cc2cc(C(F)(F)F)c3cn(-c4cccc([C@H](c5nncn5C)C5CCC5)c4)c(=O)n3c2)C1]

In various embodiments, a crystalline form of a compound of formula (I):

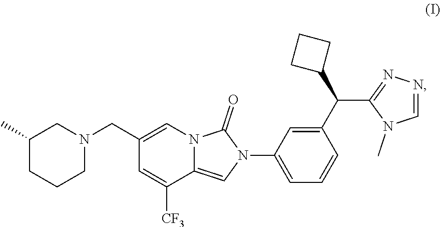 [SMILES: C[C@H]1CCCN(Cc2cc(C(F)(F)F)c3cn(-c4cccc([C@H](c5nncn5C)C5CCC5)c4)c(=O)n3c2)C1]

wherein the crystalline form is characterized by an X-ray powder diffraction pattern comprising one or more peaks selected from 6.0°±0.2°, 8.6°±0.2°, 14.3°±0.2°, and 16.3°±0.2°2-theta.

In various embodiments, a crystalline solvate of a compound of formula (I):

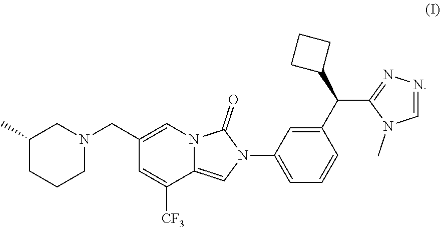 [SMILES: C[C@H]1CCCN(Cc2cc(C(F)(F)F)c3cn(-c4cccc([C@H](c5nncn5C)C5CCC5)c4)c(=O)n3c2)C1]

In various embodiments, a crystalline acetone solvate of a compound of formula (I):

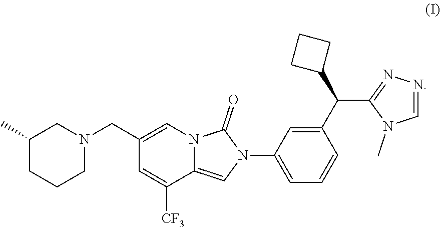 [SMILES: C[C@H]1CCCN(Cc2cc(C(F)(F)F)c3cn(-c4cccc([C@H](c5nncn5C)C5CCC5)c4)c(=O)n3c2)C1]

In various embodiments, a crystalline p-dioxane solvate of a compound of formula (I):

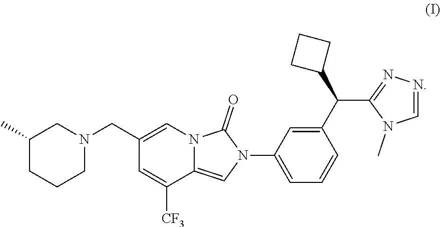 [SMILES: C[C@H]1CCCN(Cc2cc(C(F)(F)F)c3cn(-c4cccc([C@H](c5nncn5C)C5CCC5)c4)c(=O)n3c2)C1]

In various embodiments, a crystalline tetrahydrofuran solvate of a compound of formula (I):

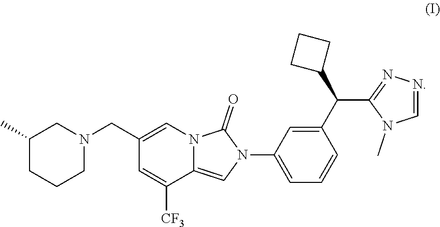 [SMILES: C[C@H]1CCCN(Cc2cc(C(F)(F)F)c3cn(-c4cccc([C@H](c5nncn5C)C5CCC5)c4)c(=O)n3c2)C1]

In various embodiments, a crystalline citrate salt of a compound of formula (I):

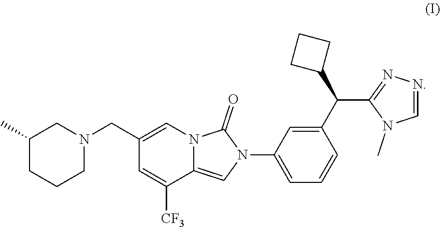 [SMILES: C[C@H]1CCCN(Cc2cc(C(F)(F)F)c3cn(-c4cccc([C@H](c5nncn5C)C5CCC5)c4)c(=O)n3c2)C1]

In another aspect, provided herein are pharmaceutical compositions comprising a crystalline form of the compound of formula (I) described herein and a pharmaceutically acceptable excipient.

In another aspect, provided herein are crystalline forms of the compound of formula (I) (e.g., anhydrous crystalline forms, crystalline solvates, and crystalline salts) and pharmaceutical compositions which are useful for the treatment of the various conditions, diseases, and disorders described herein in a subject in need thereof. In certain embodiments, the condition, disease, or disorder is associated with cell proliferation. In certain embodiments, the condition, disease, or disorder associated with cell proliferation is hyperplasia or a cancer. In certain embodiments, the cancer is a hematologic cancer (e.g., lymphoma, leukemia, and myeloma). In certain embodiments, the cancer is a non-hematologic cancer (e.g., a carcinoma or a sarcoma). In certain embodiments, administration of a crystalline form of the compound of formula (I) or pharmaceutical composition described herein results in the subject exhibiting one or more of the following: increased T-cell activation, increased T-cell proliferation, decreased T-cell exhaustion, decreased T-cell anergy, and decreased T-cell tolerance. In certain embodiments, increased T-cell activation comprises increased production of a cytokines. In certain embodiments, administration of a crystalline form of the compound of formula (I) or pharmaceutical composition described herein results in the subject exhibiting increased NK-cell activation. In certain embodiments, increased NK-cell activation comprises increased production of cytokines.

### BRIEF DESCRIPTION OF THE DRAWINGS

FIG. 1 is an exemplary X-ray powder diffraction (XRPD) pattern of the anhydrous crystalline form of the compound of formula (I), as further described in Example 2.

FIG. 2 is an atomic displacement ellipsoid diagram for the anhydrous crystalline form of the compound of formula (I), as further described in Example 2.

FIG. 3 is an exemplary proton nuclear magnetic resonance (¹H NMR) spectrum of the anhydrous crystalline form of the compound of formula (I) dissolved in dimethylsulfoxide-d₆ containing trimethylsilane, as further described in Example 2.

FIG. 4 shows exemplary differential scanning calorimetry (DSC) and thermogravimetric analysis (TGA) thermograms for the anhydrous crystalline form of the compound of formula (I), as further described in Example 2.

FIG. 5 is an exemplary water sorption isotherm of the anhydrous crystalline form of the compound of formula (I), as further described in Example 2.

FIG. 6 is an exemplary XRPD pattern of the crystalline acetone solvate of the compound of formula (I), as further described in Example 3.

FIG. 7 is an exemplary XRPD indexing solution for the crystalline acetone solvate of the compound of formula (I), as further described in Example 3.

FIG. 8 is an exemplary ¹H NMR spectrum of the crystalline acetone solvate of the compound of formula (I) dissolved in dimethylsulfoxide-d₆ containing trimethylsilane, as further described in Example 3.

FIG. 9 shows exemplary DSC and TGA thermograms for the crystalline acetone solvate of the compound of formula (I), as further described in Example 3.

FIG. 10 is an exemplary XRPD pattern of the crystalline p-dioxane solvate of the compound of formula (I), as further described in Example 4.

FIG. 11 is an exemplary XRPD indexing solution for the crystalline p-dioxane solvate of the compound of formula (I), as further described in Example 4.

FIG. 12 is an exemplary ¹H NMR spectrum of the crystalline p-dioxane solvate of the compound of formula (I) dissolved in dimethylsulfoxide-d₆ containing trimethylsilane, as further described in Example 4.

FIG. 13 shows exemplary DSC and TGA thermograms for the crystalline p-dioxane solvate of the compound of formula (I), as further described in Example 4.

FIG. 14 is an exemplary XRPD pattern of the crystalline tetrahydrofuran (THF) solvate of the compound of formula (I), as further described in Example 5.

FIG. 15 is an exemplary XRPD indexing solution for the crystalline THF solvate of the compound of formula (I), as further described in Example 5.

FIG. 16 is an exemplary ¹H NMR spectrum of the crystalline THF solvate of the compound of formula (I) dissolved in dimethylsulfoxide-d₆ containing trimethylsilane, as further described in Example 5.

FIG. 17 shows exemplary DSC and TGA thermograms for the crystalline THF solvate of the compound of formula (I), as further described in Example 5.

FIG. 18 is an exemplary XRPD pattern of the acetone solvated crystalline citrate salt of the compound of formula (I), as further described in Example 6.

FIG. 19 is an exemplary XRPD indexing solution for the acetone solvated crystalline citrate salt of the compound of formula (I), as further described in Example 6.

FIG. 20 is an exemplary ¹H NMR spectrum of the acetone solvated crystalline citrate salt of the compound of formula (I) dissolved in dimethylsulfoxide-d₆ containing trimethylsilane, as further described in Example 6.

FIG. 21 shows exemplary DSC and TGA thermograms for the acetone solvated crystalline citrate salt of the compound of formula (I), as further described in Example 6.

FIG. 22 is an exemplary XRPD pattern of the acetonitrile solvated crystalline citrate salt of the compound of formula (I), as further described in Example 7.

FIG. 23 is an exemplary XRPD indexing solution for the acetonitrile solvated crystalline citrate salt of the compound of formula (I), as further described in Example 7.

FIG. 24 is an exemplary XRPD pattern of the anhydrous crystalline citrate salt of the compound of formula (I), as further described in Example 8.

FIG. 25 is an exemplary XRPD indexing solution for the anhydrous crystalline citrate salt of the compound of formula (I), as further described in Example 8.

FIG. 26 is an exemplary ¹H NMR spectrum of the anhydrous crystalline citrate salt of the compound of formula (I) dissolved in dimethylsulfoxide-d₆ containing trimethylsilane, as further described in Example 8.

FIG. 27 is an exemplary TGA thermogram for the anhydrous crystalline citrate salt of the compound of formula (I), as further described in Example 8.

FIG. 28 is an exemplary DSC thermogram for the anhydrous crystalline citrate salt of the compound of formula (I), as further described in Example 8.

FIG. 29 is an exemplary water sorption isotherm the anhydrous crystalline citrate salt of the compound of formula (I), as further described in Example 8.

FIG. 30 is an overlay of (i) an XRPD pattern for the anhydrous crystalline form of the compound of formula (I) calculated from single crystal X-ray diffraction data (top pattern); and (ii) an experimentally obtained XRPD pattern of the anhydrous crystalline form of the compound of formula (I) (bottom pattern), as further described in Example 2.

FIG. 31 is an overlay of (i) an XRPD pattern for the anhydrous crystalline form of the compound of formula (I) prior to analysis by dynamic vapor sorption (top pattern); and (ii) an XRPD pattern of the amorphous form of the compound of formula (I) after analysis of the anhydrous crystalline form by dynamic vapor sorption (bottom pattern), as further described in Example 8.

### DETAILED DESCRIPTION

As generally described herein, the disclosure provides crystalline forms of a compound of formula (I) (e.g., anhydrous crystalline forms, crystalline solvates, and crystalline salts), pharmaceutical compositions containing the same, and methods of using said crystalline forms and pharmaceutical compositions to treat medical conditions, diseases, and disorders (e.g., conditions associated with cell proliferation (e.g., hyperplasia or a cancer)) in a subject in need thereof.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element. By way of further example “an analogue” means one analogue or more than one analogue.

The term “and/of” is used in this disclosure to mean either “and” or “of” unless indicated otherwise.

It should be understood that the expression “at least one of” includes individually each of the recited objects after the expression and the various combinations of two or more of the recited objects unless otherwise understood from the context and use. The expression “and/of” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a 10%, ±5%, ±3%, ±2%, or ±1% variation from the nominal value unless otherwise indicated or inferred from the context.

Due to sources of experimental variability that are well known to those of ordinary skill in the art, the values of each differential scanning calorimetry (DSC) endotherm or exotherm are typically preceded with the term “about” or proceeded with an appropriate range defining the experimental variability. For purposes of data reported herein, that value is ±10° C. unless otherwise stated. DSC endotherms/exotherms cited herein are generally reported with this variability of ±10° C. unless stated otherwise and are intended to be reported with such a variability whenever disclosed herein whether the word “about” is present or not, unless context dictates otherwise.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. By way of example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, “pharmaceutically acceptable” and “pharmacologically acceptable,” refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, general safety and purity standards as required by FDA Office of Biologics standards.

As used herein, “pharmaceutically acceptable carrier” and “pharmaceutically acceptable excipient,” refer to any and all solvents, dispersion media, coatings, isotonic and absorption delaying agents, and the like, that are compatible with pharmaceutical administration. Pharmaceutical acceptable carriers can include phosphate buffered saline solution, water, emulsions (e.g., such as an oil/water or water/oil emulsions), and various types of wetting agents. The compositions also can include stabilizers and preservatives.

As used herein, “pharmaceutically acceptable salt” refers to any salt of an acidic or a basic group that may be present in a compound of the present disclosure (e.g., the compound of formula (I)), which salt is compatible with pharmaceutical administration. As is known to those of skill in the art, “salts” of the compounds of the present disclosure may be derived from inorganic or organic acids and bases. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound (e.g., a compound of the present invention) is an amount sufficient to provide a therapeutic benefit in the treatment of a disease, disorder, or condition, or to delay or minimize one or more symptoms associated with the disease, disorder, or condition. A therapeutically effective amount of a compound (e.g., a compound of the present invention) means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent.

As used herein, “disease,” “disorder,” “condition,” or “illness,” can be used interchangeably unless otherwise underacted or understood from the context, refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, or illness e.g., through administration of the compound of formula (I), or a stereoisomer and/or a pharmaceutically acceptable salt thereof.

As used herein, “administering” means oral administration, administration as a suppository, topical contact, intravenous, parenteral, intraperitoneal, intramuscular, intralesional, intrathecal, intracranial, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

### Solid Forms

The compound of formula (I), also known as 2-(3-((R)-cyclobutyl(4-methyl-4H-1,2,4-triazol-3-yl)methyl)phenyl)-6-(((S)-3-methylpiperidin-1-yl)methyl)-8-(trifluoromethyl)imidazo[1,5-a]pyridin-3(2H)-one, is a Cbl-b inhibitor.

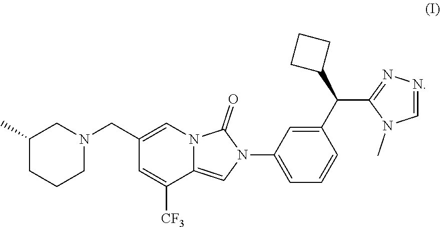 [SMILES: C[C@H]1CCCN(Cc2cc(C(F)(F)F)c3cn(-c4cccc([C@H](c5nncn5C)C5CCC5)c4)c(=O)n3c2)C1]

In one aspect, provided herein are solid forms of the compound of formula (I).

In various embodiments, the solid form of the compound of formula (I) is a crystalline form. In certain embodiments, the compound of formula (I) is present in the crystalline form in its free-base form. In certain embodiments, the compound of formula (I) is present in the crystalline form as a pharmaceutically acceptable salt (e.g., a citrate salt). In certain embodiments, the crystalline form is a solvated crystalline form. In certain embodiments, the crystalline form is an unsolvated crystalline form.

### 1. Crystalline Forms of Compound of Formula (I) Free-Base

In various embodiments, provided herein are crystalline forms of the freebase of the compound of formula (I). In certain embodiments, the crystalline form of the freebase of the compound of formula (I) is an anhydrous crystalline form. In certain embodiments, the crystalline form of the freebase of the compound of formula (I) is a solvated crystalline form. In certain embodiments, the crystalline form of the freebase of the compound of formula (I) is an unsolvated crystalline form.

### (i) Anhydrous Crystal Form

In various embodiments, provided herein is a crystalline form of a compound of formula (I)

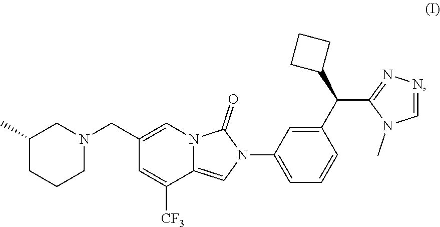 [SMILES: C[C@H]1CCCN(Cc2cc(C(F)(F)F)c3cn(-c4cccc([C@H](c5nncn5C)C5CCC5)c4)c(=O)n3c2)C1]

wherein the crystalline form is characterized by an X-ray powder diffraction (XRPD) pattern comprising one or more peaks selected from 6.0°±0.2°, 8.6°±0.2°, 14.3°±0.2°, and 16.3°±0.2°2-theta.

In certain embodiments, the crystalline form is characterized by an XRPD pattern comprising peaks at 6.0°±0.2°, 8.6°±0.2°, 14.3°±0.2°, and 16.3°±0.2°2-theta.

In certain embodiments, the XRPD pattern further comprises one or more peaks selected from 15.6°±0.2°, 17.4°±0.2°, 18.2°±0.2°, 19.9°±0.2°, 20.4°±0.20, and 21.5°±0.20 2-theta.

In certain embodiments, the XRPD pattern further comprises peaks at 15.6°±0.2°, 17.4°±0.2°, 18.2°±0.2°, 19.9°±0.2°, 20.4°±0.20, and 21.5°±0.20 2-theta.

In certain embodiments, the crystalline form is characterized by an XRPD pattern comprising one or more peaks selected from 6.0°±0.2°, 8.6°±0.2°, 14.3°±0.2°, 15.6°±0.2°, 16.3°±0.2°, 17.4°±0.2°, 18.2°±0.2°, 19.9°±0.2°, 20.4°±0.20, and 21.5°±0.20 2-theta.

In certain embodiments, the crystalline form is characterized by an XRPD pattern comprising peaks at 6.0°±0.2°, 8.6°±0.2°, 14.3°±0.2°, 15.6°±0.2°, 16.3°±0.2°, 17.4°±0.20, 18.2°±0.2°, 19.9°±0.2°, 20.4°±0.20, and 21.5°±0.20 2-theta.

In certain embodiments, the XRPD pattern further comprises one or more peaks selected from 7.1°±0.2°, 11.7°±0.2°, 12.1°±0.2°, 14.7°±0.2°, 15.1°±0.2°, 18.5°±0.2°, 19.6°±0.2°, 20.6°±0.2°, 20.9°±0.2°, 22.0°±0.2°, 22.3°±0.2°, 22.7°±0.2°, 23.0°±0.2°, 23.2°±0.20, 24.4°±0.2°, 24.8°±0.2°, 25.2°±0.2°, 25.6°±0.2°, 26.1°±0.2°, 26.4°±0.2°, 27.1°±0.20, 27.5°±0.2°, 28.1°±0.2°, 28.5°±0.2°, 28.7°±0.2°, 29.3°±0.2°, 29.8°±0.20, and 30.5°±0.20 2-theta.

In certain embodiments, the XRPD pattern further comprises peaks at 7.1°±0.2°, 11.7°±0.2°, 12.1°±0.2°, 14.7°±0.2°, 15.1°±0.2°, 18.5°±0.2°, 19.6°±0.2°, 20.6°±0.2°, 20.90±0.20, 22.0°±0.2°, 22.3°±0.2°, 22.7°±0.2°, 23.0°±0.2°, 23.2°±0.2°, 24.4°±0.2°, 24.80±0.20, 25.2°±0.2°, 25.6°±0.2°, 26.1°±0.2°, 26.4°±0.2°, 27.1°±0.2°, 27.5°±0.2°, 28.10±0.2°, 28.5°±0.2°, 28.7°±0.2°, 29.3°±0.2°, 29.8°±0.2°, and 30.5°±0.2°2-theta.

In certain embodiments, the crystalline form is characterized by an XRPD pattern comprising one or more peaks selected from 6.0°±0.2°, 7.1°±0.2°, 8.6°±0.2°, 11.7°±0.2° 12.1°±0.2°, 14.3°±0.2°, 14.7°±0.2°, 15.1°±0.2°, 15.6°±0.2°, 16.3°±0.2°, 17.4°±0.20, 18.2°±0.2°, 18.5°±0.2°, 19.6°±0.2°, 19.9°±0.2°, 20.4°±0.2°, 20.6°±0.2°, 20.9°±0.20, 21.5°±0.2°, 22.0°±0.2°, 22.3°±0.2°, 22.7°±0.2°, 23.0°±0.2°, 23.2°±0.2°, 24.4°±0.20, 24.8°±0.2°, 25.2°±0.2°, 25.6°±0.2°, 26.1°±0.2°, 26.4°±0.2°, 27.1°±0.2°, 27.5°±0.20, 28.1°±0.2°, 28.5°±0.2°, 28.7°±0.2°, 29.3°±0.2°, 29.8°±0.20, and 30.5°±0.20 2-theta.

In certain embodiments, the crystalline form is characterized by an XRPD pattern comprising peaks at 6.0°±0.2°, 7.1°±0.2°, 8.6°±0.2°, 11.7°±0.2°, 12.1°±0.2°, 14.3°±0.2°, 14.7°±0.2°, 15.1°±0.2°, 15.6°±0.2°, 16.3°±0.2°, 17.4°±0.2°, 18.2°±0.2°, 18.5°±0.20, 19.6°±0.2°, 19.9°±0.2°, 20.4°±0.2°, 20.6°±0.2°, 20.9°±0.2°, 21.5°±0.2°, 22.0°±0.20, 22.3°±0.2°, 22.7°±0.2°, 23.0°±0.2°, 23.2°±0.2°, 24.4°±0.2°, 24.8°±0.2°, 25.2°±0.20, 25.6°±0.2°, 26.1°±0.2°, 26.4°±0.2°, 27.1°±0.2°, 27.5°±0.2°, 28.1°±0.2°, 28.5°±0.20, 28.7°±0.2°, 29.3°±0.2°, 29.8°±0.2°, and 30.5°±0.2°2-theta.

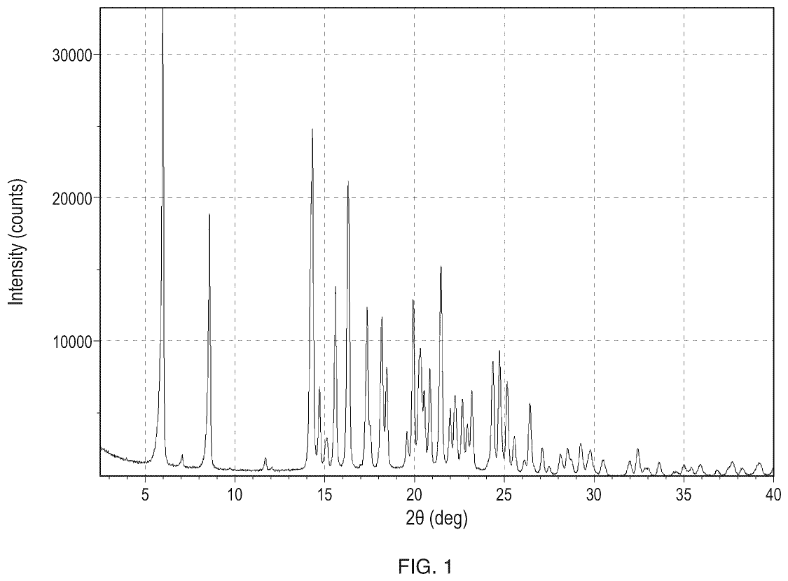

In certain embodiments, the crystalline form is characterized by an XRPD pattern substantially the same as shown in FIG. 1.

In certain embodiments, the crystalline form exists in an orthorhombic crystal system and has a P2₁2₁2₁ space group. In certain embodiments, the crystalline form is characterized by the crystallographic unit cell parameters as set forth in Table 1.

TABLE 1
Unit Cell Parameters of the Anhydrous Crystalline Form

| | | |
|---|---|---|
| Unit cell dimensions | a = 6.37450(10) Å | α = 90° |
| | b = 18.1063(3) Å | β = 90° |
| | c = 24.8603(4) Å | γ = 90° |

| | |
|---|---|
| Volume | 2869.34(8) Å³ |
| Z | 4 |
| Calculated Density | 1.247 g cm⁻³ |

In certain embodiments, the crystalline form dissolved in dimethylsulfoxide-d₆ containing trimethylsilane is characterized by a proton nuclear magnetic resonance (¹H NMR) spectrum having one, two, three, four, five, or more peaks at 8.327, 7.744, 7.741, 7.738, 7.701, 7.699, 7.697, 7.696, 7.687, 7.686, 7.684, 7.682, 7.646, 7.452, 7.439, 7.426, 7.305, 7.199, 7.186. 7.003, 6.539, 6.395, 6.367, 6.354, 6.328, 5.769, 4.571, 4.561, 4.552, 4.500, 4.270, 4.252, 3.430, 3.327, 3.245, 3.227, 3.216, 3.211, 3.198, 2.765, 2.740, 2.725, 2.511, 2.508, 2.505, 2.502, 2.499, 2.081, 2.075, 2.068, 2.063, 1.906, 1.889, 1.872, 1.850, 1.847, 1.837, 1.834, 1.829, 1.823, 1.813, 1.804, 1.800, 1.794, 1.788, 1.786, 1.780, 1.774, 1.769, 1.757, 1.752, 1.732, 1.718, 1.714, 1.700, 1.686, 1.673, 1.669, 1.654, 1.637, 1.633, 1.612, 1.600, 1.583, 1.579, 1.560, 1.554, 1.549, 1.543, 1.484, 1.478, 1.472, 1.463, 1.458, 1.452, 1.443, 1.437, 1.432, 1.417, 1.410, 1.231, 1.144, 0.882, 0.875, 0.860, 0.829, and 0.819 ppm.

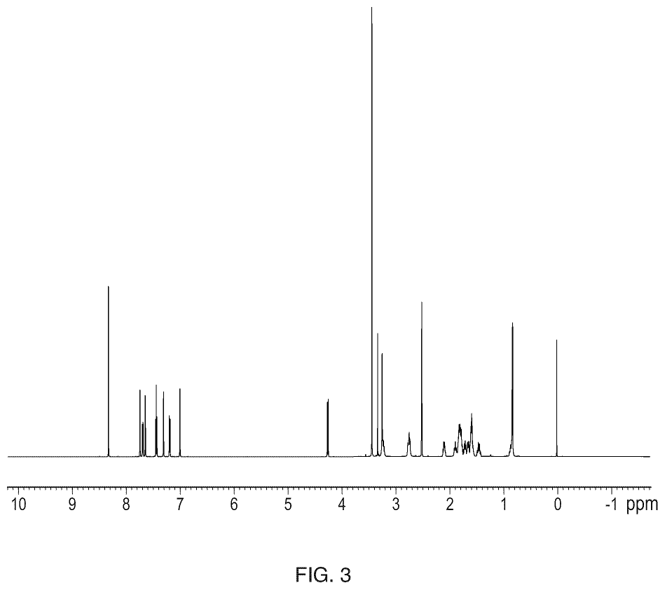

In certain embodiments, the crystalline form dissolved in dimethylsulfoxide-d₆ containing trimethylsilane is characterized by a ¹H NMR spectrum substantially the same as shown in FIG. 3.

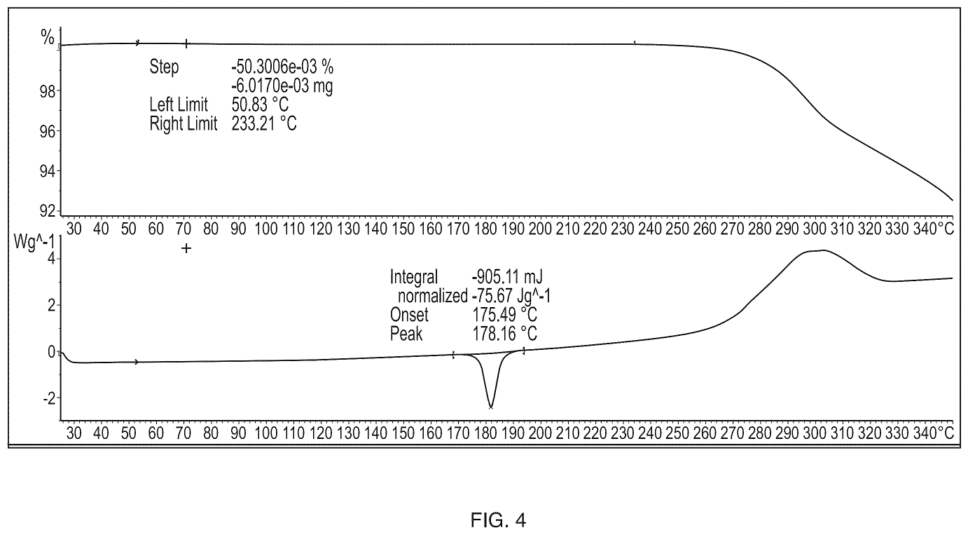

The crystalline form of the compound of formula (I) may also be characterized using a thermoanalytical technique, such as differential scanning calorimetry (DSC). Accordingly, in certain embodiments, the crystalline form is characterized by an endotherm with a peak onset of about 165° C. to about 180° C., as determined by DSC. In certain embodiments, the crystalline form is characterized by an endotherm with a peak onset of about 170° C. to about 180° C., as determined by DSC. In certain embodiments, the crystalline form is characterized by an endotherm with a peak onset of about 175° C., as determined by DSC. In certain embodiments, the crystalline form is characterized by a melting point onset of about 165° C. to about 180° C., as determined by DSC. In certain embodiments, the crystalline form is characterized by a melting point onset of about 170° C. to about 180° C., as determined by DSC. In certain embodiments, the crystalline form is characterized by a melting point onset of about 175° C., as determined by DSC. In certain embodiments, the crystalline form has a DSC thermogram substantially the same as shown in FIG. 4.

The crystalline form may also be characterized according to its mass gain/mass loss as a function of temperature. Accordingly, in certain embodiments, the crystalline form exhibits a reduction in mass, as determined by thermogravimetric analysis (TGA), of from about 0.1% to about 1.6% wt. upon heating to about 230° C. In certain embodiments, the crystalline form exhibits a reduction in mass, as determined by TGA analysis, of less than or equal to about 1.6% wt. upon heating to about 230° C. In certain embodiments, the crystalline form has a TGA thermogram substantially the same as shown in FIG. 4.

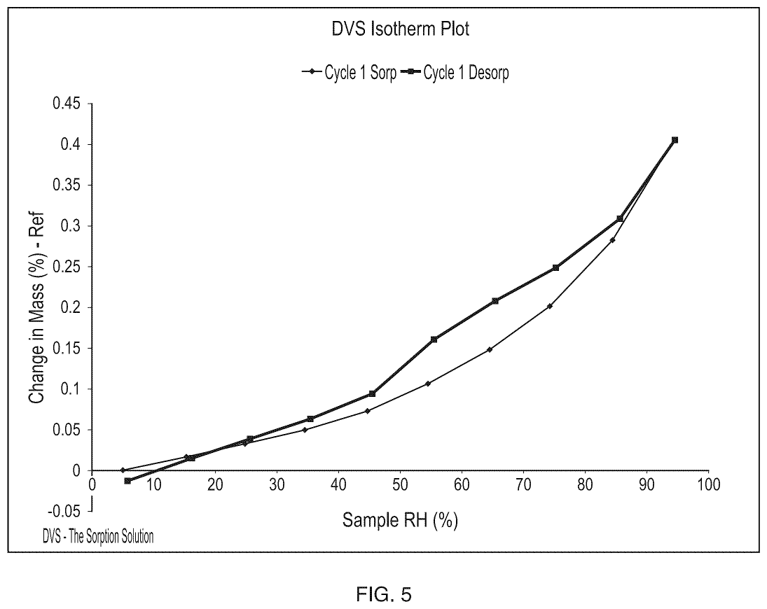

The crystalline form may also be characterized according to its water sorption properties. Accordingly, in certain embodiments, the crystalline form exhibits an increase in mass, as determined by dynamic vapor sorption (DVS), of less than or equal to about 0.4% wt. at a relative humidity of 95% and a temperature of 25° C. In certain embodiments, the crystalline form has a water sorption isotherm, when measured at 25° C., substantially the same as shown in FIG. 5.

In certain embodiments, the crystalline form is an anhydrous crystalline form.

### (ii) Crystalline Acetone Solvate

In various embodiments, provided herein is a crystalline acetone solvate of a compound of formula (I):

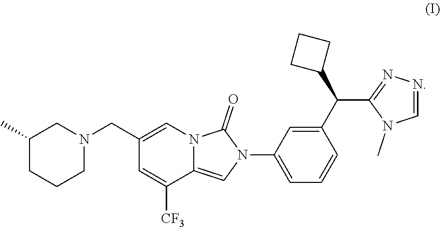 [SMILES: C[C@H]1CCCN(Cc2cc(C(F)(F)F)c3cn(-c4cccc([C@H](c5nncn5C)C5CCC5)c4)c(=O)n3c2)C1]

In certain embodiments, the crystalline acetone solvate is characterized by an XRPD pattern comprising one or more peaks selected from 6.4°±0.2°, 16.2°±0.2°, 17.2°±0.2°, and 22.0°±0.2°2-theta.

In certain embodiments, the crystalline acetone solvate is characterized by an XRPD pattern comprising peaks at 6.4°±0.2°, 16.2°±0.2°, 17.2°±0.2°, and 22.0°±0.2°2-theta.

In certain embodiments, the XRPD pattern further comprises one or more peaks selected from 12.0°±0.2°, 14.3°±0.2°, 15.7°±0.2°, 17.8°±0.2°, 20.2°±0.2°, 21.8°±0.20, and 22.60±0.2°2-theta.

In certain embodiments, the XRPD pattern further comprises peaks at 12.0°±0.20, 14.3°±0.2°, 15.7°±0.2°, 17.8°±0.2°, 20.2°±0.2°, 21.8°±0.20, and 22.6°±0.20 2-theta.

In certain embodiments, the crystalline acetone solvate is characterized by an XRPD pattern comprising one or more peaks selected from 6.4°±0.2°, 12.0°±0.2°, 14.3°±0.2°, 15.7°±0.2°, 16.2°±0.2°, 17.2°±0.2°, 17.8°±0.2°, 20.2°±0.2°, 21.8°±0.2°, 22.0°±0.20, and 22.60±0.2°2-theta.

In certain embodiments, the crystalline acetone solvate is characterized by an XRPD pattern comprising peaks at 6.4°±0.2°, 12.0°±0.2°, 14.3°±0.2°, 15.7°±0.2°, 16.2°±0.2°, 17.2°±0.2°, 17.8°±0.2°, 20.2°±0.2°, 21.8°±0.2°, 22.0°±0.20, and 22.6°±0.20 2-theta.

In certain embodiments, the XRPD pattern further comprises one or more peaks selected from 7.8°±0.2°, 9.8°±0.2°, 10.4°±0.2°, 12.8°±0.2°, 14.1°±0.2°, 15.1°±0.2°, 17.6°±0.20, 18.4°±0.2°, 18.6°±0.2°, 19.2°±0.2°, 20.5°±0.2°, 21.2°±0.2°, 22.8°±0.2°, 23.3°±0.20, 23.6°±0.2°, 24.0°±0.2°, 24.3°±0.2°, 24.9°±0.2°, 25.0°±0.2°, 25.4°±0.2°, 25.8°±0.20, 26.1°±0.2°, 26.3°±0.2°, 27.0°±0.2°, 27.7°±0.2°, 28.3°±0.2°, 28.7°±0.2°, 29.3°±0.20, 29.7°±0.2°, 30.1°±0.2°, 30.6°±0.20, and 31.1°±0.20 2-theta.

In certain embodiments, the XRPD pattern further comprises peaks at 7.8°±0.2°, 9.8°±0.20, 10.4°±0.2°, 12.8°±0.2°, 14.1°±0.2°, 15.1°±0.2°, 17.6°±0.2°, 18.4°±0.2°, 18.60±0.20, 19.2°±0.2°, 20.5°±0.2°, 21.2°±0.2°, 22.8°±0.2°, 23.3°±0.2°, 23.6°±0.2°, 24.00±0.20, 24.3°±0.2°, 24.9°±0.2°, 25.0°±0.2°, 25.4°±0.2°, 25.8°±0.2°, 26.1°±0.2°, 26.30±0.2°, 27.0°±0.2°, 27.7°±0.2°, 28.3°±0.2°, 28.7°±0.2°, 29.3°±0.2°, 29.7°±0.2°, 30.1°±0.2°, 30.6°±0.2°, and 31.1°±0.2°2-theta.

In certain embodiments, the crystalline acetone solvate is characterized by an XRPD pattern comprising one or more peaks selected from 6.4°±0.2°, 7.8°±0.2°, 9.8°±0.2°, 10.4°±0.20, 12.0°±0.2°, 12.8°±0.2°, 14.1°±0.2°, 14.3°±0.2°, 15.1°±0.2°, 15.7°±0.2°, 16.20±0.20, 17.2°±0.2°, 17.6°±0.2°, 17.8°±0.2°, 18.4°±0.2°, 18.6°±0.2°, 19.2°±0.2°, 20.20±0.20, 20.5°±0.2°, 21.2°±0.2°, 21.8°±0.2°, 22.0°±0.2°, 22.6°±0.2°, 22.8°±0.2°, 23.30±0.20, 23.6°±0.2°, 24.0°±0.2°, 24.3°±0.2°, 24.9°±0.2°, 25.0°±0.2°, 25.4°±0.2°, 25.80±0.20, 26.1°±0.2°, 26.3°±0.2°, 27.0°±0.2°, 27.7°±0.2°, 28.3°±0.2°, 28.7°±0.2°, 29.3°±0.2°, 29.7°±0.2°, 30.1°±0.2°, 30.6°±0.2°, and 31.1°±0.2°2-theta.

In certain embodiments, the crystalline acetone solvate is characterized by an XRPD pattern comprising peaks at 6.4°±0.2°, 7.8°±0.2°, 9.8°±0.2°, 10.4°±0.2°, 12.0°±0.2°, 12.8°±0.2°, 14.1°±0.2°, 14.3°±0.2°, 15.1°±0.2°, 15.7°±0.2°, 16.2°±0.2°, 17.2°±0.2°, 17.60±0.20, 17.8°±0.2°, 18.4°±0.2°, 18.6°±0.2°, 19.2°±0.2°, 20.2°±0.2°, 20.5°±0.2°, 21.20±0.20, 21.8°±0.2°, 22.0°±0.2°, 22.6°±0.2°, 22.8°±0.2°, 23.3°±0.2°, 23.6°±0.2°, 24.00±0.20, 24.3°±0.2°, 24.9°±0.2°, 25.0°±0.2°, 25.4°±0.2°, 25.8°±0.2°, 26.1°±0.2°, 26.30±0.2°, 27.0°±0.2°, 27.7°±0.2°, 28.3°±0.2°, 28.7°±0.2°, 29.3°±0.2°, 29.7°±0.2°, 30.1°±0.2°, 30.6°±0.2°, and 31.1°±0.2°2-theta.

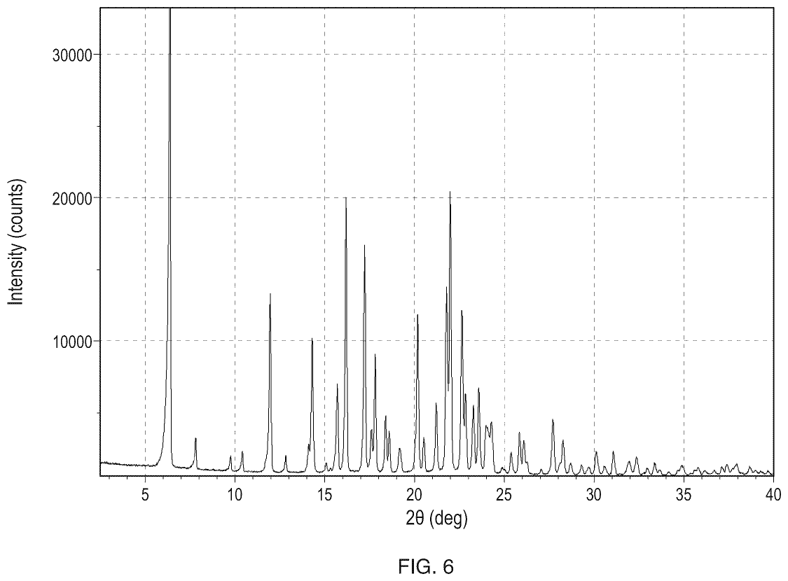

In certain embodiments, the crystalline acetone solvate is characterized by an XRPD pattern substantially the same as shown in FIG. 6.

In certain embodiments, the crystalline acetone solvate exists in an orthorhombic crystal system and has a P2₁2₁2₁ space group. In certain embodiments, the crystalline acetone solvate is characterized by the crystallographic unit cell parameters as set forth in Table 2.

TABLE 2
Unit Cell Parameters of Crystalline Acetone Solvate

| | | |
|---|---|---|
| Unit cell dimensions | a = 6.251 Å | α = 90° |
| | b = 15.082 Å | β = 90° |
| | c = 33.920 Å | γ = 90° |

| | |
|---|---|
| Volume | 3197.9 Å³ |

In certain embodiments, the crystalline acetone solvate dissolved in dimethylsulfoxide-d₆ containing trimethylsilane is characterized by a ¹H NMR spectrum having one, two, three, four, five, or more peaks at 8.323, 7.740, 7.737, 7.734, 7.696, 7.694, 7.693, 7.684, 7.683, 7.681, 7.679, 7.643, 7.451, 7.438, 7.424, 7.303, 7.197, 7.184, 7.002, 4.547, 4.267, 4.249, 3.542, 3.426, 3.400, 3.378, 3.349, 3.320, 3.307, 3.296, 3.265, 3.242, 3.224, 3.213, 3.208, 3.195, 3.181, 3.129, 2.763, 2.738, 2.723, 2.617, 2.614, 2.611, 2.523, 2.520, 2.508, 2.505, 2.502, 2.499, 2.496, 2.481, 2.389, 2.386, 2.109, 2.096, 2.087, 2.081, 2.073, 2.066, 2.060, 1.979, 1.904, 1.888, 1.869, 1.849, 1.846, 1.836, 1.827, 1.822, 1.812, 1.803, 1.799, 1.793, 1.788, 1.785, 1.779, 1.773, 1.767, 1.756, 1.750, 1.730, 1.715, 1.711, 1.698, 1.684, 1.656, 1.638, 1.612, 1.596, 1.580, 1.560, 1.554, 1.478, 1.471, 1.458, 1.452, 1.443, 1.437, 1.432, 1.416, 1.141, 0.883, 0.876, 0.861, 0.830, and 0.820 ppm.

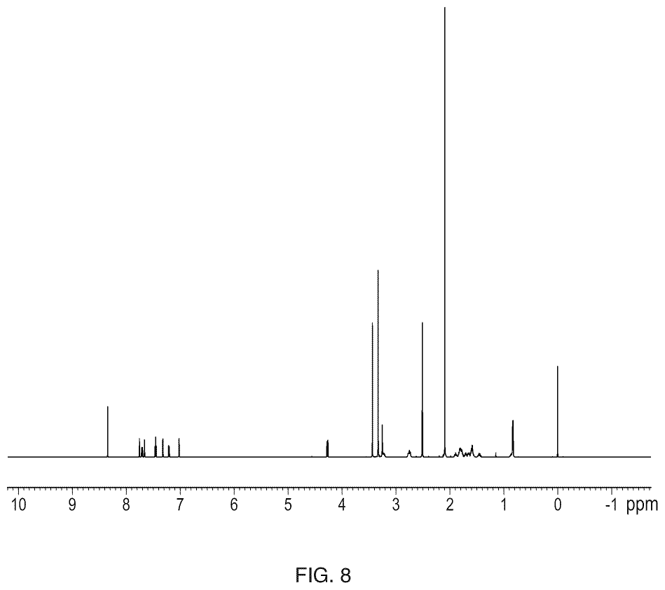

In certain embodiments, the crystalline acetone solvate is characterized by a ¹H NMR spectrum substantially the same as shown in FIG. 8.

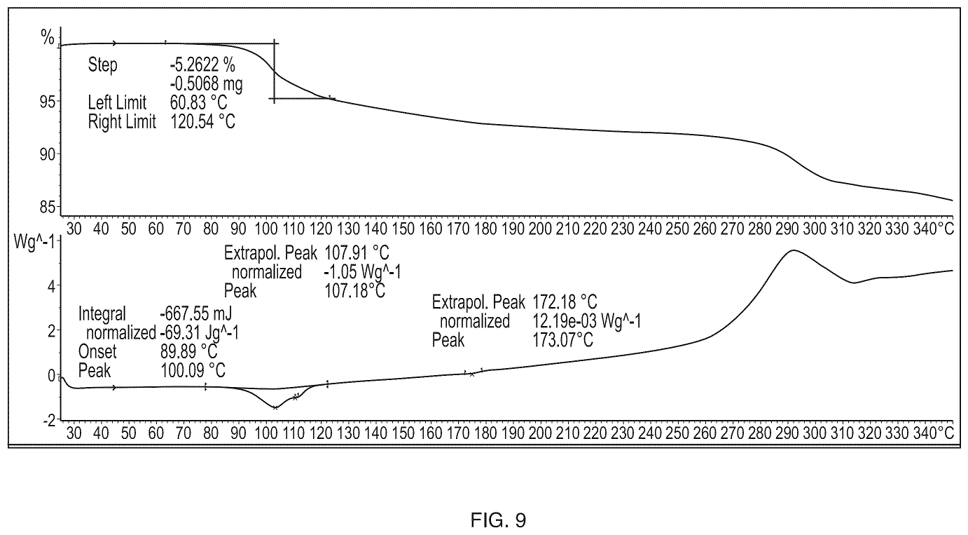

The crystalline acetone solvate of the compound of formula (I) may also be characterized using a thermoanalytical technique, such as DSC. Accordingly, in certain embodiments, the crystalline acetone solvate is characterized by one or more endotherms with peak maxima selected from about 100° C., about 107° C., and about 173° C., as determined by DSC. In certain embodiments, the crystalline acetone solvate is characterized by an endotherm with a peak onset of about 90° C., as determined by DSC. In certain embodiments, the crystalline acetone solvate has a DSC thermogram substantially the same as shown in FIG. 9.

The crystalline acetone solvate may also be characterized according to its mass gain/mass loss as a function of temperature. Accordingly, in certain embodiments, the crystalline acetone solvate exhibits a reduction in mass, as determined by TGA, of less than or equal to about 5.3% wt. upon heating to about 121° C. In certain embodiments, the crystalline acetone solvate has a TGA thermogram substantially the same as shown in FIG. 9.

In certain embodiments, the crystalline acetone solvate is a mono-acetone solvate.

(iii) Crystalline p-Dioxane Solvate

In various embodiments, provided herein is a crystalline p-dioxane solvate of a compound of formula (I):

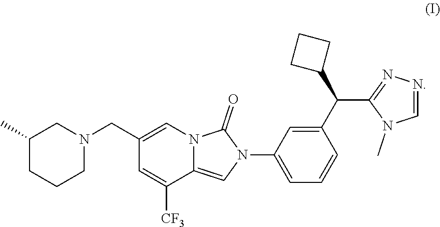 [SMILES: C[C@H]1CCCN(Cc2cc(C(F)(F)F)c3cn(-c4cccc([C@H](c5nncn5C)C5CCC5)c4)c(=O)n3c2)C1]

In certain embodiments, the crystalline p-dioxane solvate is characterized by an XRPD pattern comprising one or more peaks selected from 6.3°±0.2°, 16.0°±0.2°, 17.0°±0.2°, and 21.8°±0.2°2-theta.

In certain embodiments, the crystalline p-dioxane solvate is characterized by an XRPD pattern comprising peaks at 6.3°±0.2°, 16.0°±0.2°, 17.0°±0.2°, and 21.8°±0.2°2-theta.

In certain embodiments, the XRPD pattern further comprises one or more peaks selected from 11.8°±0.2°, 14.2°±0.2°, 17.5°±0.2°, 20.0°±0.2°, and 21.6°±0.2°2-theta.

In certain embodiments, the XRPD pattern further comprises peaks at 11.8°±0.2° 14.2°±0.2°, 17.5°±0.2°, 20.0°±0.2°, and 21.6°±0.2°2-theta.

In certain embodiments, the crystalline p-dioxane solvate is characterized by an XRPD pattern comprising one or more peaks selected from 6.3°±0.2°, 11.8°±0.2°, 14.2°±0.2°, 16.0°±0.2°, 17.0°±0.2°, 17.5°±0.2°, 20.0°±0.2°, 21.6°±0.20, and 21.8°±0.20 2-theta.

In certain embodiments, the crystalline p-dioxane solvate is characterized by an XRPD pattern comprising peaks at 6.3°±0.2°, 11.8°±0.2°, 14.2°±0.2°, 16.0°±0.2°, 17.0°±0.2°, 17.5°±0.2°, 20.0°±0.2°, 21.6°±0.2°, and 21.8°±0.2°2-theta.

In certain embodiments, the XRPD pattern further comprises one or more peaks selected from 5.2°±0.2°, 7.7°±0.2°, 10.3°±0.2°, 12.6°±0.2°, 14.9°±0.2°, 15.4°±0.2°, 15.5°±0.20, 18.1°±0.2°, 18.4°±0.2°, 18.9°±0.2°, 19.1°±0.2°, 19.4°±0.2°, 20.2°±0.2°, 21.0°±0.20, 22.4°±0.2°, 23.0°±0.2°, 23.1°±0.2°, 23.8°±0.2°, 24.0°±0.2°, 24.7°±0.2°, 25.2°±0.20, 25.4°±0.2°, 25.8°±0.2°, 27.3°±0.2°, 27.8°±0.2°, 28.0°±0.2°, 28.3°±0.20, and 28.9°±0.20 2-theta.

In certain embodiments, the XRPD pattern further comprises peaks at 5.2°±0.2°, 7.7° 0.20, 10.3°±0.2°, 12.6°±0.2°, 14.9°±0.2°, 15.4°±0.2°, 15.5°±0.2°, 18.1°±0.2°, 18.40±0.20, 18.9°±0.2°, 19.1°±0.2°, 19.4°±0.2°, 20.2°±0.2°, 21.00 0.2°, 22.4°±0.2°, 23.00±0.20, 23.1°±0.2°, 23.8°±0.2°, 24.0°±0.2°, 24.7°±0.2°, 25.2°±0.2°, 25.4°±0.2°, 25.80±0.20, 27.3°±0.2°, 27.8°±0.2°, 28.0°±0.2°, 28.3°±0.20, and 28.9°±0.20 2-theta.

In certain embodiments, the crystalline p-dioxane solvate is characterized by an XRPD pattern comprising one or more peaks selected from 5.2°±0.2°, 6.3°±0.2°, 7.7°±0.2°, 10.3°±0.20, 11.8°±0.2°, 12.6°±0.2°, 14.2°±0.2°, 14.9°±0.2°, 15.4°±0.2°, 15.5°±0.2°, 16.00±0.20, 17.0°±0.2°, 17.5°±0.2°, 18.1°±0.2°, 18.4°±0.2°, 18.9°±0.2°, 19.1°±0.2°, 19.40±0.20, 20.0°±0.2°, 20.2°±0.2°, 21.0°±0.2°, 21.6°±0.2°, 21.8°±0.2°, 22.4°±0.2°, 23.00±0.20, 23.1°±0.2°, 23.8°±0.2°, 24.0°±0.2°, 24.7°±0.2°, 25.2°±0.2°, 25.4°±0.2°, 25.80±0.20, 27.3°±0.2°, 27.8°±0.2°, 28.0°±0.2°, 28.3°±0.20, and 28.9°±0.20 2-theta.

In certain embodiments, the crystalline p-dioxane solvate is characterized by an XRPD pattern comprising peaks at 5.2°±0.2°, 6.3°±0.2°, 7.7°±0.2°, 10.3°±0.2°, 11.8°±0.2°, 12.6°±0.2°, 14.2°±0.2°, 14.9°±0.2°, 15.4°±0.2°, 15.5°±0.2°, 16.0°±0.2°, 17.0°±0.2°, 17.50±0.20, 18.1°±0.2°, 18.4°±0.2°, 18.9°±0.2°, 19.1°±0.2°, 19.4°±0.2°, 20.0°±0.2°, 20.20±0.20, 21.0°±0.2°, 21.6°±0.2°, 21.8°±0.2°, 22.4°±0.2°, 23.0°±0.2°, 23.1°±0.2°, 23.80±0.20, 24.0°±0.2°, 24.7°±0.2°, 25.2°±0.2°, 25.4°±0.2°, 25.8°±0.2°, 27.3°±0.2°, 27.80±0.2°, 28.0°±0.2°, 28.3°±0.2°, and 28.9°±0.2°2-theta.

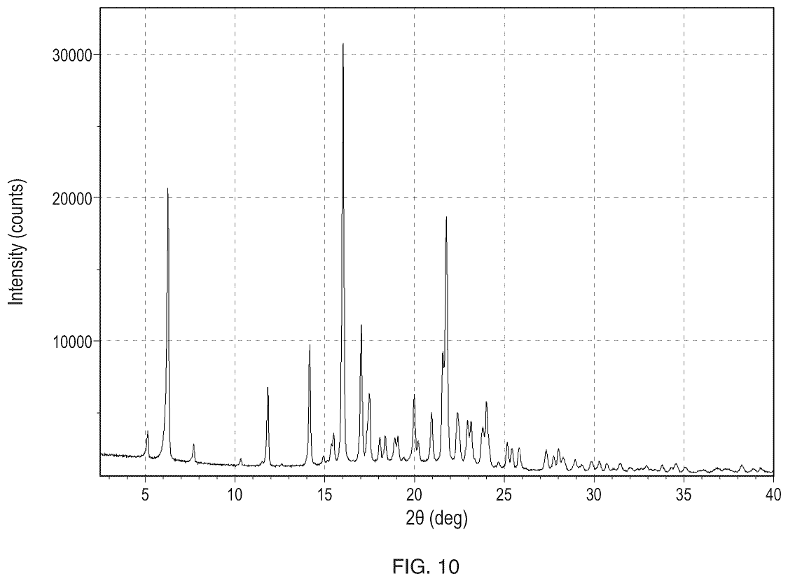

In certain embodiments, the crystalline p-dioxane solvate is characterized by an XRPD pattern substantially the same as shown in FIG. 10.

In certain embodiments, the crystalline p-dioxane solvate exists in an orthorhombic crystal system and has a P2₁2₁2₁ space group. In certain embodiments, the crystalline p-dioxane is characterized by the crystallographic unit cell parameters as set forth in Table 3.

TABLE 3
Unit Cell Parameters of Crystalline p-Dioxane Solvate

| | | |
|---|---|---|
| Unit cell dimensions | a = 6.316 Å | α = 90° |
| | b = 15.364 Å | β = 90° |
| | c = 34.166 Å | γ = 90° |

| | |
|---|---|
| Volume | 3315.4 Å³ |

In certain embodiments, the crystalline p-dioxane solvate dissolved in dimethylsulfoxide-d₆ containing trimethylsilane is characterized by a ¹H NMR spectrum having one, two, three, four, five, or more peaks at 10.800, 10.202, 10.076, 9.754, 8.989, 8.796, 8.714, 8.622, 8.586, 8.547, 8.496, 8.395, 8.360, 8.325, 8.316, 8.303, 8.288, 8.260, 8.199, 8.147, 8.061, 8.042, 7.871, 7.819, 7.799, 7.773, 7.741, 7.738, 7.735, 7.716, 7.699, 7.697, 7.695, 7.694, 7.685, 7.684, 7.682, 7.680, 7.647, 7.611, 7.584, 7.571, 7.557, 7.541, 7.477, 7.464, 7.451, 7.438, 7.425, 7.369, 7.329, 7.304, 7.271, 7.258, 7.245, 7.234, 7.226, 7.198, 7.185, 7.176, 7.141, 7.135, 7.116, 7.063, 7.050, 7.004, 6.974, 6.961, 6.911, 6.899, 6.863, 6.835, 6.822, 6.789, 6.541, 6.528, 6.508, 6.497, 4.268, 4.250, 4.134, 4.124, 4.116, 4.106, 3.694, 3.688, 3.686, 3.682, 3.678, 3.673, 3.652, 3.648, 3.632, 3.604, 3.593, 3.569, 3.554, 3.543, 3.511, 3.455, 3.451, 3.447, 3.445, 3.440, 3.427, 3.401, 3.379, 3.371, 3.360, 3.350, 3.321, 3.308, 3.297, 3.247, 3.226, 3.214, 3.209, 3.196, 3.182, 3.136, 2.743, 2.728, 2.618, 2.615, 2.612, 2.524, 2.521, 2.509, 2.506, 2.503, 2.500, 2.497, 2.390, 2.387, 2.384, 2.079, 2.074, 2.066, 2.061, 1.891, 1.874, 1.850, 1.846, 1.836, 1.828, 1.822, 1.812, 1.803, 1.799, 1.793, 1.788, 1.785, 1.779, 1.773, 1.768, 1.756, 1.751, 1.730, 1.716, 1.712, 1.698, 1.684, 1.668, 1.656, 1.638, 1.634, 1.603, 1.598, 1.586, 1.581, 1.486, 1.480, 1.474, 1.460, 1.454, 1.445, 1.439, 1.425, 1.418, 1.412, 1.244, 1.233, 0.884, 0.878, 0.858, 0.846, 0.831, 0.820, and 0.791 ppm.

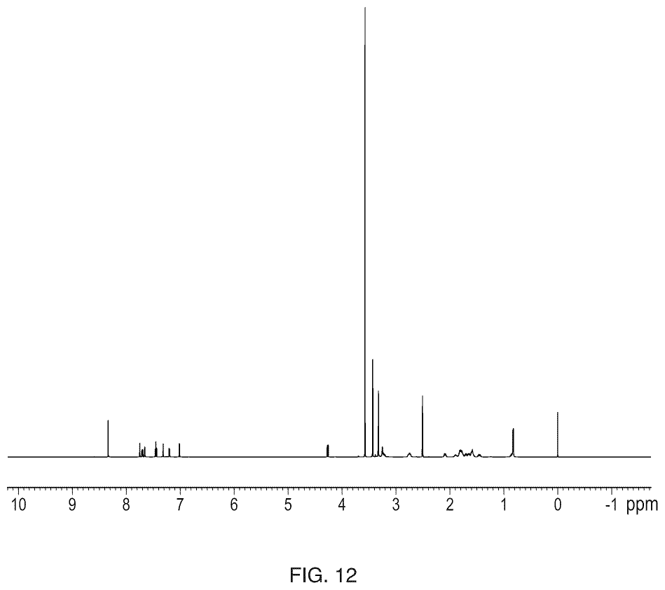

In certain embodiments, the crystalline p-dioxane solvate is characterized by a ¹H NMR spectrum substantially the same as shown in FIG. 12.

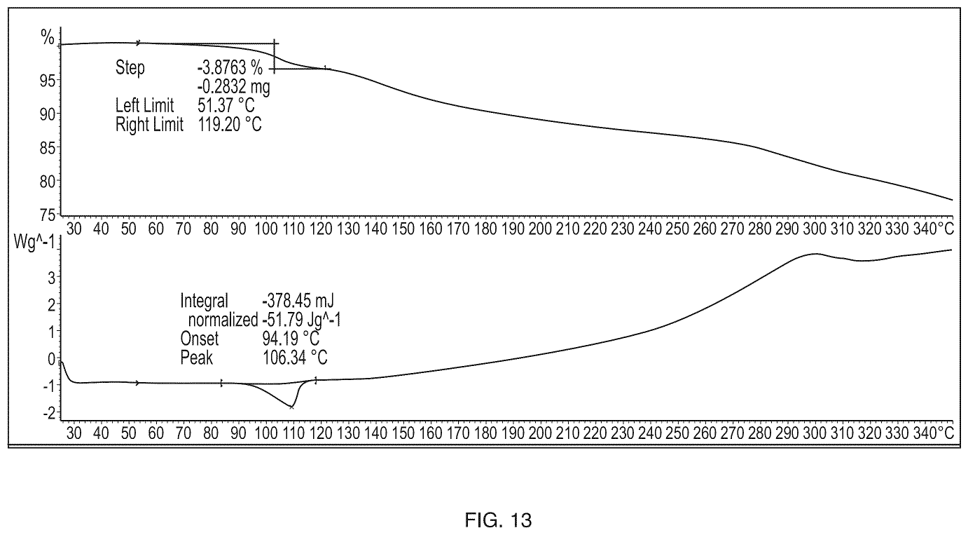

The crystalline p-dioxane solvate of the compound of formula (I) may also be characterized using a thermoanalytical technique, such as DSC. Accordingly, in certain embodiments, the crystalline p-dioxane solvate is characterized by an endotherm with a peak maximum at about 106° C., as determined by DSC. In certain embodiments, the crystalline p-dioxane solvate is characterized by an endotherm with a peak onset of about 94° C., as determined by DSC. In certain embodiments, the crystalline p-dioxane solvate has a DSC thermogram substantially the same as shown in FIG. 13.

The crystalline p-dioxane solvate may also be characterized according to its mass gain/mass loss as a function of temperature. Accordingly, in certain embodiments, the crystalline p-dioxane solvate exhibits a reduction in mass, as determined by TGA, of less than or equal to about 3.9% wt. upon heating to about 119° C. In certain embodiments, the crystalline p-dioxane solvate has a TGA thermogram substantially the same as shown in FIG. 13.

In certain embodiments, the crystalline p-dioxane solvate is a mono-p-dioxane solvate.

### (iv) Crystalline Tetrahydrofuran (THF) Solvate

In various embodiments, provided herein is a crystalline THF solvate of a compound of formula (I):

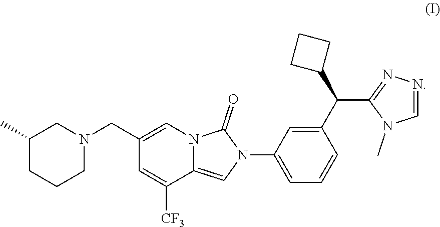 [SMILES: C[C@H]1CCCN(Cc2cc(C(F)(F)F)c3cn(-c4cccc([C@H](c5nncn5C)C5CCC5)c4)c(=O)n3c2)C1]

In certain embodiments, the crystalline THF solvate is characterized by an XRPD pattern comprising one or more peaks selected from 6.3°±0.2°, 16.1°±0.2°, 17.3°±0.2°, and 22.9°±0.2°2-theta.

In certain embodiments, the crystalline THF solvate is characterized by an XRPD pattern comprising peaks at 6.3°±0.2°, 16.1°±0.2°, 17.3°±0.2°, and 22.9°±0.2°2-theta.

In certain embodiments, the XRPD pattern further comprises one or more peaks selected from 17.1°±0.2°, 17.9°±0.20, and 22.4°±0.20 2-theta.

In certain embodiments, the XRPD pattern further comprises peaks at 17.10°±0.2° 17.9°±0.2°, and 22.4°±0.2°2-theta.

In certain embodiments, the crystalline THF solvate is characterized by an XRPD pattern comprising one or more peaks selected from 6.3°±0.2°, 16.1°±0.2°, 17.1°±0.2°, 17.3°±0.2°, 17.9°±0.2°, 22.4°±0.2°, and 22.9°±0.2°2-theta.

In certain embodiments, the crystalline THF solvate is characterized by an XRPD pattern comprising peaks at 6.3°±0.2°, 16.1°±0.2°, 17.1°±0.2°, 17.3°±0.2°, 17.9°±0.2°, 22.4°±0.2°, and 22.9°±0.2°2-theta.

In certain embodiments, the XRPD pattern further comprises one or more peaks selected from 7.7°±0.2°, 11.4°±0.2°, 11.7°±0.2°, 11.9°±0.2°, 12.5°±0.2°, 14.3°±0.2°, 15.2°±0.20, 15.5°±0.2°, 18.3°±0.2°, 18.9°±0.2°, 19.8°±0.2°, 20.1°±0.2°, 21.0°±0.2°, 21.7°±0.20, 21.9°±0.2°, 23.5°±0.2°, 24.1°±0.2°, 24.6°±0.2°, 25.3°±0.2°, 25.9°±0.2°, 27.1°±0.20, 27.3°±0.2°, 28.4°±0.2°, 28.9°±0.2°, 29.5°±0.2°, 30.0°±0.20, and 30.8°±0.20 2-theta.

In certain embodiments, the XRPD pattern further comprises peaks at 7.7°±0.2°, 11.4°±0.2°, 11.7°±0.2°, 11.9°±0.2°, 12.5°±0.2°, 14.3°±0.2°, 15.2°±0.2°, 15.5°±0.2°, 18.30±0.20, 18.9°±0.2°, 19.8°±0.2°, 20.1°±0.2°, 21.0°±0.2°, 21.7°±0.2°, 21.9°±0.2°, 23.50±0.20, 24.1°±0.2°, 24.6°±0.2°, 25.3°±0.2°, 25.9°±0.2°, 27.1°±0.2°, 27.3°±0.2°, 28.40±0.2°, 28.9°±0.2°, 29.5°±0.2°, 30.0°±0.2°, and 30.8°±0.2°2-theta.

In certain embodiments, the crystalline THF solvate is characterized by an XRPD pattern comprising one or more peaks selected from 6.3°±0.2°, 7.7°±0.2°, 11.4°±0.2°, 11.7°±0.2°, 11.9°±0.2°, 12.5°±0.2°, 14.3°±0.2°, 15.2°±0.2°, 15.5°±0.2°, 16.1°±0.2°, 17.10±0.20, 17.3°±0.2°, 17.9°±0.2°, 18.3°±0.2°, 18.9°±0.2°, 19.8°±0.2°, 20.1°±0.2°, 21.00±0.20, 21.7°±0.2°, 21.9°±0.2°, 22.4°±0.2°, 22.9°±0.2°, 23.5°±0.2°, 24.1°±0.2°, 24.60±0.20, 25.3°±0.2°, 25.9°±0.2°, 27.1°±0.2°, 27.3°±0.2°, 28.4°±0.2°, 28.9°±0.2°, 29.50±0.2°, 30.0°±0.2°, and 30.8°±0.2°2-theta.

In certain embodiments, the crystalline THF solvate is characterized by an XRPD pattern comprising peaks at 6.3°±0.2°, 7.7°±0.2°, 11.4°±0.2°, 11.7°±0.2°, 11.9°±0.2° 12.5°±0.2°, 14.3°±0.2°, 15.2°±0.2°, 15.5°±0.2°, 16.1°±0.2°, 17.1°±0.2°, 17.3°±0.20, 17.9°±0.2°, 18.3°±0.2°, 18.9°±0.2°, 19.8°±0.2°, 20.1°±0.2°, 21.0°±0.2°, 21.7°±0.20, 21.9°±0.2°, 22.4°±0.2°, 22.9°±0.2°, 23.5°±0.2°, 24.1°±0.2°, 24.6°±0.2°, 25.3°±0.20, 25.9°±0.2°, 27.1°±0.2°, 27.3°±0.2°, 28.4°±0.2°, 28.9°±0.2°, 29.5°±0.2°, 30.0°±0.20, and 30.8°±0.2°2-theta.

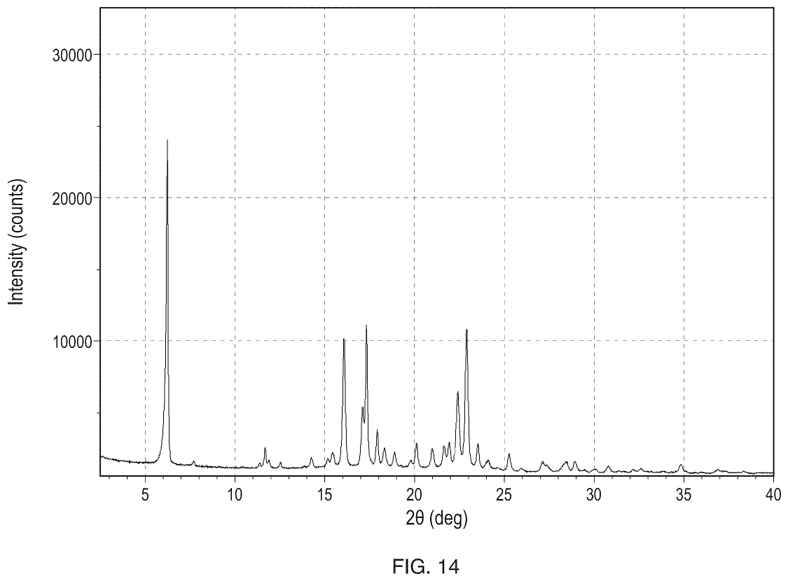

In certain embodiments, the crystalline THF solvate is characterized by an XRPD pattern substantially the same as shown in FIG. 14.

In certain embodiments, the crystalline THF solvate exists in an orthorhombic crystal system and has a P2₁2₁2₁ space group. In certain embodiments, the crystalline THF solvate is characterized by the crystallographic unit cell parameters as set forth in Table 4.

TABLE 4
Unit Cell Parameters of Crystalline THF Solvate

| | | |
|---|---|---|
| Unit cell dimensions | a = 6.289 Å | α = 90° |
| | b = 15.515 Å | β = 90° |
| | c = 33.823 Å | γ = 90° |

| | |
|---|---|
| Volume | 3300.2 Å³ |

In certain embodiments, the crystalline THF solvate dissolved in dimethylsulfoxide-d₆ containing trimethylsilane is characterized by a ¹H NMR spectrum having one, two, three, four, five, or more peaks at 10.797, 10.076, 8.712, 8.568, 8.322, 8.314, 8.301, 8.285, 7.738, 7.735, 7.732, 7.696, 7.695, 7.693, 7.691, 7.683, 7.682, 7.679, 7.678, 7.644, 7.451, 7.438, 7.425, 7.304, 7.197, 7.184, 7.004, 4.266, 4.249, 4.123, 4.105, 3.719, 3.692, 3.674, 3.633, 3.628, 3.622, 3.618, 3.615, 3.612, 3.611, 3.608, 3.605, 3.601, 3.597, 3.594, 3.592, 3.590, 3.588, 3.585, 3.580, 3.574, 3.570, 3.551, 3.541, 3.477, 3.426, 3.399, 3.377, 3.369, 3.358, 3.348, 3.317, 3.293, 3.243, 3.223, 3.212, 3.207, 3.194, 3.180, 3.134, 2.741, 2.616, 2.613, 2.610, 2.607, 2.538, 2.522, 2.519, 2.516, 2.507, 2.504, 2.501, 2.498, 2.495, 2.388, 2.385, 2.382, 2.108, 2.096, 2.090, 2.084, 2.078, 2.072, 2.065, 1.888, 1.870, 1.845, 1.836, 1.827, 1.821, 1.808, 1.803, 1.799, 1.795, 1.793, 1.788, 1.783, 1.779, 1.771, 1.766, 1.760, 1.757, 1.754, 1.749, 1.737, 1.729, 1.714, 1.710, 1.697, 1.683, 1.660, 1.639, 1.582, 1.521, 1.480, 1.460, 1.441, 1.398, 1.355, 1.235, 0.862, 0.833, 0.822, 0.792, and 0.781 ppm.

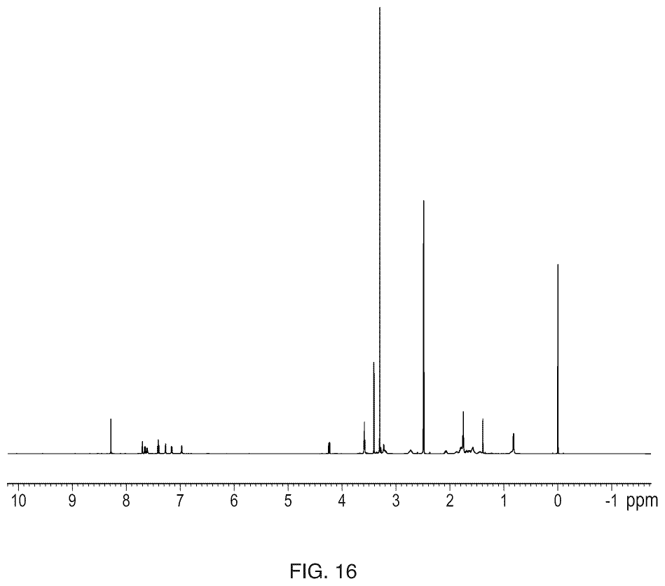

In certain embodiments, the crystalline THF solvate is characterized by a ¹H NMR spectrum substantially the same as shown in FIG. 16.

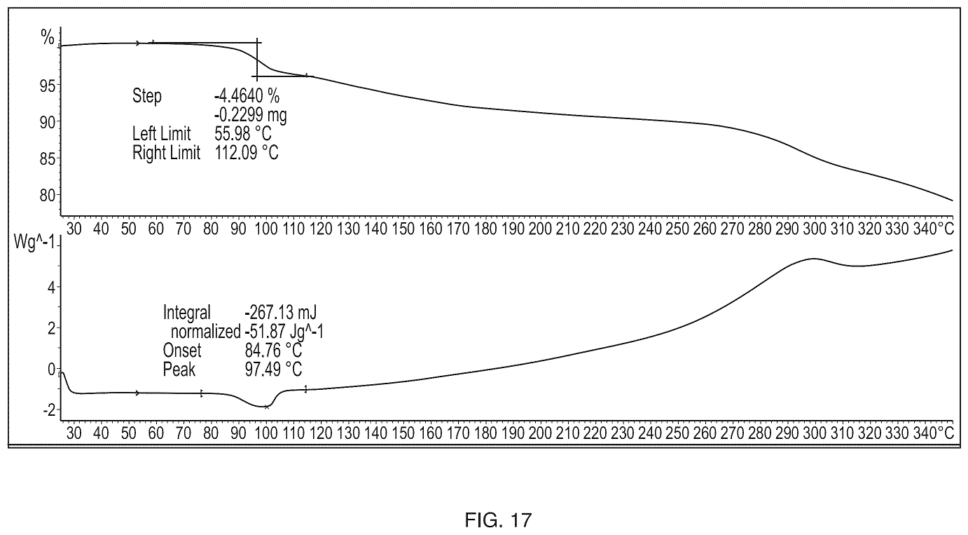

The crystalline THF solvate of the compound of formula (I) may also be characterized using a thermoanalytical technique, such as DSC. Accordingly, in certain embodiments, the crystalline THF solvate is characterized by an endotherm with a peak maximum at about 97° C., as determined by DSC. In certain embodiments, the crystalline THF solvate is characterized by an endotherm with a peak onset of about 85° C., as determined by DSC. In certain embodiments, the crystalline THF solvate has a DSC thermogram substantially the same as shown in FIG. 17.

The crystalline THF solvate may also be characterized according to its mass gain/mass loss as a function of temperature. Accordingly, in certain embodiments, the crystalline THF solvate exhibits a reduction in mass, as determined by TGA, of less than or equal to about 4.5% wt. upon heating to about 112° C. In certain embodiments, the crystalline THF solvate has a TGA thermogram substantially the same as shown in FIG. 17.

In certain embodiments, the crystalline THF solvate is a mono-THF solvate.

### 2. Crystalline Citrate Salt Forms

In one aspect, provided herein is a crystalline citrate salt of a compound of formula (I):

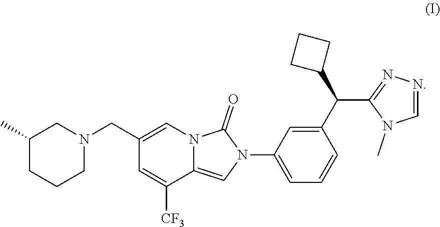 [SMILES: C[C@H]1CCCN(Cc2cc(C(F)(F)F)c3cn(-c4cccc([C@H](c5nncn5C)C5CCC5)c4)c(=O)n3c2)C1]

In certain embodiments, the crystalline citrate salt is an anhydrous crystalline citrate salt. In certain embodiments, the crystalline citrate salt is a solvated crystalline citrate salt (e.g., an acetone solvate, an acetonitrile solvate). In certain embodiments, a crystalline citrate salt described herein is a mono-citrate salt.

### (i) Acetone Solvated Crystalline Citrate Salt

In certain embodiments, the crystalline citrate salt is an acetone solvated crystalline citrate salt.

In certain embodiments, the acetone solvated crystalline citrate salt is characterized by an XRPD pattern comprising one or more peaks selected from 5.°±0.2, 6.6±0.2, 17.6±0.2°, and 18.2±0.2°2-theta.

In certain embodiments, the acetone solvated crystalline citrate salt is characterized by an XRPD pattern comprising peaks at 5.°±0.2, 6.6±0.2°, 17.6±0.2, and 18.2±0.2°2-theta.

In certain embodiments, the XRPD pattern further comprises one or more peaks selected from 15.°±0.2°, 15.3±0.2°, 16.9±0.2°, 19.7±0.2°, 20.1±0.2°, 22.6±0.2°, 22.8±0.2°, and 24.8±0.2°2-theta.

In certain embodiments, the XRPD pattern further comprises peaks at 15.°±0.2°, 15.3±0.2°, 16.9±0.2°, 19.7±0.2, 20.1±0.2, 22.6±0.2°, 22.8±0.2°, and 24.8±0.2°2-theta.

In certain embodiments, the acetone solvated crystalline citrate salt is characterized by an XRPD pattern comprising one or more peaks selected from 5.°±0.2, 6.6±0.2, 15.°±0.2°, 15.3±0.2°, 16.9±0.2°, 17.6±0.2°, 18.2±0.2°, 19.7±0.2°, 20.1±0.2°, 22.6±0.2°, 22.8±0.2°, and 24.8±0.2°2-theta.

In certain embodiments, the acetone solvated crystalline citrate salt is characterized by an XRPD pattern comprising peaks at 5.°±0.2, 6.6±0.2°, 15.°±0.2, 15.3±0.2, 16.9±0.2°, 17.6±0.2°, 18.2±0.2°, 19.7±0.2°, 20.1±0.2°, 22.6±0.2°, 22.8±0.2°, and 24.8±0.2°2-theta.

In certain embodiments, the XRPD pattern further comprises one or more peaks selected from 8.6±0.2°, 10.0 0.2°, 11.°±0.2°, 11.5±0.2°, 13.2±0.2°, 13.3±0.2°, 14.6±0.2°, 15.9±0.20, 16.3±0.2°, 16.5±0.2°, 21.°±0.2°, 21.5±0.2°, 21.8±0.2°, 23.2±0.2°, 23.4±0.2°, 23.9±0.2°, 24.6±0.2°, 25.2±0.2, 26.°±0.2°, 26.6±0.2°, 27.3±0.2°, 28.9±0.2°, 29.5±0.2°, 29.8±0.2, and 30.4±0.2°2-theta.

In certain embodiments, the XRPD pattern further comprises peaks at 8.6±0.2°, 10.0±0.2°, 11.°±0.2°, 11.5±0.2°, 13.2±0.2°, 13.3±0.2°, 14.6±0.2°, 15.9±0.2°, 16.3±0.2°, 16.5±0.2°, 21.°±0.2°, 21.5±0.2°, 21.8±0.2°, 23.2±0.2°, 23.4±0.2°, 23.9±0.2°, 24.6±0.2°, 25.2±0.2, 26.°±0.2, 26.6±0.2°, 27.3±0.2°, 28.9±0.2, 29.5±0.2°, 29.8±0.2°, and 30.4±0.2°2-theta.

In certain embodiments, the acetone solvated crystalline citrate salt is characterized by an XRPD pattern comprising one or more peaks selected from 5.°±0.2, 6.6±0.2, 8.6±0.2°, 10.°±0.2°, 11.0 0.2°, 11.5±0.2°, 13.2±0.2°, 13.3±0.2°, 14.6±0.2°, 15.°±0.2°, 15.3±0.2°, 15.9±0.2°, 16.3±0.2°, 16.5±0.2°, 16.9±0.2°, 17.6±0.2°, 18.2±0.2°, 19.7±0.2°, 20.1±0.2°, 21.°±0.2°, 21.5±0.2°, 21.8±0.2°, 22.6±0.2°, 22.8±0.2°, 23.2±0.2°, 23.4±0.2°, 23.9±0.2°, 24.6±0.2°, 24.8±0.2°, 25.2±0.2°, 26.°±0.2°, 26.6±0.2°, 27.3±0.2°, 28.9±0.2°, 29.5±0.2°, 29.8±0.2°, and 30.4±0.2°2-theta.

In certain embodiments, the acetone solvated crystalline citrate salt is characterized by an XRPD pattern comprising peaks at 5.°±0.2°, 6.6±0.2°, 8.6±0.2°, 10.°±0.2°, 11.°±0.2°, 11.5±0.2°, 13.2±0.2°, 13.3±0.2°, 14.6±0.2°, 15.°±0.2°, 15.3±0.2°, 15.9±0.2°, 16.3±0.2°, 16.5±0.2°, 16.9±0.2°, 17.6±0.2°, 18.2±0.2°, 19.7±0.2°, 20.1±0.2°, 21.°±0.2°, 21.5±0.2°, 21.8±0.2°, 22.6±0.2, 22.8±0.2°, 23.2±0.2°, 23.4±0.2°, 23.9±0.2°, 24.6±0.2°, 24.8±0.2°, 25.2±0.2°, 26.°±0.2°, 26.6±0.2°, 27.3±0.2°, 28.9±0.2°, 29.5±0.2°, 29.8±0.2°, and 30.4±0.2°2-theta.

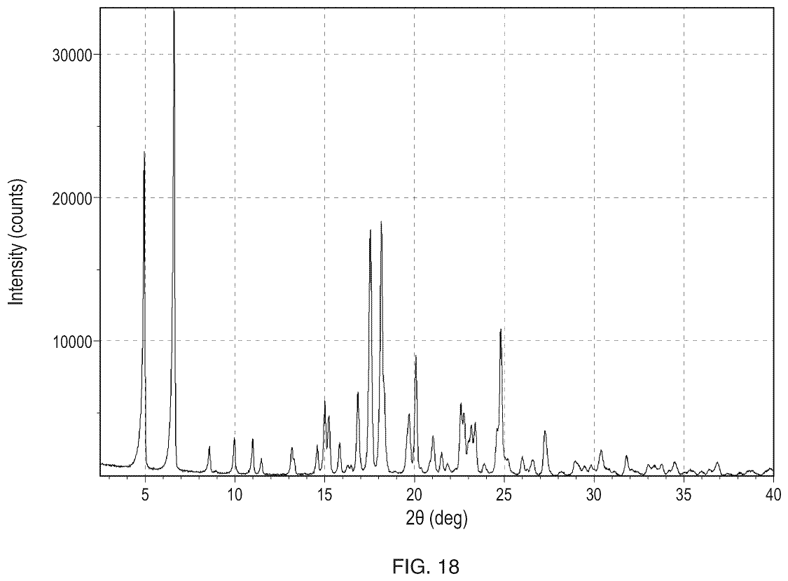

In certain embodiments, the acetone solvated crystalline citrate salt is characterized by an XRPD pattern substantially the same as shown in FIG. 18.

In certain embodiments, the acetone solvated crystalline citrate salt exists in an orthorhombic crystal system and has a P2₁2₁2₁ space group. In certain embodiments, the acetone solvated crystalline citrate salt is characterized by the crystallographic unit cell parameters as set forth in Table 5.

TABLE 5
Unit Cell Parameters of the Acetone
Solvated Crystalline Citrate Salt

| | | |
|---|---|---|
| Unit cell dimensions | a = 6.150 Å | α = 90° |
| | b = 20.489 Å | β = 90° |
| | c = 34.845 Å | γ = 90° |

| | |
|---|---|
| Volume | 4390.7 Å³ |

In certain embodiments, the acetone solvated crystalline citrate salt dissolved in dimethylsulfoxide-d₆ containing trimethylsilane is characterized by ¹H NMR spectrum having one, two, three, four, five, or more peaks at 11.318, 10.869, 10.666, 10.081, 9.432, 8.823, 8.499, 8.327, 8.316, 8.303, 8.288, 8.231, 8.150, 8.136, 8.089, 7.822, 7.723, 7.720, 7.717, 7.696, 7.694, 7.692, 7.691, 7.682, 7.681, 7.679, 7.677, 7.475, 7.462, 7.448, 7.435, 7.346, 7.314, 7.301, 7.288, 7.223, 7.210, 7.082, 7.028, 7.015, 6.559, 6.147, 4.273, 4.256, 3.634, 3.429, 3.401, 3.324, 3.225, 3.213, 3.208, 2.679, 2.654, 2.593, 2.568, 2.509, 2.506, 2.503, 2.500, 2.497, 2.086, 2.081, 2.072, 2.065, 2.059, 2.005, 1.846, 1.837, 1.829, 1.823, 1.814, 1.811, 1.804, 1.801, 1.797, 1.790, 1.786, 1.780, 1.775, 1.768, 1.756, 1.751, 1.732, 1.713, 1.710, 1.699, 1.686, 1.554, 1.533, 1.235, 0.960, 0.942, 0.922, 0.868, 0.858, 0.837, 0.826, 0.762, and 0.752 ppm.

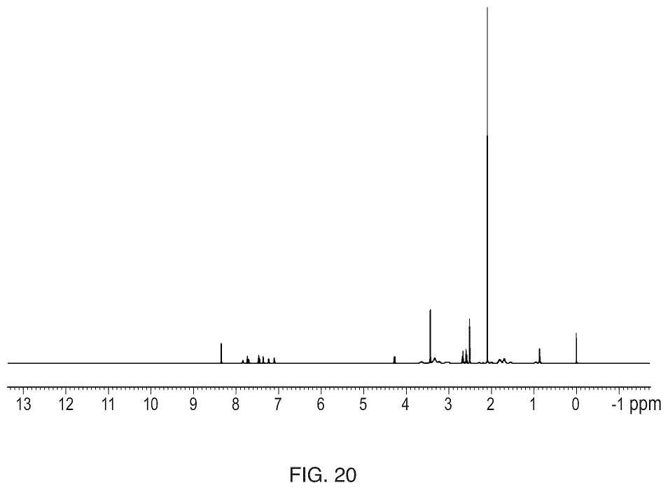

In certain embodiments, the acetone solvated crystalline citrate salt is characterized by a ¹H NMR spectrum substantially the same as shown in FIG. 20.

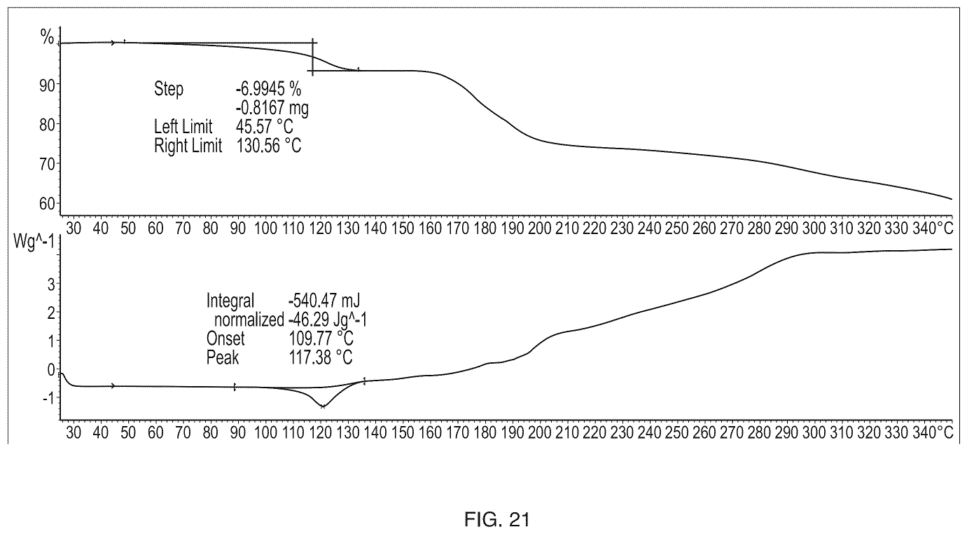

The acetone solvated crystalline citrate salt of the compound of formula (I) may also be characterized using a thermoanalytical technique, such as DSC. Accordingly, in certain embodiments, the acetone solvated crystalline citrate salt is characterized by an endotherm with a peak maximum of about 117° C., as determined by DSC. In certain embodiments, the acetone solvated crystalline citrate salt is characterized by an endotherm with a peak onset of about 110° C., as determined by DSC. In certain embodiments, the acetone solvated crystalline citrate salt has a DSC thermogram substantially the same as shown in FIG. 21.

The acetone solvated crystalline citrate salt may also be characterized according to its mass gain/mass loss as a function of temperature. Accordingly, in certain embodiments, the acetone solvated crystalline citrate salt exhibits a reduction in mass, as determined by TGA, of less than or equal to about 7% wt. upon heating to about 131° C. In certain embodiments, the acetone solvated crystalline citrate salt has a TGA thermogram substantially the same as shown in FIG. 21.

In certain embodiments, the acetone solvated crystalline citrate salt comprises 0.25, 0.5, 0.75, 1, 1.25, 1.5, 1.75, or 2 moles of acetone for every mole of the compound of formula (I).

### (ii) Acetonitrile Solvated Crystalline Citrate Salt

In certain embodiments, the crystalline citrate salt is an acetonitrile solvated crystalline citrate salt.

In certain embodiments, the acetonitrile solvated crystalline citrate salt is characterized by an XRPD pattern comprising one or more peaks selected from 5.1±0.2°, 6.9±0.2, 17.8±0.2°, and 18.7±0.2°2-theta.

In certain embodiments, the acetonitrile solvated crystalline citrate salt is characterized by an XRPD pattern comprising peaks at 5.1±0.2, 6.9±0.2, 17.8±0.2°, and 18.7±0.2°2-theta.

In certain embodiments, the XRPD pattern further comprises one or more peaks selected from 17.2±0.2°, 22.3±0.2°, 23.3±0.2, and 23.6±0.2°2-theta.

In certain embodiments, the XRPD pattern further comprises peaks at 17.2±0.2°, 22.3±0.2°, 23.3±0.2°, and 23.6±0.2°2-theta.

In certain embodiments, the acetonitrile solvated crystalline citrate salt is characterized by an XRPD pattern comprising one or more peaks selected from 5.1±0.2°, 6.9±0.2, 17.2±0.2°, 17.8±0.2°, 18.7±0.2°, 22.3±0.2, 23.3±0.2, and 23.6±0.2°2-theta.

In certain embodiments, the acetonitrile solvated crystalline citrate salt is characterized by an XRPD pattern comprising peaks at 5.1±0.2°, 6.9±0.2°, 17.2±0.2°, 17.8±0.2°, 18.7±0.2°, 22.3±0.2°, 23.3±0.2°, and 23.6±0.2°2-theta.

In certain embodiments, the XRPD pattern further comprises one or more peaks selected from 5.4±0.2°, 8.6±0.2°, 9.2±0.2°, 10.1±0.2°, 10.8±0.2°, 11.7±0.2°, 13.2±0.2°, 13.9±0.2°, 14.°±0.2°, 14.8±0.2°, 15.2±0.2°, 15.4±0.2°, 16.1±0.2°, 16.9±0.2°, 17.4±0.2°, 18.0±0.2°, 19.1±0.2°, 19.5±0.2°, 19.7±0.2°, 19.9±0.2°, 20.2±0.2°, 20.4±0.2°, 20.9±0.2°, 21.1±0.2°, 21.8±0.2°, 21.9±0.2°, 22.6±0.2°, 22.8±0.2°, 23.8±0.2°, 24.1±0.2°, 24.4±0.2°, 25.2±0.2°, 25.5±0.2°, 26.°±0.2°, 26.5±0.2°, 26.7±0.2°, 27.4±0.2°, 27.9±0.2°, 28.4±0.2°2-theta.

In certain embodiments, the XRPD pattern further comprises peaks at 5.4±0.2, 8.6±0.2°, 9.2±0.2°, 10.1±0.2°, 10.8±0.2°, 11.7±0.2°, 13.2±0.2°, 13.9±0.2°, 14.°±0.2°, 14.8±0.2°, 15.2±0.2°, 15.4±0.2°, 16.1±0.2°, 16.9±0.2°, 17.4±0.2°, 18.°±0.2°, 19.1±0.2°, 19.5±0.2°, 19.7±0.2°, 19.9±0.2°, 20.2±0.2°, 20.4±0.2°, 20.9±0.2°, 21.1±0.2°, 21.8±0.2°, 21.9±0.2°, 22.6±0.2°, 22.8±0.2°, 23.8±0.2°, 24.1±0.2°, 24.4±0.2°, 25.2±0.2°, 25.5±0.2°, 26.°±0.2°, 26.5±0.2°, 26.7±0.2, 27.4±0.2, 27.9±0.2°, 28.4±0.2°2-theta.

In certain embodiments, the acetonitrile solvated crystalline citrate salt is characterized by an XRPD pattern comprising one or more peaks selected from 5.1±0.2°, 5.4±0.2, 6.9±0.20, 8.6±0.2°, 9.2±0.2°, 10.1±0.2°, 10.8±0.2°, 11.7±0.2°, 13.2±0.2°, 13.9±0.2°, 14.0±0.20, 14.8±0.2°, 15.2±0.2°, 15.4±0.2°, 16.1±0.2°, 16.9±0.2°, 17.2±0.2°, 17.4±0.2°, 17.8±0.2°, 18.°±0.2°, 18.7±0.2°, 19.1±0.2°, 19.5±0.2°, 19.7±0.2°, 19.9±0.2°, 20.2±0.2°, 20.4±0.2°, 20.9±0.2°, 21.1±0.2°, 21.8±0.2°, 21.9±0.2°, 22.3±0.2°, 22.6±0.2°, 22.8±0.20, 23.3±0.2°, 23.6±0.2°, 23.8±0.2°, 24.1±0.2°, 24.4±0.2°, 25.2±0.2°, 25.5±0.2°, 26.0±0.2°, 26.5±0.2°, 26.7±0.2, 27.4±0.2, 27.9±0.2°, 28.4±0.2°2-theta.

In certain embodiments, the acetonitrile solvated crystalline citrate salt is characterized by an XRPD pattern comprising peaks at 5.1±0.2°, 5.4±0.2°, 6.9±0.2°, 8.6±0.2°, 9.2±0.2°, 10.1±0.2°, 10.8±0.2°, 11.7±0.2°, 13.2±0.2°, 13.9±0.2°, 14.0 0.2°, 14.8±0.2°, 15.2±0.2°, 15.4±0.2°, 16.1±0.2°, 16.9±0.2°, 17.2±0.2°, 17.4±0.2°, 17.8±0.2°, 18.°±0.2°, 18.7±0.2°, 19.1±0.2°, 19.5±0.2°, 19.7±0.2°, 19.9±0.2°, 20.2±0.2°, 20.4±0.2°, 20.9±0.2°, 21.1±0.2°, 21.8±0.2°, 21.9±0.2°, 22.3±0.2°, 22.6±0.2°, 22.8±0.2°, 23.3±0.2°, 23.6±0.2°, 23.8±0.2°, 24.1±0.2°, 24.4±0.2°, 25.2±0.2°, 25.5±0.2°, 26.°±0.2°, 26.5±0.2°, 26.7±0.2°, 27.4±0.2°, 27.9±0.2, 28.4±0.2°2-theta.

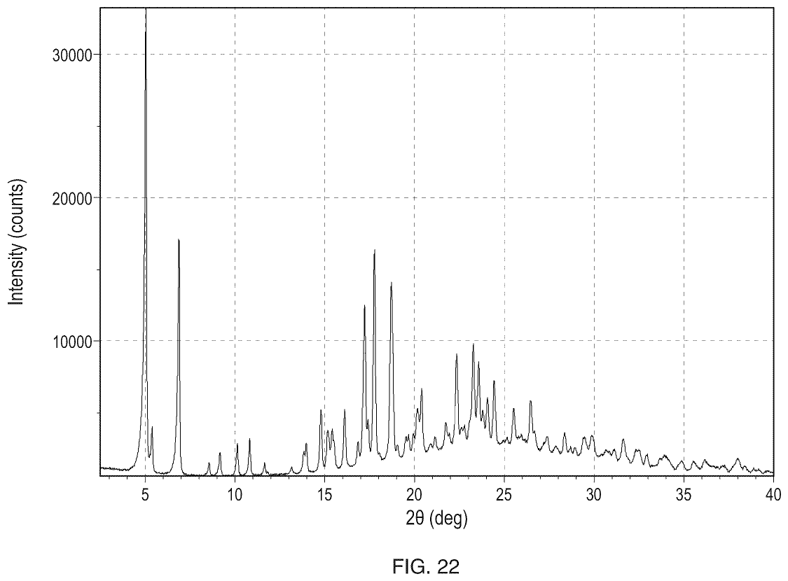

In certain embodiments, the acetonitrile solvated crystalline citrate salt is characterized by an XRPD pattern substantially the same as shown in FIG. 22.

In certain embodiments, the acetonitrile solvated crystalline citrate salt exists in an orthorhombic crystal system and has a P2₁2₁2₁ space group. In certain embodiments, the acetonitrile solvated crystalline citrate salt is characterized by the crystallographic unit cell parameters as set forth in Table 6.

TABLE 6
Unit Cell Parameters of the Acetonitrile
Solvated Crystalline Citrate Salt

| | | |
|---|---|---|
| Unit cell dimensions | a = 6.081 Å | α = 90° |
| | b = 20.605 Å | β = 90° |
| | c = 32.628 Å | γ = 90° |

| | |
|---|---|
| Volume | 4088.3 Å³ |

In certain embodiments, the acetonitrile solvated crystalline citrate salt comprises 0.25, 0.5, 0.75, 1, 1.25, 1.5, 1.75, or 2 moles of acetonitrile for every mole of the compound of formula (I).

(iii) Anhydrous Crystalline Citrate Salt

In certain embodiments, the crystalline citrate salt is an anhydrous crystalline citrate salt.

In certain embodiments, the crystalline citrate salt is characterized by an XRPD pattern comprising one or more peaks selected from 5.3±0.2°, 6.4±0.2, 17.6±0.2°, and 23.°±0.2°2-theta.

In certain embodiments, the crystalline citrate salt is characterized by an XRPD pattern comprising peaks at 5.3±0.2, 6.4±0.2, 17.6±0.2, and 23.°±0.2°2-theta.

In certain embodiments, the XRPD pattern further comprises one or more peaks selected from 8.5±0.2°, 10.6±0.2°, 16.°±0.2°, 17.°±0.2°, 17.3±0.2°, 18.2±0.2°, 21.4±0.2°, and 22.4±0.2°2-theta.

In certain embodiments, the XRPD pattern further comprises peaks at 8.5±0.2°, 10.6±0.2°, 16.°±0.2°, 17.°±0.2°, 17.3±0.2, 18.2±0.2, 21.4±0.2°, and 22.4±0.2°2-theta.

In certain embodiments, the crystalline citrate salt is characterized by an XRPD pattern comprising one or more peaks selected from 5.3±0.2°, 6.4±0.2, 8.5±0.2, 10.6±0.2°, 16.0±0.20, 17.°±0.2°, 17.3±0.2°, 17.6±0.2°, 18.2±0.2, 21.4±0.2°, 22.4±0.20, and 23.°±0.20 2-theta.

In certain embodiments, the crystalline citrate salt is characterized by an XRPD pattern comprising peaks at 5.3±0.2°, 6.4±0.2°, 8.5±0.2°, 10.6±0.2°, 16.0 0.2°, 17.°±0.2°, 17.3±0.2°, 17.6±0.2°, 18.2±0.2°, 21.4±0.2, 22.4±0.2, and 23.°±0.2°2-theta.

In certain embodiments, the XRPD pattern further comprises one or more peaks selected from 7.6±0.2°, 9.1±0.2°, 12.8±0.2°, 14.4±0.2°, 15.4±0.2°, 16.4±0.2°, 16.7±0.2°, 19.0±0.2°, 19.3±0.2°, 19.5±0.2°, 20.1±0.2°, 20.9±0.2°, 24.3±0.2°, 25.7±0.2°, 25.8±0.2°, 26.4±0.2°, 27.4±0.2°, and 28.°±0.2°2-theta.

In certain embodiments, the XRPD pattern further comprises peaks at 7.6±0.2, 9.1±0.20, 12.8±0.2°, 14.4±0.2°, 15.4±0.2°, 16.4±0.2°, 16.7±0.2°, 19.°±0.2°, 19.3±0.2°, 19.5±0.2°, 20.1±0.2°, 20.9±0.2, 24.3±0.2°, 25.7±0.2°, 25.8±0.2°, 26.4±0.2°, 27.4±0.2°, and 28.°±0.2°2-theta.

In certain embodiments, the crystalline citrate salt is characterized by an XRPD pattern comprising one or more peaks selected from 5.3±0.2°, 6.4±0.2°, 7.6±0.2°, 8.5±0.2°, 9.1±0.20, 10.6±0.2°, 12.8±0.2°, 14.4±0.2°, 15.4±0.2°, 16.°±0.2°, 16.4±0.2°, 16.7±0.2°, 17.0±0.2°, 17.3±0.2°, 17.6±0.2°, 18.2±0.2°, 19.°±0.2°, 19.3±0.2°, 19.5±0.2°, 20.1±0.2°, 20.9±0.2°, 21.4±0.2°, 22.4±0.2°, 23.°±0.2°, 24.3±0.2°, 25.7±0.2°, 25.8±0.2°, 26.4±0.2°, 27.4±0.2°, and 28.°±0.2°2-theta.

In certain embodiments, the crystalline citrate salt is characterized by an XRPD pattern comprising peaks at 5.3±0.2°, 6.4±0.2°, 7.6±0.2°, 8.5±0.2°, 9.1±0.2°, 10.6±0.2°, 12.8±0.2°, 14.4±0.2°, 15.4±0.2°, 16.°±0.2°, 16.4±0.2°, 16.7±0.2°, 17.°±0.2°, 17.3±0.2°, 17.6±0.2°, 18.2±0.2°, 19.°±0.2°, 19.3±0.2°, 19.5±0.2°, 20.1±0.2°, 20.9±0.2°, 21.4±0.2°, 22.4±0.2°, 23.0 0.2°, 24.3±0.2°, 25.7±0.2°, 25.8±0.2°, 26.4±0.2°, 27.4±0.2°, and 28.0±0.2°2-theta.

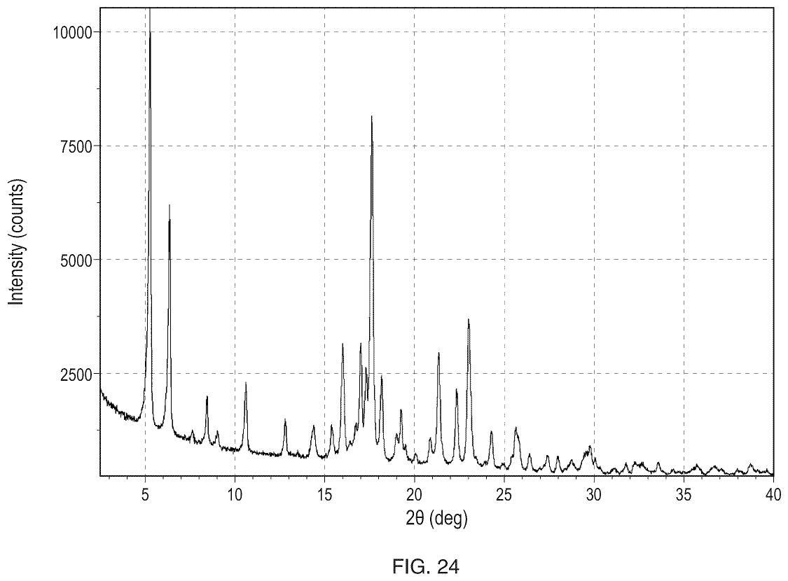

In certain embodiments, the crystalline citrate salt is characterized by an XRPD pattern substantially the same as shown in FIG. 24.

In certain embodiments, the crystalline citrate salt exists in an orthorhombic crystal system and has a P2₁2₁2₁ space group. In certain embodiments, the crystalline citrate salt is characterized by the crystallographic unit cell parameters as set forth in Table 7.

TABLE 7
Unit Cell Parameters of Anhydrous Crystalline Citrate Salt

| | | |
|---|---|---|
| Unit cell dimensions | a = 6.297 Å | α = 90° |
| | b = 20.855 Å | β = 90° |
| | c = 27.665 Å | γ = 90° |

| | |
|---|---|
| Volume | 3633.1 Å³ |

In certain embodiments, the crystalline citrate salt dissolved in dimethylsulfoxide-d₆ containing trimethylsilane is characterized by ¹H NMR spectrum having one, two, three, four, five, or more peaks at 11.967, 8.325, 7.820, 7.718, 7.715, 7.694, 7.693, 7.691, 7.689, 7.681, 7.679, 7.677, 7.676, 7.461, 7.448, 7.434, 7.345, 7.222, 7.209, 7.082, 4.272, 4.254, 3.630, 3.543, 3.428, 3.399, 3.348, 3.317, 3.294, 3.249, 3.237, 3.224, 3.211, 3.194, 3.180, 3.016, 2.683, 2.658, 2.616, 2.613, 2.610, 2.596, 2.570, 2.537, 2.522, 2.519, 2.516, 2.507, 2.504, 2.501, 2.498, 2.495, 2.388, 2.385, 2.382, 2.274, 2.107, 2.094, 2.089, 2.083, 2.077, 2.071, 2.064, 2.010, 1.837, 1.828, 1.822, 1.814, 1.810, 1.803, 1.795, 1.789, 1.785, 1.780, 1.775, 1.767, 1.756, 1.750, 1.731, 1.712, 1.698, 1.685, 1.549, 1.235, 0.960, 0.943, 0.868, and 0.858 ppm.

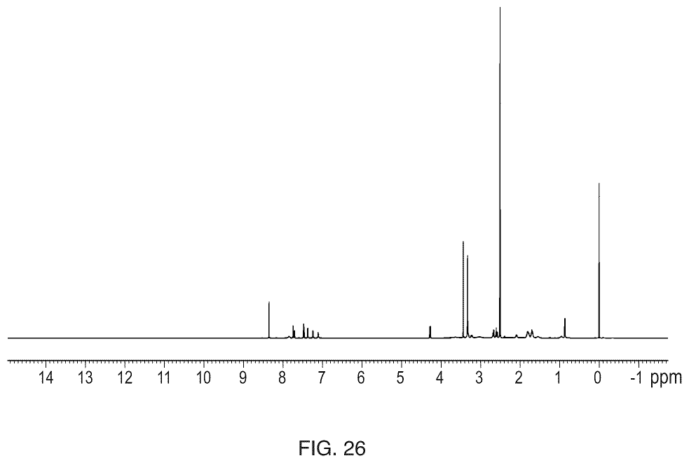

In certain embodiments, the crystalline citrate salt is characterized by a ¹H NMR spectrum substantially the same as shown in FIG. 26.

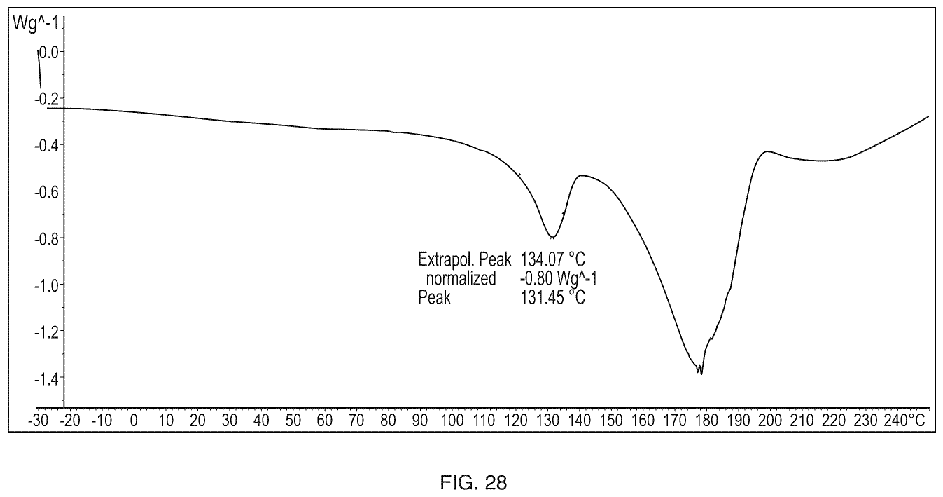

The crystalline citrate salt of the compound of formula (I) may also be characterized using a thermoanalytical technique, such as DSC. Accordingly, in certain embodiments, the crystalline citrate salt is characterized by an endotherm with a peak maximum of about 131° C., as determined by DSC. In certain embodiments, the crystalline citrate salt is characterized by a melting point onset of about 131° C., as determined by DSC. In certain embodiments, the crystalline citrate salt has a DSC thermogram substantially the same as shown in FIG. 28.

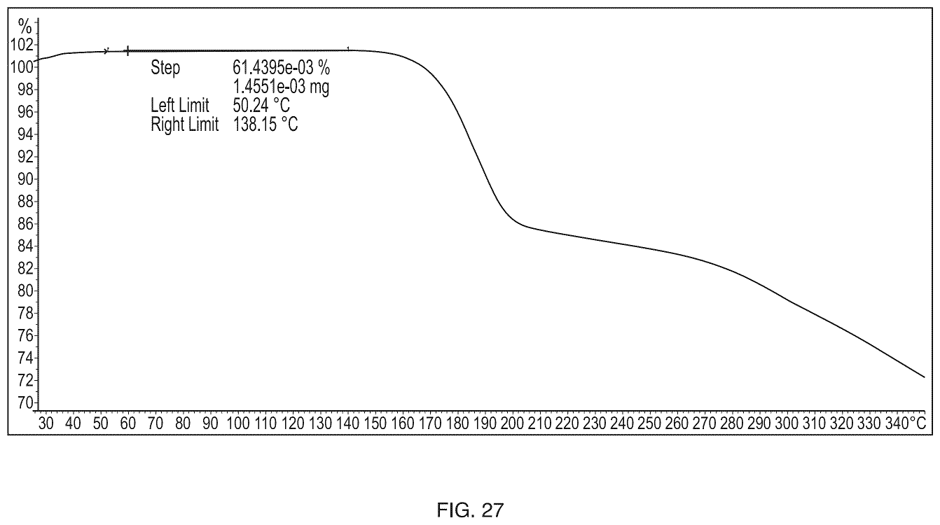

The crystalline citrate salt may also be characterized according to its mass gain/mass loss as a function of temperature. Accordingly, in certain embodiments, the crystalline citrate salt exhibits a reduction in mass, as determined by TGA, of less than or equal to about 0.1% wt. upon heating to about 138° C. In certain embodiments, the crystalline citrate salt has a TGA thermogram substantially the same as shown in FIG. 27.

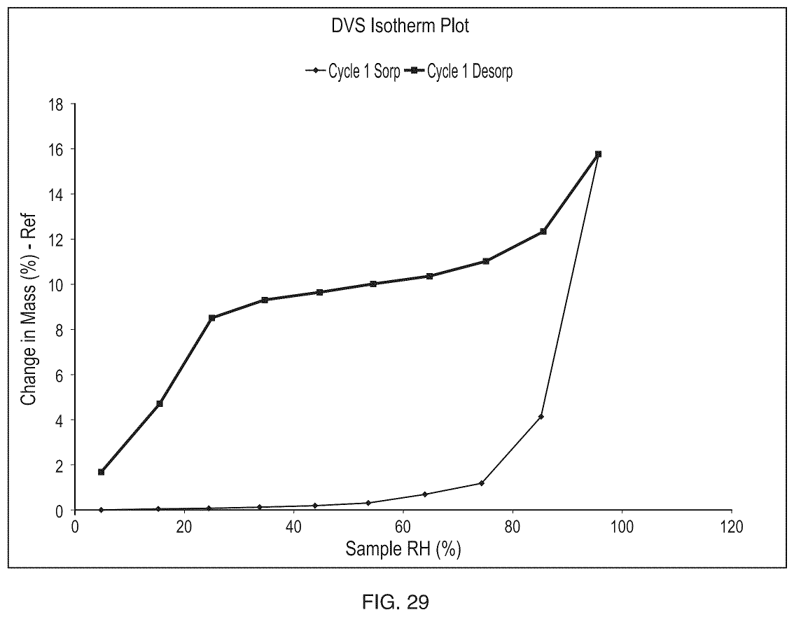

The crystalline citrate salt may also be characterized according to its water sorption properties. Accordingly, in certain embodiments, the crystalline citrate salt exhibits a mass increase, as determined by DVS, of less than or equal to about 1.2% wt. at a relative humidity of 74% and a temperature of 25° C. In certain embodiments, the crystalline citrate salt exhibits a mass increase, as determined by DVS, of less than or equal to about 15.8% wt. at a relative humidity of 96% and a temperature of 25° C. In certain embodiments, the crystalline citrate salt has a water sorption isotherm, when measured at 25° C., substantially the same as shown in FIG. 29.

### Pharmaceutical Compositions and Routes of Administration

The crystalline forms of the compound of formula (I) disclosed herein are usually administered in the form of pharmaceutical compositions. In one aspect, provide herein are pharmaceutical compositions that contain, a crystalline form of the compound of formula (I) described herein, and one or more pharmaceutically acceptable excipients and/or carriers, including, but not limited to, inert solid diluents and fillers, diluents, including sterile aqueous solution and various organic solvents, permeation enhancers, solubilizers and adjuvants. The pharmaceutical compositions described herein may be administered alone or in combination with other therapeutic agents. Such pharmaceutical compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions described herein may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the pharmaceutical compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a crystalline form of the compound of formula (I) according to the present disclosure in the required amount in the appropriate solvent with various other ingredients as enumerated above, as required, followed by, for example, filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the crystalline forms of the compound of formula (I) in accordance with the disclosure. Administration may be via capsule or enteric coated tablets, or the like. In making the pharmaceutical compositions that include a crystalline form of the compound of formula (I) described herein, the active ingredient (e.g., the crystalline form of the compound of formula (I)) is usually diluted by an excipient and/or enclosed within such a carrier that can be in the form of a capsule, sachet, paper, or other container. When the excipient serves as a diluent, it can be in the form of a solid, semi-solid, or liquid material (as above), which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The pharmaceutical compositions of the disclosure can be formulated so as to provide quick, sustained or delayed release of the active ingredient (e.g., the compound of formula (I)) after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compound of formula (I) in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). Compounds, such as the compound of formula (I), are generally administered in a pharmaceutically effective amount. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient (e.g., a crystalline form of the compound of formula (I) described herein) is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills, and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprises a disclosed crystalline form of the compound of formula (I) and a pharmaceutically acceptable carrier.

### Methods of Treatment

In various embodiments, the present disclosure provides a method for treating or lessening the severity of a disease or condition associated with cell proliferation (e.g., a cancer) in a patient comprising the step of administering to said subject a crystalline form of the compound of formula (I) according to the present disclosure.

In various embodiments, provided herein is a method for treating or lessening the severity of a disease or condition associated with cell proliferation (e.g., a cancer) in a patient comprising the step of administering to said subject an effective amount of a crystalline form of the compound of formula (I) according to the present disclosure.

In various embodiments, provided herein is a method for treating or lessening the severity of a disease or condition associated with cell proliferation (e.g., a cancer) in a patient comprising the step of administering to said subject an effective amount of an anhydrous crystalline form of the compound of formula (I) described herein.

In various embodiments, provided herein is a method for treating or lessening the severity of a disease or condition associated with cell proliferation (e.g., a cancer) in a patient comprising the step of administering to said subject an effective amount of a crystalline acetone solvate of the compound of formula (I) described herein.

In various embodiments, provided herein is a method for treating or lessening the severity of a disease or condition associated with cell proliferation (e.g., a cancer) in a patient comprising the step of administering to said subject an effective amount of a crystalline p-dioxane solvate of the compound of formula (I) described herein.

In various embodiments, provided herein is a method for treating or lessening the severity of a disease or condition associated with cell proliferation (e.g., a cancer) in a patient comprising the step of administering to said subject an effective amount of a crystalline tetrahydrofuran solvate of the compound of formula (I) described herein.

In various embodiments, provided herein is a method for treating or lessening the severity of a disease or condition associated with cell proliferation (e.g., a cancer) in a patient comprising the step of administering to said subject an effective amount of an acetone solvated crystalline citrate salt of the compound of formula (I) described herein.

In various embodiments, provided herein is a method for treating or lessening the severity of a disease or condition associated with cell proliferation (e.g., a cancer) in a patient comprising the step of administering to said subject an effective amount of an acetonitrile solvated crystalline citrate salt of the compound of formula (I) described herein.

In various embodiments, provided herein is a method for treating or lessening the severity of a disease or condition associated with cell proliferation (e.g., a cancer) in a patient comprising the step of administering to said subject an effective amount of an anhydrous crystalline citrate salt of the compound of formula (I) described herein.

The term “disease or condition associated with cell proliferation” as used herein means any disease or other deleterious condition in which cell proliferation is known to play a role. Accordingly, certain embodiments of the present disclosure relate to treating or lessening the severity of one or more diseases in which cell proliferation is known to play a role. In certain embodiments, a disease or condition associated with cell proliferation is hyperplasia or a cancer. In certain embodiments, a disease or condition associated with cell proliferation is a cancer.

In certain embodiments, administration of a crystalline form of the present disclosure (e.g., a crystalline form of the compound of formula (I) described herein) results in arrest of mitosis. In certain embodiments, mitotic arrest is defined as a 10-100% reduction in mitosis. In certain embodiments, mitotic arrest is defined as a 20-100% reduction in mitosis. In certain embodiments, mitotic arrest is defined as a 30-100% reduction in mitosis. In certain embodiments, mitotic arrest is defined as a 40-100% reduction in mitosis. In certain embodiments, mitotic arrest is defined as a 50-100% reduction in mitosis. In certain embodiments, mitotic arrest is defined as a 60-100% reduction in mitosis. In certain embodiments, mitotic arrest is defined as a 70-100% reduction in mitosis. In certain embodiments, mitotic arrest is defined as an 80-100% reduction in mitosis. In certain embodiments, mitotic arrest is defined as a 90-100% reduction in mitosis. In certain embodiments, mitotic arrest is defined as a 100% reduction in mitosis.

In certain embodiments, the crystalline forms and pharmaceutical compositions described herein, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Crystalline forms of the present disclosure are preferably formulated in dosage unit form for ease of administration and uniformity of dosage.

In certain embodiments, the cancer is a hematologic cancer. In certain embodiments, the hematologic cancer is selected from a group consisting of a lymphoma, a leukemia, and a myeloma. In certain embodiments, the hematologic cancer is a lymphoma. In certain embodiments, the hematologic cancer is a leukemia. In certain embodiments, a hematologic cancer is a myeloma.

In certain embodiments, the cancer is a non-hematologic cancer. In certain embodiments, the non-hematologic cancer is a sarcoma or a carcinoma. In certain embodiments, the non-hematologic cancer is a sarcoma. In certain embodiments, the non-hematologic cancer is a carcinoma.

In certain embodiments, a subject has one or more of increased T-cell activation, increased T-cell proliferation, decreased T-cell exhaustion, decreased T-cell anergy, and decreased T-cell tolerance after administration of the crystalline form of the present disclosure. In some embodiments, administration of a crystalline form of the present disclosure to a subject in need there of results in one or more of increased T-cell activation, increased T-cell proliferation, decreased T-cell exhaustion, decreased T-cell anergy, and decreased T-cell tolerance.

In certain embodiments, the subject has increased NK-cell activation. In certain embodiments, increased NK-cell activation comprises increased production of cytokines.

In certain embodiments, pharmaceutically acceptable compositions of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, the crystalline forms of the present disclosure may be administered orally or parenterally at dosage levels of about 0.01 mg/kg to about 50 mg/kg and preferably from about 1 mg/kg to about 25 mg/kg of subject body weight per day, one or more times a day, to obtain desired therapeutic effect.

In certain embodiments, one or more additional therapeutic agents, may also be administered in combination with a crystalline form of the compound of formula (I) disclosed herein. In certain embodiments, a crystalline form of the compound of formula (I) disclosed herein and one or more additional therapeutic agents may be administered as part of a multiple dosage regime. In certain embodiments, a crystalline form of the compound of formula (I) disclosed herein and one or more additional therapeutic agents may be administered may be administered simultaneously, sequentially or within a period of time. In certain embodiments, a crystalline form of the compound of formula (I) disclosed herein and one or more additional therapeutic agents may be administered within five hours of one another. In certain embodiments, a crystalline form of the compound of formula (I) disclosed herein and one or more additional therapeutic agents may be administered within 24 hours of one another. In certain embodiments, a crystalline form of the compound of formula (I) disclosed herein and one or more additional therapeutic agents may be administered within one week of one another.

In certain embodiments, a crystalline form of the compound of formula (I) disclosed herein and one or more additional therapeutic agents may be formulated into a single dosage form (e.g., a fixed-dose combination).

### EXAMPLES

The representative examples that follow are intended to help illustrate the disclosure, and are not intended to, nor should they be construed to, limit the scope of the disclosure.

### Abbreviations

ACN Acetonitrile
DCM Dichloromethane
DIPE Di-isopropyl ether
DSC Differential scanning calorimetry
DVS Dynamic vapor sorption
EtOAc Ethyl acetate
EtOH Ethanol
IPA Isopropanol
IPOAc Isopropyl acetate
MeOH Methanol
MBTE Methyl t-butyl ether
¹H NMR Proton nuclear magnetic resonance
TGA Thermogravimetric analysis
THF Tetrahydrofuran
XRD X-ray diffraction
XRPD X-ray powder diffraction
wt. weight

### Example 1—Analytical Methods

### (i) XRPD

XRPD data provided herein were collected with a PANalytical X'Pert PRO MPD diffractometer using an incident beam of Cu radiation produced by an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the Si 111 peak position. A specimen of the sample was sandwiched between 3 μm thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v.5.5.

### (ii) XRPD Peak Identification

Under most circumstances, peaks within the range of up to about 30° 20 were selected. Rounding algorithms were used to round each peak to the nearest 0.1° 2θ. The location of the peaks along the x-axis (° 20) in both the figures and the tables were determined using proprietary software and rounded to one significant figure after the decimal point. Peak position variabilities are given to within ±0.2°, 20 based upon recommendations outlined in the USP discussion of variability in x-ray powder diffraction. For d-space listings, the wavelength used to calculate d-spacings was 1.5405929 Å, the Cu-Kai wavelength. Variability associated with d-spacing estimates was calculated from the USP recommendation, at each d-spacing, and provided in the respective data tables.

(iii) Indexing XRPD Data

The XRPD patterns of the crystalline forms described in Examples 3-8 were indexed using X'Pert High-Score Plus 2.2a (2.2.1). Successful indexing of a pattern indicates that the sample is composed primarily or exclusively of a single crystalline phase unless otherwise stated. Space groups consistent with the assigned extinction symbol, unit cell parameters, and derived quantities were tabulated.

### (iv) Single Crystal X-ray Diffraction

Standard uncertainty is written in crystallographic parenthesis notation, e.g., 0.123(4) is equivalent to 0.123±0.004.

### (a) Data Collection

A light orange needle of the anhydrous form of the compound of formula (I) (described in Example 2) having approximate dimensions of 0.27×0.04×0.03 mm³, was mounted on a polymer loop in random orientation. Preliminary examination and data collection were performed on a Rigaku SuperNova diffractometer, equipped with a copper anode microfocus sealed X-ray tube (Cu Kα k=1.54184 Å) and a Dectris Pilatus3 R 200K hybrid pixel array detector.

Cell constants and an orientation matrix for data collection were obtained from least-squares refinement using the setting angles of 7350 reflections in the range 4.3060°<0<75.1220°. The space group was determined by the program CRYSALISPRO (CrysAlisPro 1.171.41.93a (Rigaku Oxford Diffraction, 2020)) to be P2₁2₁2₁ (international tables no. 19).

The data were collected to a maximum diffraction angle (20) of 151.71° at room temperature.

### (b) Data Reduction

Frames were integrated with CRYSALISPRO. A total of 13966 reflections were collected, of which 5799 were unique. Lorentz and polarization corrections were applied to the data. The linear absorption coefficient is 0.762 mm⁻¹ for Cu Kα radiation. An empirical absorption correction using CRYSALISPRO was applied. Transmission coefficients ranged from 0.951 to 1.000. Intensities of equivalent reflections were averaged. The agreement factor for the averaging was 2.11% based on intensity.

### (c) Structure Solution and Refinement

The structure was solved by direct methods using SHELXT (Sheldrick, G. M. Acta Cryst. 2015, A71, 3-8). The remaining atoms were located in succeeding difference Fourier syntheses. The structure was refined using SHELXL-2014 (Sheldrick, G. M. Acta Cryst., 2008, A64, 112-122.). Hydrogen atoms were included in the refinement but restrained to ride on the atom to which they are bonded. The structure was refined in full-matrix least-squares by minimizing the function:

$${{\sum w [ {F}_{o} \vert}^{2} - {\vert {F}_{c} \vert}^{2} )}^{2}$$

where the weight, w, is defined as 1/[σ²(F₀ ²)+(0.0834P)²+(0.2745P)], where P=(F₀ ²+2F_{c}²)/3.

Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4.). Of the 5799 reflections used in the refinements, only the reflections with intensities larger than twice their uncertainty [I>2σ(I) ], 5239, were used in calculating the fit residual, R. The final cycle of refinement included 354 variable parameters, 0 restraints, and converged with respective unweighted and weighted agreement factors of:

$$\begin{matrix}R = \sum \vert {F}_{o} - {F}_{c} \vert / \sum {F}_{o} = 0.0456 \\ {R}_{w} = \sqrt{( \sum {w ( {F}_{o}^{2} - {F}_{c}^{2} )}^{2} / \sum {w ( {F}_{o}^{2} )}^{2} )} = 0.1272\end{matrix}$$

The standard deviation of an observation of unit weight (goodness of fit) was 1.05. The highest peak in the final difference Fourier had an electron density of 0.315 e/Å³. The minimum negative peak had a value of −0.255 e/Å³.

### (d) Calculated XRPD Pattern

A calculated XRPD pattern was generated for Cu radiation using MERCURY (Macrae, C. F. Edgington, P. R. McCabe, P. Pidcock, E. Shields, G. P. Taylor, R. Towler M. and van de Streek, J. J. Appl. Cryst., 2006, 39, 453-457.) and the atomic coordinates, space group, and unit cell parameters from the single crystal structure.

### (e) Atomic Displacement Ellipsoid and Packing Diagrams

The atomic displacement ellipsoid diagram was prepared using MERCURY. Atoms are represented by 50% probability anisotropic thermal ellipsoids.

### (v) ¹H NMR

The solution nuclear magnetic resonance spectra were acquired with an Avance 600 MHz NMR Spectrometer. The samples of the crystalline forms described herein were prepared by dissolving approximately 4-7 mg of a sample in dimethylsulfoxide-d₆ containing trimethylsilane.

### (vi) DSC/TGA

DSC/TGA analysis was performed using a Mettler-Toledo TGA/DSC3+ analyzer. Temperature calibration was performed using calcium oxalate, indium, tin, and zinc. The sample was placed in an aluminum pan. The sample was sealed, the lid pierced, then inserted into the TG furnace. The furnace was heated under nitrogen with flow of 50 mg/mL. The usual method includes heating from ambient temperature to 350° C. with heating rate 10° C./min.

(vii) DVS

Vapor sorption data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours.

TABLE 8
Hygroscopicity Classifications (Ph. Eur. 10.0)

| Classification | Weight Increase at 80% RH (25° C.) |
|---|---|
| Non hygroscopic | <0.2% |
| Slightly hygroscopic | ≥0.2% and <2% |
| Hygroscopic | ≥2% and <15% |
| Very hygroscopic | ≥15% |
| Deliquescent | sufficient water is absorbed to form a liquid |

### Example 2—Anhydrous Crystalline Form of the Compound of Formula (I)

### (i) Preparation of the Anhydrous Crystalline Form

The anhydrous crystalline form of the compound of formula (I) was prepared as follows: 55 mg of the compound of formula (I) was dissolved in ethyl acetate (0.3 ml) at 60° C. The solution was filtered through a 0.2 μm nylon filter into a pre-warmed (60° C.) vial. The vial was capped and the sample was placed in the freezer at −15 to −25° C. Solids precipitated from solution and were isolated by syringe filtration.

Single crystals of the anhydrous crystalline form of the compound of formula (I) for single crystal XRD analysis were prepared as follows: 104 mg of the compound of formula (I) was dissolved in acetonitrile (1 ml) at ˜60° C. The solution was then placed at ambient temperature for cooling. Suitable single crystals were then harvested for analysis.

### (ii) Characterization of the Anhydrous Crystalline Form

An XRPD pattern for the anhydrous crystalline form of the compound of formula (I) is provided in FIG. 1. Tabulated characteristics of the XRPD pattern in FIG. 1 are provided in Table 9, which lists diffraction angle 2θ, d-spacing [Å], and relative intensity (expressed as a percentage with respect to the most intense peak).

An ¹H NMR spectrum for the anhydrous crystalline form of the compound of formula (I) dissolved in dimethylsulfoxide-d₆ containing trimethylsilane is provided in FIG. 3. The ¹H NMR spectrum was consistent with the structure of the compound of formula (I).

A DSC thermogram for the anhydrous crystalline form of the compound of formula (I) is provided in FIG. 4. The DSC thermogram displayed an endothermic event with an onset value of about 175.5° C. and a peak maximum of about 178.2° C.

A TGA thermogram for the anhydrous crystalline form of the compound of formula (I) is provided in FIG. 4. Negligible weight loss was observed (<0.1% wt.), indicating that the crystalline form is anhydrous.

A water sorption isotherm for the anhydrous crystalline form of the compound of formula (I) is provided in FIG. 5. The data indicates that the anhydrous crystalline form is slightly hygroscopic. During the sorption step, the material exhibited a weight gain of 0.4% from 5 to 95% relative humidity (RH) or 0.1 moles of water per mole of API. The weight gained was lost on desorption with very minor hysteresis.

TABLE 9
XRPD Pattern Data of the Anhydrous Crystalline
Form of the Compound of Formula (I)

| °2θ | d space (Å) | Intensity (%) |
|---|---|---|
| 6.02 ± 0.20 | 14.669 ± 0.487 | 100 |
| 7.09 ± 0.20 | 12.458 ± 0.351 | 6 |
| 8.61 ± 0.20 | 10.262 ± 0.238 | 57 |
| 11.73 ± 0.20 | 7.538 ± 0.128 | 6 |
| 12.08 ± 0.20 | 7.321 ± 0.121 | 4 |
| 14.34 ± 0.20 | 6.172 ± 0.086 | 75 |
| 14.73 ± 0.20 | 6.009 ± 0.081 | 20 |
| 15.08 ± 0.20 | 5.870 ± 0.077 | 9 |
| 15.14 ± 0.20 | 5.847 ± 0.077 | 10 |
| 15.62 ± 0.20 | 5.669 ± 0.072 | 41 |
| 16.33 ± 0.20 | 5.424 ± 0.066 | 64 |
| 17.38 ± 0.20 | 5.098 ± 0.058 | 37 |
| 18.20 ± 0.20 | 4.870 ± 0.053 | 35 |
| 18.47 ± 0.20 | 4.800 ± 0.052 | 25 |
| 19.60 ± 0.20 | 4.526 ± 0.046 | 11 |
| 19.94 ± 0.20 | 4.449 ± 0.044 | 39 |
| 20.35 ± 0.20 | 4.360 ± 0.042 | 29 |
| 20.55 ± 0.20 | 4.318 ± 0.042 | 20 |
| 20.88 ± 0.20 | 4.251 ± 0.040 | 24 |
| 21.48 ± 0.20 | 4.134 ± 0.038 | 46 |
| 22.00 ± 0.20 | 4.037 ± 0.036 | 16 |
| 22.27 ± 0.20 | 3.989 ± 0.035 | 19 |
| 22.68 ± 0.20 | 3.917 ± 0.034 | 18 |
| 22.96 ± 0.20 | 3.870 ± 0.033 | 13 |
| 23.21 ± 0.20 | 3.829 ± 0.033 | 19 |
| 24.37 ± 0.20 | 3.650 ± 0.029 | 26 |
| 24.76 ± 0.20 | 3.593 ± 0.029 | 28 |
| 25.17 ± 0.20 | 3.535 ± 0.028 | 22 |
| 25.57 ± 0.20 | 3.481 ± 0.027 | 10 |
| 26.12 ± 0.20 | 3.409 ± 0.026 | 5 |
| 26.44 ± 0.20 | 3.368 ± 0.025 | 17 |
| 27.13 ± 0.20 | 3.284 ± 0.024 | 8 |
| 27.50 ± 0.20 | 3.241 ± 0.023 | 4 |
| 28.13 ± 0.20 | 3.170 ± 0.022 | 6 |
| 28.53 ± 0.20 | 3.126 ± 0.021 | 8 |
| 28.74 ± 0.20 | 3.104 ± 0.021 | 5 |
| 29.26 ± 0.20 | 3.050 ± 0.020 | 9 |
| 29.79 ± 0.20 | 2.997 ± 0.020 | 7 |
| 30.51 ± 0.20 | 2.928 ± 0.019 | 5 |

A suitable single crystal of the anhydrous crystalline form of the compound of formula (I) was selected and analyzed by single-crystal X-ray diffractometry. A light orange needle having approximate dimensions of 0.27×0.04×0.03 mm³, was mounted on a polymer loop in random orientation. The unit cell parameters of the anhydrous crystalline form and the data collection and structure refinement methods are shown in Table 10.

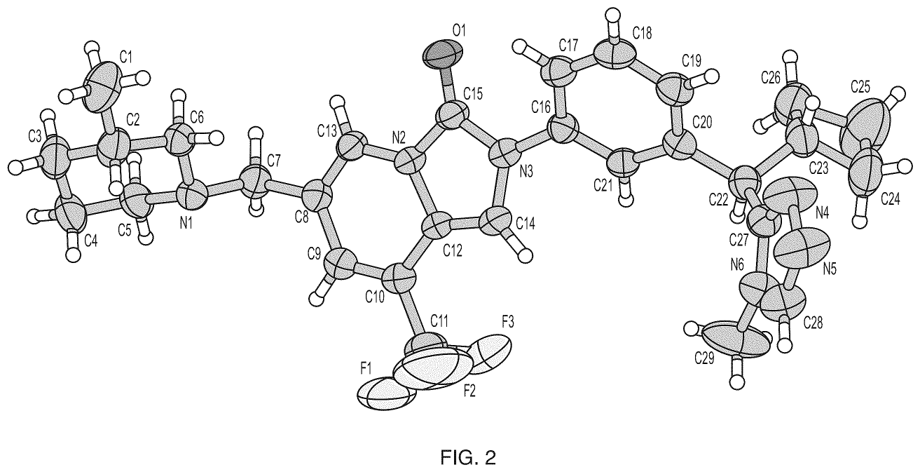

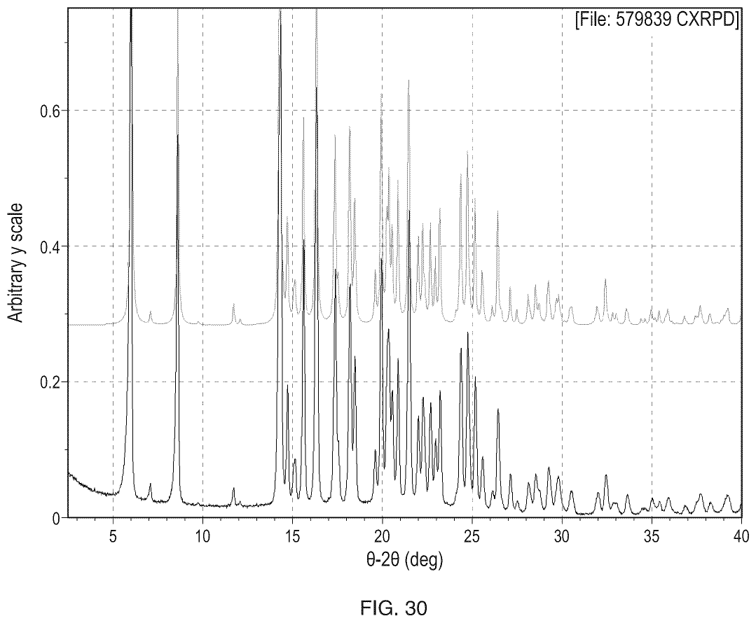

The quality of the structure obtained is high, as indicated by the fit residual, R, of 0.0456 (4.56%). R-factors in the range 2%-6% are quoted to be the most reliably determined structures (Glusker, Jenny Pickworth; Trueblood, Kenneth N. Crystal Structure Analysis: A Primer, 3^{rd} ed.; Oxford University press: New York, 2010; p. 97.). An atomic displacement ellipsoid drawing of the anhydrous crystalline form is shown in FIG. 2. The asymmetric unit contains one molecule of the compound of formula (I) and the chiral centers are S (C2 near the piperidine ring) and R (C22 near the cyclobutane ring). An overlay of a XRPD pattern for the anhydrous crystalline form calculated from the single crystal XRD data and an experimental XRPD pattern is located in FIG. 30. The experimental and calculated XRPD patterns are a good match.

TABLE 10
Crystal Data and Data Collection Parameters of the Anhydrous Crystalline

| | |
|---|---|
| Empirical formula | C₂₉H₃₃F₃N₆O |
| Formula weight (g mol⁻¹) | 538.61 |
| Temperature (K) | 299.9(5) |
| Wavelength (Å) | 1.54184 |
| Crystal system | orthorhombic |
| Space group | P2₁2₁2₁ |
| Unit cell parameters | |
| a = 6.37450(10) Å | α = 90° |
| b = 18.1063(3) Å | β = 90° |
| c = 24.8603(4) Å | γ = 90° |
| Unit cell volume (Å³) | 2869.34(8) |
| Cell formula units, Z | 4 |
| Calculated density (g cm⁻³) | 1.247 |
| Absorption coefficient (mm⁻¹) | 0.762 |
| F(000) | 1136 |
| Crystal size (mm³) | 0.27 × 0.04 × 0.03 |
| Reflections used for cell measurement | 7350 |
| θ range for cell measurement | 4.3060°-75.1220° |
| Total reflections collected | 13966 |
| Index ranges | −7 ≤ h ≤ 6; −22 ≤ k ≤ 22; −15 ≤ l ≤ 30 |
| θ range for data collection | θₘᵢₙ = 3.556°, θₘₐₓ = 75.855° |
| Completeness to θₘₐₓ | 98.5% |
| Completeness to θ_{full} = 67.684° | 100% |
| Absorption correction | multi-scan |
| Transmission coefficient range | 0.951-1.000 |
| Refinement method | full matrix least-squares on F² |
| Independent reflections | 5799 [Rᵢₙₜ = 0.0211, R_{σ} = 0.0262] |
| Reflections [I > 2σ(I)] | 5239 |
| Reflections/restraints/parameters | 5799/0/354 |
| Goodness-of-fit on F² | S = 1.05 |
| Final residuals [I > 2σ(I)] | R = 0.0456, R_{w} = 0.1272 |
| Final residuals [all reflections] | R = 0.0503, R_{w} = 0.1315 |
| Largest diff. peak and hole (e Å⁻³) | 0.315, −0.255 |
| Max/mean shift/standard uncertainty | 0.000/0.000 |
| Absolute structure determination | Flack parameter: 0.03(8) |

(iii) Solubility Determination

Aliquots of various solvents were added to measured amounts of the anhydrous crystalline form of the compound of formula (I) at various temperatures with sonication or stirring. Solubilities were calculated based on the total solvent used to give a solution; actual solubilities may be greater because of the volume of solvent portions utilized or a slow rate of dissolution. If dissolution did not occur as determined by visual assessment value was reported as “<”. If dissolution occurred at the first aliquot the value was reported as “>”.

The solubility of the anhydrous crystalline form in various solvents is given in Table 11.

TABLE 11
Solubility of the Anhydrous Crystalline Form of the
Compound of Formula (I) in Various Solvent Systems

| Solvent | Solubility (mg/ml) |
|---|---|
| Acetone | 31 |
| ACN | 85 |
| DCM | >100 |
| DIPE | <2 |
| EtOAc | 38 |
| EtOH | 99 |
| Heptane | <1 |
| IPA | 79 |
| MeOH | 91 |
| MTBE | 4 |
| THF | 95 |
| Toluene | 99 |
| Water | <1 |
| Water/IPA | >30 |
| (20/80 v/v) | |
| Water/IPA | >37 |
| (40/60 v/v) | |
| Water/IPA | 9 |
| (60/40 v/v) | |
| Water/EtOH | >29 |
| (20/80 v/v) | |
| Water/EtOH | >36 |
| (40/60 v/v) | |
| Water/EtOH | 9 |
| (50/50 v/v) | |
| Water/EtOH | <4 |
| (60/40 v/v) | |
| Water/MeOH | >29 |
| (20/80 v/v) | |
| Water/MeOH | 7 |
| (40/60 v/v) | |
| Water/MeOH | <3 |
| (60/40 v/v) | |

### Example 3—Crystalline Acetone Solvate of the Compound of Formula (I)

### (i) Preparation of the Crystalline Acetone Solvate

The crystalline acetone solvate of the compound of formula (I) was prepared as follows: 161 mg of the anhydrous crystalline form of the compound of formula (I) (see Example 2) was stirred in acetone (0.7 ml) at 55° C. The resulting suspension was then stirred at ambient temperature. The solids were isolated by syringe filtration after 6 days.

### (ii) Characterization of the Crystalline Acetone Solvate

An XRPD pattern for the crystalline acetone solvate of the compound of formula (I) is provided in FIG. 6. Tabulated characteristics of the XRPD pattern in FIG. 6 are provided in Table 12, which lists diffraction angle 2θ, d-spacing [Å], and relative intensity (expressed as a percentage with respect to the most intense peak).

An ¹H NMR spectrum for the crystalline acetone solvate of the compound of formula (I) dissolved in dimethylsulfoxide-d₆ containing trimethylsilane is provided in FIG. 8. The ¹H NMR spectrum was consistent with the structure of the compound of formula (I) and contained ˜1 mole of acetone.

A DSC thermogram for the crystalline acetone solvate of the compound of formula (I) is provided in FIG. 9. The DSC thermogram displayed two endothermic events: (1) onset value of about 89.9° C. and a peak maximum of about 100.1° C. and (2) a peak maximum of about 173.1° C.

A TGA thermogram for the crystalline acetone solvate of the compound of formula (I) is provided in FIG. 9. The data indicated a 5.3% wt. loss from 61 to 121° C. corresponding to about 0.5 moles of acetone.

TABLE 12
XRPD Pattern Data of the Crystalline Acetone
Solvate of the Compound of Formula (I)

| °2θ | d space (Å) | Intensity (%) |
|---|---|---|
| 6.40 ± 0.20 | 13.799 ± 0.431 | 100 |
| 7.83 ± 0.20 | 11.282 ± 0.288 | 6 |
| 9.76 ± 0.20 | 9.055 ± 0.185 | 4 |
| 10.42 ± 0.20 | 8.483 ± 0.162 | 4 |
| 11.97 ± 0.20 | 7.388 ± 0.123 | 28 |
| 12.83 ± 0.20 | 6.894 ± 0.107 | 4 |
| 14.12 ± 0.20 | 6.267 ± 0.088 | 5 |
| 14.31 ± 0.20 | 6.184 ± 0.086 | 21 |
| 15.09 ± 0.20 | 5.866 ± 0.077 | 3 |
| 15.70 ± 0.20 | 5.640 ± 0.071 | 14 |
| 16.19 ± 0.20 | 5.470 ± 0.067 | 42 |
| 17.23 ± 0.20 | 5.142 ± 0.059 | 35 |
| 17.60 ± 0.20 | 5.035 ± 0.057 | 8 |
| 17.82 ± 0.20 | 4.973 ± 0.055 | 19 |
| 18.39 ± 0.20 | 4.821 ± 0.052 | 10 |
| 18.60 ± 0.20 | 4.767 ± 0.051 | 7 |
| 19.17 ± 0.20 | 4.626 ± 0.048 | 5 |
| 20.18 ± 0.20 | 4.397 ± 0.043 | 25 |
| 20.52 ± 0.20 | 4.325 ± 0.042 | 6 |
| 21.21 ± 0.20 | 4.186 ± 0.039 | 12 |
| 21.79 ± 0.20 | 4.075 ± 0.037 | 29 |
| 21.99 ± 0.20 | 4.039 ± 0.036 | 43 |
| 22.64 ± 0.20 | 3.924 ± 0.034 | 25 |
| 22.83 ± 0.20 | 3.892 ± 0.034 | 13 |
| 23.28 ± 0.20 | 3.818 ± 0.032 | 11 |
| 23.58 ± 0.20 | 3.770 ± 0.032 | 14 |
| 24.00 ± 0.20 | 3.705 ± 0.030 | 8 |
| 24.28 ± 0.20 | 3.663 ± 0.030 | 9 |
| 24.88 ± 0.20 | 3.576 ± 0.028 | 2 |
| 25.01 ± 0.20 | 3.557 ± 0.028 | 2 |
| 25.37 ± 0.20 | 3.508 ± 0.027 | 4 |
| 25.84 ± 0.20 | 3.445 ± 0.026 | 7 |
| 26.08 ± 0.20 | 3.414 ± 0.026 | 6 |
| 26.25 ± 0.20 | 3.392 ± 0.025 | 3 |
| 27.04 ± 0.20 | 3.295 ± 0.024 | 2 |
| 27.70 ± 0.20 | 3.218 ± 0.023 | 9 |
| 28.26 ± 0.20 | 3.155 ± 0.022 | 6 |
| 28.68 ± 0.20 | 3.110 ± 0.021 | 3 |
| 29.29 ± 0.20 | 3.047 ± 0.020 | 2 |
| 29.69 ± 0.20 | 3.007 ± 0.020 | 2 |
| 30.11 ± 0.20 | 2.966 ± 0.019 | 4 |
| 30.57 ± 0.20 | 2.922 ± 0.019 | 2 |
| 31.06 ± 0.20 | 2.877 ± 0.018 | 4 |

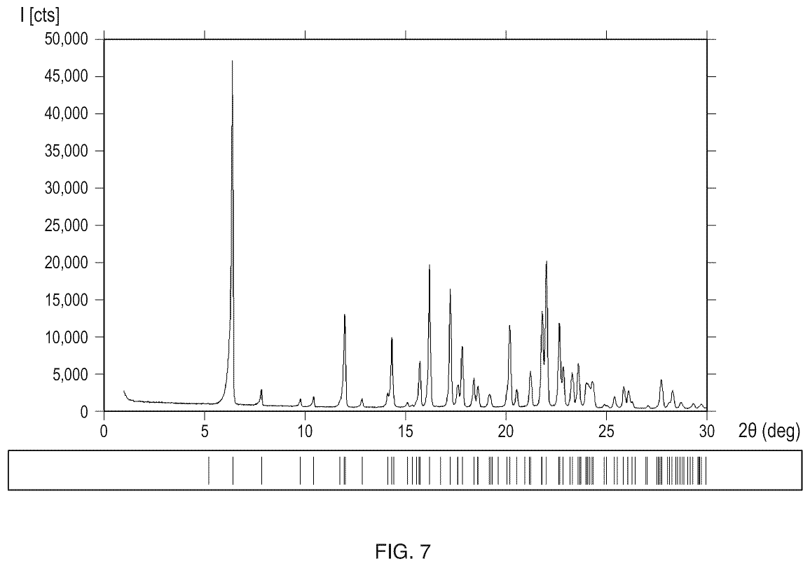

The XRPD pattern for the crystalline acetone solvate of the compound of formula (I) was successfully indexed (FIG. 7). The volume from the unit cell indicated that it is large enough to contain 1 mole of acetone per molecule of the compound of formula (I) (Table 13).

TABLE 13
Unit Cell Parameters of the Crystalline Acetone
Solvate of the Compound of Formula (I)

| | Primitive |
|---|---|
| Bravais Type | Orthorhombic |
| a [Å] | 6.251 |
| b [Å] | 15.082 |
| c [Å] | 33.920 |
| α [deg] | 90 |
| β [deg] | 90 |
| γ [deg] | 90 |
| Volume [Å³/cell] | 3197.9 |
| Chiral Contents? | Chiral |
| Extinction Symbol | P 2₁ 2₁ 2₁ |
| Space Group(s) | P2₁2₁2₁ (19) |

### Example 4—Crystalline p-Dioxane Solvate of the Compound of Formula (I)

(i) Preparation of the Crystalline p-Dioxane Solvate

The crystalline p-dioxane solvate of the compound of formula (I) was prepared as follows: 160 mg of the anhydrous crystalline form of the compound of formula (I) (see Example 2) was stirred at 55° C. in a mixture of heptane (0.5 ml) and p-dioxane (0.5 ml). The resulting suspension was then stirred at ambient temperature for 6 days. The solids were isolated by syringe filtration.

Alternative method: 74 mg of the compound of formula (I) was dissolved in heptane (0.5 ml) and p-dioxane (0.5 ml) at 60° C. The solution was filtered through a 0.2 μm nylon filter into a pre-warmed (60° C.) vial. The vial was capped and the sample was cooled to ambient temperature at 6° C./h. Solids were isolated by syringe filtration.

(ii) Characterization of the Crystalline p-Dioxane Solvate

An XRPD pattern for the crystalline p-dioxane solvate of the compound of formula (I) is provided in FIG. 10. Tabulated characteristics of the XRPD pattern in FIG. 10 are provided in Table 14, which lists diffraction angle 2θ, d-spacing [Å], and relative intensity (expressed as a percentage with respect to the most intense peak).

An ¹H NMR spectrum for the crystalline p-dioxane solvate of the compound of formula (I) dissolved in dimethylsulfoxide-d₆ containing trimethylsilane is provided in FIG. 12. The ¹H NMR spectrum was consistent with the structure of the compound of formula (I) and contained ˜0.9 mole of p-dioxane.

A DSC thermogram for the crystalline p-dioxane solvate of the compound of formula (I) is provided in FIG. 13. The DSC thermogram displayed a single endothermic event with an onset value of about 94.2° C. and a peak maximum of about 106.3° C.

A TGA thermogram for the crystalline p-dioxane solvate of the compound of formula (I) is provided in FIG. 13. The data indicated a 3.9% wt. loss from 51 to 120° C. or 0.2 moles of p-dioxane.

TABLE 14
XRPD Pattern Data of the Crystalline p-Dioxane
Solvate of the Compound of Formula (I)

| °2θ | d space (Å) | Intensity (%) |
|---|---|---|
| 5.16 ± 0.20 | 17.112 ± 0.663 | 11 |
| 6.29 ± 0.20 | 14.040 ± 0.446 | 66 |
| 7.73 ± 0.20 | 11.428 ± 0.295 | 8 |
| 10.34 ± 0.20 | 8.548 ± 0.165 | 5 |
| 11.84 ± 0.20 | 7.468 ± 0.126 | 21 |
| 12.62 ± 0.20 | 7.009 ± 0.111 | 4 |
| 14.17 ± 0.20 | 6.245 ± 0.088 | 31 |
| 14.93 ± 0.20 | 5.929 ± 0.079 | 5 |
| 15.38 ± 0.20 | 5.757 ± 0.074 | 8 |
| 15.50 ± 0.20 | 5.712 ± 0.073 | 11 |
| 16.02 ± 0.20 | 5.528 ± 0.069 | 100 |
| 17.04 ± 0.20 | 5.199 ± 0.061 | 35 |
| 17.49 ± 0.20 | 5.067 ± 0.057 | 20 |
| 18.07 ± 0.20 | 4.905 ± 0.054 | 10 |
| 18.37 ± 0.20 | 4.826 ± 0.052 | 10 |
| 18.90 ± 0.20 | 4.692 ± 0.049 | 9 |
| 19.06 ± 0.20 | 4.653 ± 0.048 | 10 |
| 19.40 ± 0.20 | 4.572 ± 0.047 | 5 |
| 19.98 ± 0.20 | 4.440 ± 0.044 | 19 |
| 20.20 ± 0.20 | 4.392 ± 0.043 | 9 |
| 20.95 ± 0.20 | 4.237 ± 0.040 | 15 |
| 21.58 ± 0.20 | 4.115 ± 0.038 | 29 |
| 21.78 ± 0.20 | 4.077 ± 0.037 | 60 |
| 22.39 ± 0.20 | 3.968 ± 0.035 | 15 |
| 22.95 ± 0.20 | 3.872 ± 0.033 | 14 |
| 23.14 ± 0.20 | 3.841 ± 0.033 | 13 |
| 23.80 ± 0.20 | 3.736 ± 0.031 | 12 |
| 24.01 ± 0.20 | 3.703 ± 0.030 | 18 |
| 24.67 ± 0.20 | 3.606 ± 0.029 | 4 |
| 25.16 ± 0.20 | 3.537 ± 0.028 | 8 |
| 25.42 ± 0.20 | 3.501 ± 0.027 | 7 |
| 25.82 ± 0.20 | 3.448 ± 0.026 | 7 |
| 27.33 ± 0.20 | 3.261 ± 0.023 | 7 |
| 27.75 ± 0.20 | 3.212 ± 0.023 | 5 |
| 28.02 ± 0.20 | 3.182 ± 0.022 | 7 |
| 28.27 ± 0.20 | 3.154 ± 0.022 | 5 |
| 28.94 ± 0.20 | 3.083 ± 0.021 | 5 |

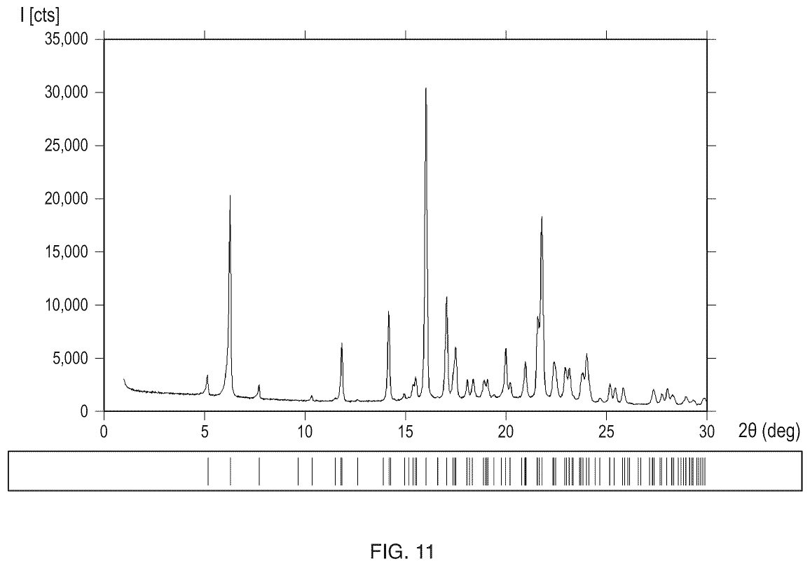

The XRPD pattern for the crystalline p-dioxane solvate of the compound of formula (I) was successfully indexed (FIG. 11). The volume from the unit cell indicated that it could contain 1 mole of p-dioxane per mole of the compound of formula (I) (Table 15).

TABLE 15
Unit Cell Parameters of the Crystalline p-Dioxane
Solvate of the Compound of Formula (I)

| | Primitive |
|---|---|
| Bravais Type | Orthorhombic |
| a [Å] | 6.316 |
| b [Å] | 15.364 |
| c [Å] | 34.166 |
| α [deg] | 90 |
| β [deg] | 90 |
| γ [deg] | 90 |
| Volume [Å³/cell] | 3315.4 |
| Chiral Contents? | Chiral |
| Extinction Symbol | P 2₁ 2₁ 2₁ |
| Space Group(s) | P2₁2₁2₁ (19) |

### Example 5—Crystalline THF Solvate of the Compound of Formula (I)

### (i) Preparation of the Crystalline THF Solvate

The crystalline THF solvate of the compound of formula (I) was prepared as follows: 144 mg of the anhydrous crystalline form of the compound of formula (I) (see Example 2) was stirred in heptane (0.5 ml) and tetrahydrofuran (0.5 ml) at 55° C. The resulting suspension was stirred at ambient temperature. The solids were isolated after 6 days by syringe filtration.

Alternative method: 51 mg of the compound of formula (I) was dissolved at 60° C. in a mixture of cyclohexane (0.5 ml) and tetrahydrofuran (0.4 ml). The solution was filtered through a 0.2 μm nylon filter into a pre-warmed (60° C.) vial. The sample was then cooled to ambient temperature at 6° C./h. The solids were isolated by syringe filtration.

### (ii) Characterization of the Crystalline THF Solvate

An XRPD pattern for the crystalline THF solvate of the compound of formula (I) is provided in FIG. 14. Tabulated characteristics of the XRPD pattern in FIG. 14 are provided in Table 16, which lists diffraction angle 2θ, d-spacing [Å], and relative intensity (expressed as a percentage with respect to the most intense peak).

An ¹H NMR spectrum for the crystalline THF solvate of the compound of formula (I) dissolved in dimethylsulfoxide-d₆ containing trimethylsilane is provided in FIG. 16. The ¹H NMR spectrum was consistent with the structure of the compound of formula (I) and contained ˜0.7 mole of THF and 0.1 moles of cyclohexane.

A DSC thermogram for the crystalline THF solvate of the compound of formula (I) is provided in FIG. 17. The DSC thermogram displayed a single endothermic event with an onset value of about 84.8° C. and a peak maximum of about 97.5° C.

A TGA thermogram for the crystalline THF solvate of the compound of formula (I) is provided in FIG. 17. The data indicated 4.5% wt. loss from 56 to 112° C. or 0.4 moles of THF.

TABLE 16
XRPD Pattern Data of the Crystalline THF
Solvate of the Compound of Formula (I)

| °2θ | d space (Å) | Intensity (%) |
|---|---|---|
| 6.25 ± 0.20 | 14.130 ± 0.452 | 100 |
| 7.71 ± 0.20 | 11.457 ± 0.297 | 5 |
| 11.39 ± 0.20 | 7.763 ± 0.136 | 5 |
| 11.69 ± 0.20 | 7.564 ± 0.129 | 9 |
| 11.90 ± 0.20 | 7.431 ± 0.124 | 6 |
| 12.54 ± 0.20 | 7.053 ± 0.112 | 5 |
| 14.27 ± 0.20 | 6.202 ± 0.086 | 6 |
| 15.18 ± 0.20 | 5.832 ± 0.076 | 6 |
| 15.45 ± 0.20 | 5.731 ± 0.074 | 8 |
| 16.07 ± 0.20 | 5.511 ± 0.068 | 42 |
| 17.11 ± 0.20 | 5.178 ± 0.060 | 21 |
| 17.34 ± 0.20 | 5.110 ± 0.058 | 45 |
| 17.93 ± 0.20 | 4.943 ± 0.055 | 15 |
| 18.33 ± 0.20 | 4.836 ± 0.052 | 9 |
| 18.89 ± 0.20 | 4.694 ± 0.049 | 8 |
| 19.80 ± 0.20 | 4.480 ± 0.045 | 5 |
| 20.11 ± 0.20 | 4.412 ± 0.043 | 11 |
| 20.99 ± 0.20 | 4.229 ± 0.040 | 9 |
| 21.65 ± 0.20 | 4.101 ± 0.037 | 9 |
| 21.93 ± 0.20 | 4.050 ± 0.036 | 11 |
| 22.41 ± 0.20 | 3.964 ± 0.035 | 26 |
| 22.91 ± 0.20 | 3.879 ± 0.033 | 44 |
| 23.52 ± 0.20 | 3.779 ± 0.032 | 11 |
| 24.09 ± 0.20 | 3.691 ± 0.030 | 6 |
| 24.60 ± 0.20 | 3.615 ± 0.029 | 3 |
| 25.26 ± 0.20 | 3.523 ± 0.027 | 7 |
| 25.94 ± 0.20 | 3.432 ± 0.026 | 3 |
| 27.13 ± 0.20 | 3.284 ± 0.024 | 5 |
| 27.34 ± 0.20 | 3.259 ± 0.023 | 4 |
| 28.43 ± 0.20 | 3.136 ± 0.022 | 5 |
| 28.92 ± 0.20 | 3.085 ± 0.021 | 5 |
| 29.46 ± 0.20 | 3.029 ± 0.020 | 3 |
| 30.00 ± 0.20 | 2.976 ± 0.019 | 3 |
| 30.78 ± 0.20 | 2.903 ± 0.018 | 4 |

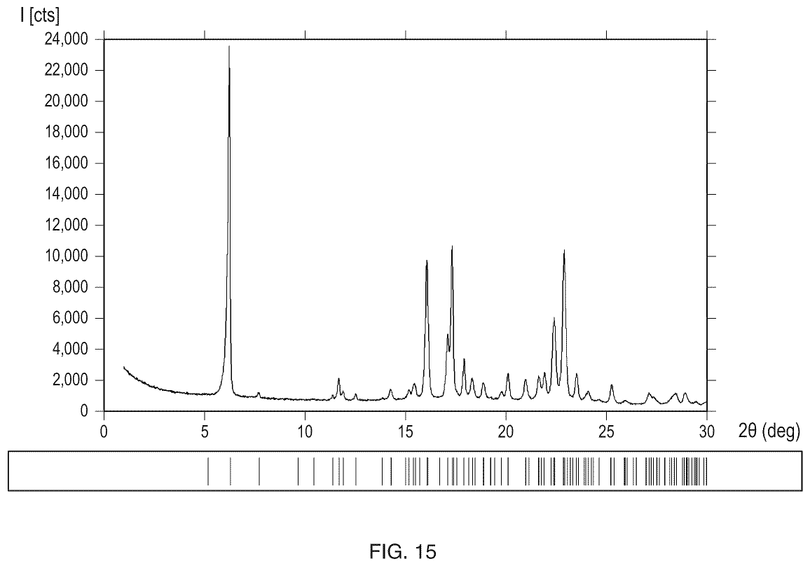

The XRPD pattern for the crystalline THF solvate of the compound of formula (I) was successfully indexed (FIG. 15). The volume from the unit cell indicated that it could contain 1 mole of THF per mole of compound of formula (I) (Table 17).

TABLE 17
Unit Cell Parameters of the Crystalline THF
Solvate of the Compound of Formula (I)

| | Primitive |
|---|---|
| Bravais Type | Orthorhombic |
| a [Å] | 6.289 |
| b [Å] | 15.515 |
| c [Å] | 33.823 |
| α [deg] | 90 |
| β [deg] | 90 |
| γ [deg] | 90 |
| Volume [Å³/cell] | 3300.2 |
| Chiral Contents? | Chiral |
| Extinction Symbol | P 2₁ 2₁ 2₁ |
| Space Group(s) | P2₁2₁2₁ (19) |

### Example 6—Acetone Solvated Crystalline Citrate Salt of the Compound of Formula (I)

### (i) Preparation of the Acetone Solvated Crystalline Citrate Salt

The acetone solvated crystalline citrate salt of the compound of formula (I) was prepared as follows: an amorphous form of the compound of formula (I) was prepared via column chromatography of the compound of formula (I), followed by evaporation. Then 71 mg of the amorphous solid of the compound of formula (I) and 1 molar equivalent of citric acid (25 mg) were stirred in acetone (0.5 ml) at ambient temperature for 1 day. A thick slurry resulted and additional acetone (0.5 ml) was added. The mixture was stirred at ambient temperature for 3 additional days. The solids were isolated by syringe filtration using a Swinnex filtration assembly.

### (ii) Characterization of the Acetone Solvated Crystalline Citrate Salt

An XRPD pattern for the acetone solvated crystalline citrate salt of the compound of formula (I) is provided in FIG. 18. Tabulated characteristics of the XRPD pattern in FIG. 18 are provided in Table 18, which lists diffraction angle 2θ, d-spacing [Å], and relative intensity (expressed as a percentage with respect to the most intense peak).

An ¹H NMR spectrum for the acetone solvated crystalline citrate salt of the compound of formula (I) dissolved in dimethylsulfoxide-d₆ containing trimethylsilane is provided in FIG. 20. The ¹H NMR spectrum was consistent with the structure of the compound of formula (I) and approximately 1 mole of citric acid. Approximately 2 moles of acetone were also observed in the spectrum, however, it is noted that peak overlap of acetone and the compound of formula (I) was observed and the actual amount of acetone may be less than 2 moles.

A DSC thermogram for the acetone solvated crystalline citrate salt of the compound of formula (I) is provided in FIG. 21. The DSC thermogram displayed a single endothermic event with an onset value of about 109.8° C. and a peak maximum of about 117.4° C.

A TGA thermogram for the acetone solvated crystalline citrate salt of the compound of formula (I) is provided in FIG. 21. The data indicated 7.0% weight loss from 46 to 131° C. and is likely due to acetone (0.9 moles).

TABLE 18
XRPD Pattern Data of the Acetone Solvated Crystalline
Citrate Salt of the Compound of Formula (I)

| °2θ | d space (Å) | Intensity (%) |
|---|---|---|
| 4.99 ± 0.20 | 17.695 ± 0.709 | 69 |
| 6.64 ± 0.20 | 13.301 ± 0.400 | 100 |
| 8.61 ± 0.20 | 10.262 ± 0.238 | 8 |
| 10.01 ± 0.20 | 8.829 ± 0.176 | 10 |
| 11.02 ± 0.20 | 8.022 ± 0.145 | 9 |
| 11.50 ± 0.20 | 7.689 ± 0.133 | 5 |
| 13.20 ± 0.20 | 6.702 ± 0.101 | 8 |
| 13.32 ± 0.20 | 6.642 ± 0.099 | 5 |
| 14.61 ± 0.20 | 6.058 ± 0.082 | 8 |
| 15.03 ± 0.20 | 5.890 ± 0.078 | 17 |
| 15.25 ± 0.20 | 5.805 ± 0.076 | 14 |
| 15.85 ± 0.20 | 5.587 ± 0.070 | 9 |
| 16.30 ± 0.20 | 5.434 ± 0.066 | 4 |
| 16.49 ± 0.20 | 5.371 ± 0.065 | 4 |
| 16.87 ± 0.20 | 5.251 ± 0.062 | 19 |
| 17.56 ± 0.20 | 5.046 ± 0.057 | 54 |
| 18.17 ± 0.20 | 4.878 ± 0.053 | 55 |
| 19.71 ± 0.20 | 4.501 ± 0.045 | 15 |
| 20.09 ± 0.20 | 4.416 ± 0.044 | 27 |
| 21.04 ± 0.20 | 4.219 ± 0.040 | 10 |
| 21.52 ± 0.20 | 4.126 ± 0.038 | 7 |
| 21.84 ± 0.20 | 4.066 ± 0.037 | 4 |
| 22.59 ± 0.20 | 3.933 ± 0.034 | 17 |
| 22.76 ± 0.20 | 3.904 ± 0.034 | 15 |
| 23.16 ± 0.20 | 3.837 ± 0.033 | 12 |
| 23.38 ± 0.20 | 3.802 ± 0.032 | 13 |
| 23.88 ± 0.20 | 3.723 ± 0.031 | 4 |
| 24.61 ± 0.20 | 3.614 ± 0.029 | 12 |
| 24.81 ± 0.20 | 3.586 ± 0.028 | 33 |
| 25.20 ± 0.20 | 3.532 ± 0.028 | 5 |
| 26.00 ± 0.20 | 3.424 ± 0.026 | 6 |
| 26.59 ± 0.20 | 3.350 ± 0.025 | 5 |
| 27.26 ± 0.20 | 3.269 ± 0.024 | 11 |
| 28.94 ± 0.20 | 3.083 ± 0.021 | 5 |
| 29.45 ± 0.20 | 3.031 ± 0.020 | 4 |
| 29.82 ± 0.20 | 2.994 ± 0.020 | 4 |
| 30.39 ± 0.20 | 2.939 ± 0.019 | 7 |

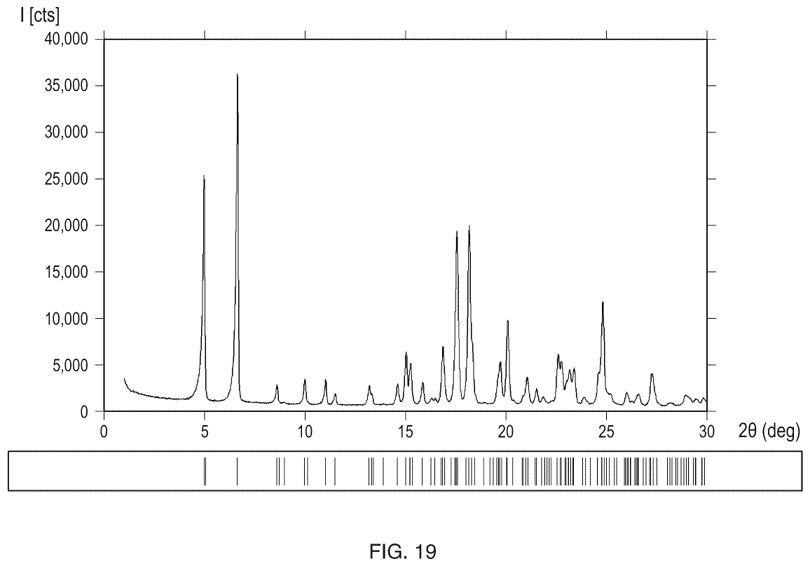

The XRPD pattern for the acetone solvated crystalline citrate salt of the compound of formula (I) was successfully indexed (FIG. 19). The volume from the unit cell indicated a solvated mono-citrate salt. (Table 19).

TABLE 19
Unit Cell Parameters of the Acetone Solvated Crystalline
Citrate Salt of the Compound of Formula (I)

| | Primitive |
|---|---|
| Bravais Type | Orthorhombic |
| a [Å] | 6.150 |
| b [Å] | 20.489 |
| c [Å] | 34.845 |
| α [deg] | 90 |
| β [deg] | 90 |
| γ [deg] | 90 |
| Volume [Å³/cell] | 4390.7 |
| Chiral Contents? | Chiral |
| Extinction Symbol | P 2₁ 2₁ 2₁ |
| Space Group(s) | P2₁2₁2₁ (19) |

### Example 7—ACN Solvated Crystalline Citrate Salt of the Compound of Formula (I)

### (i) Preparation of the ACN Solvated Crystalline Citrate Salt

The ACN solvated crystalline citrate salt of the compound of formula (I) was prepared as follows: 42 mg of the anhydrous crystalline form of the compound of formula (I) (see Example 2) and 0.75 molar equivalent of citric acid (20 mg) were stirred in acetonitrile (2.5 ml) at ambient temperature for approximately 2 weeks. Solids were isolated by syringe filtration using a Swinnex filtration assembly.

### (ii) Characterization of the ACN Solvated Crystalline Citrate Salt

An XRPD pattern for the ACN solvated crystalline citrate salt of the compound of formula (I) is provided in FIG. 22. Tabulated characteristics of the XRPD pattern in FIG. 22 are provided in Table 20, which lists diffraction angle 2θ, d-spacing [Å], and relative intensity (expressed as a percentage with respect to the most intense peak).

TABLE 20
XRPD Pattern Data of the ACN Solvated Crystalline
Citrate Salt of the Compound of Formula (I)

| °2θ | d space (Å) | Intensity (%) |
|---|---|---|
| 5.06 ± 0.20 | 17.450 ± 0.689 | 100 |
| 5.40 ± 0.20 | 16.352 ± 0.605 | 13 |
| 6.89 ± 0.20 | 12.819 ± 0.372 | 53 |
| 8.56 ± 0.20 | 10.321 ± 0.241 | 6 |
| 9.18 ± 0.20 | 9.626 ± 0.209 | 8 |
| 10.14 ± 0.20 | 8.716 ± 0.171 | 10 |
| 10.83 ± 0.20 | 8.163 ± 0.150 | 11 |
| 11.66 ± 0.20 | 7.583 ± 0.130 | 6 |
| 13.16 ± 0.20 | 6.722 ± 0.102 | 5 |
| 13.85 ± 0.20 | 6.389 ± 0.092 | 8 |
| 13.98 ± 0.20 | 6.330 ± 0.090 | 10 |
| 14.79 ± 0.20 | 5.985 ± 0.080 | 17 |
| 15.18 ± 0.20 | 5.832 ± 0.076 | 12 |
| 15.42 ± 0.20 | 5.742 ± 0.074 | 13 |
| 16.11 ± 0.20 | 5.497 ± 0.068 | 17 |
| 16.86 ± 0.20 | 5.254 ± 0.062 | 10 |
| 17.22 ± 0.20 | 5.145 ± 0.059 | 39 |
| 17.42 ± 0.20 | 5.087 ± 0.058 | 15 |
| 17.77 ± 0.20 | 4.987 ± 0.056 | 51 |
| 18.03 ± 0.20 | 4.915 ± 0.054 | 8 |
| 18.71 ± 0.20 | 4.739 ± 0.050 | 44 |
| 19.05 ± 0.20 | 4.655 ± 0.048 | 10 |
| 19.53 ± 0.20 | 4.542 ± 0.046 | 11 |
| 19.67 ± 0.20 | 4.510 ± 0.045 | 12 |
| 19.93 ± 0.20 | 4.451 ± 0.044 | 12 |
| 20.15 ± 0.20 | 4.403 ± 0.043 | 17 |
| 20.39 ± 0.20 | 4.352 ± 0.042 | 21 |
| 20.90 ± 0.20 | 4.247 ± 0.040 | 10 |
| 21.14 ± 0.20 | 4.199 ± 0.039 | 11 |
| 21.75 ± 0.20 | 4.083 ± 0.037 | 14 |
| 21.93 ± 0.20 | 4.050 ± 0.036 | 12 |
| 22.34 ± 0.20 | 3.976 ± 0.035 | 28 |
| 22.61 ± 0.20 | 3.929 ± 0.034 | 13 |
| 22.77 ± 0.20 | 3.902 ± 0.034 | 14 |
| 23.27 ± 0.20 | 3.819 ± 0.032 | 31 |
| 23.57 ± 0.20 | 3.772 ± 0.032 | 27 |
| 23.80 ± 0.20 | 3.736 ± 0.031 | 17 |
| 24.06 ± 0.20 | 3.696 ± 0.030 | 19 |
| 24.43 ± 0.20 | 3.641 ± 0.029 | 23 |
| 25.15 ± 0.20 | 3.538 ± 0.028 | 11 |
| 25.52 ± 0.20 | 3.488 ± 0.027 | 17 |
| 25.95 ± 0.20 | 3.431 ± 0.026 | 12 |
| 26.46 ± 0.20 | 3.366 ± 0.025 | 19 |
| 26.68 ± 0.20 | 3.339 ± 0.025 | 13 |
| 27.38 ± 0.20 | 3.255 ± 0.023 | 11 |
| 27.86 ± 0.20 | 3.200 ± 0.023 | 9 |
| 28.35 ± 0.20 | 3.146 ± 0.022 | 12 |

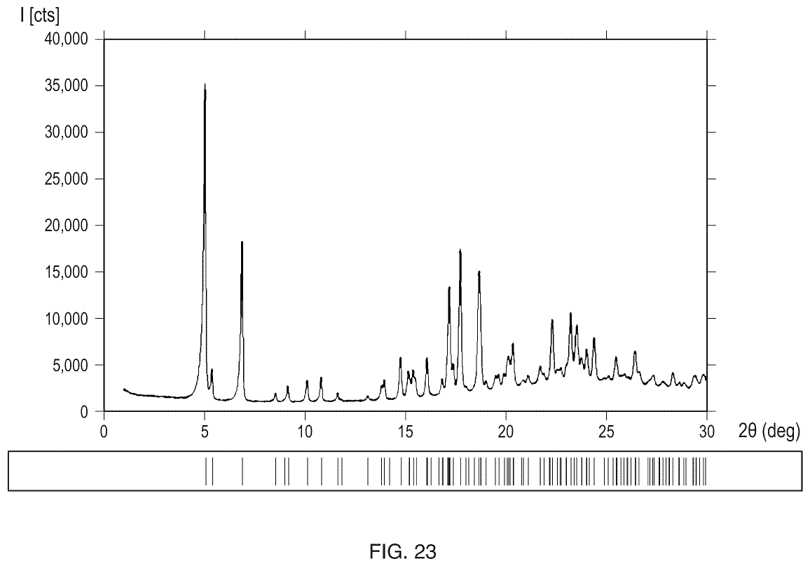

The XRPD pattern for the ACN solvated crystalline citrate salt of the compound of formula (I) was successfully indexed (FIG. 23). The volume from the unit cell indicated a solvated mono-citrate salt. (Table 21).

TABLE 21
Unit Cell Parameters of the ACN Solvated Crystalline
Citrate Salt of the Compound of Formula (I)

| | Primitive |
|---|---|
| Bravais Type | Orthorhombic |
| a [Å] | 6.081 |
| b [Å] | 20.605 |
| c [Å] | 32.628 |
| α [deg] | 90 |
| β [deg] | 90 |
| γ [deg] | 90 |
| Volume [Å³/cell] | 4088.3 |
| Chiral Contents? | Chiral |
| Extinction Symbol | P 2₁ 2₁ 2₁ |
| Space Group(s) | P2₁2₁2₁ (19) |

### Example 8—Anhydrous Crystalline Citrate Salt of the Compound of Formula (I)

### (i) Preparation of the Anhydrous Crystalline Citrate Salt

The anhydrous crystalline citrate salt of the compound of formula (I) was prepared as follows: the ACN solvated crystalline citrate salt of the compound of formula (I) (see Example 7) was placed in a vial and capped with perforated aluminium foil. The sample was then placed in a vacuum oven at ambient temperature for 1 day.

Alternative method: the ACN solvated crystalline citrate salt of the compound of formula (I) (see Example 7) was placed in a vial and capped with perforated aluminium foil. The sample was then placed in a vacuum oven at 44° C. for 1 day.

### (ii) Characterization of the Anhydrous Crystalline Citrate Salt

An XRPD pattern for the anhydrous crystalline citrate salt of the compound of formula (I) is provided in FIG. 24. Tabulated characteristics of the XRPD pattern in FIG. 24 are provided in Table 22, which lists diffraction angle 2θ, d-spacing [Å], and relative intensity (expressed as a percentage with respect to the most intense peak).

An ¹H NMR spectrum for the anhydrous crystalline form of the compound of formula (I) dissolved in dimethylsulfoxide-d₆ containing trimethylsilane is provided in FIG. 26. The ¹H NMR spectrum was consistent with the structure of the compound of formula (I) with approximately 1 mole of citric acid.

A DSC thermogram for the anhydrous crystalline form of the compound of formula (I) is provided in FIG. 28. The DSC thermogram displayed an endothermic event with a peak maximum of about 131.5° C. A second endotherm was observed immediately after this event and is likely due to decomposition.

A TGA thermogram for the anhydrous crystalline form of the compound of formula (I) is provided in FIG. 27. Negligible weight loss was observed (<0.1% wt.), indicating that the crystalline form is anhydrous.

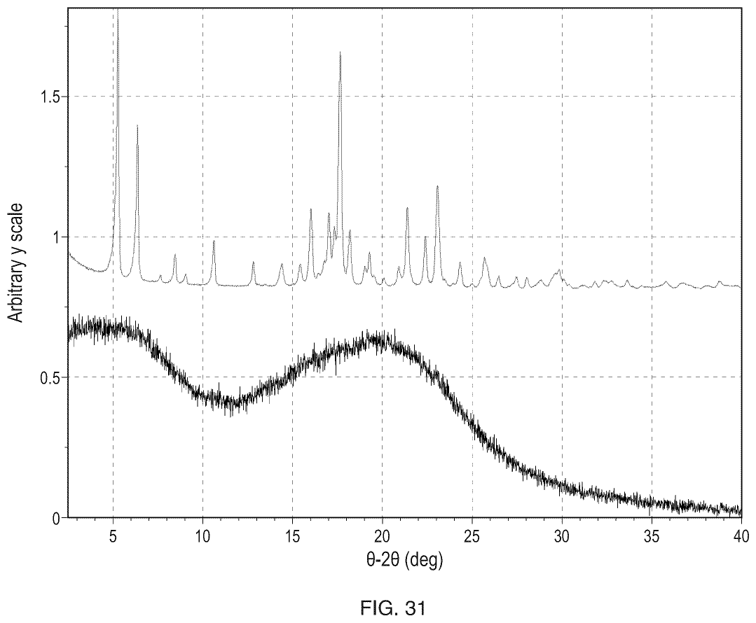

A water sorption isotherm for the anhydrous crystalline form of the compound of formula (I) is provided in FIG. 29. The isotherm exhibited a weight gain of 1.2% wt. from 5% to 74% RH and 14.6% wt. from 75% to 96% RH. The total weight gained was 15.8% or 7.6 moles of water. Significant hysteresis was observed upon desorption and the sample exhibited signs of partial deliquescence. XRPD analysis on the post-DVS sample indicated the material was amorphous (FIG. 31).

TABLE 22
XRPD Pattern Data of the Anhydrous Crystalline
Citrate Salt of the Compound of Formula (I)

| °2θ | d space (Å) | Intensity (%) |
|---|---|---|
| 5.29 ± 0.20 | 16.692 ± 0.631 | 100 |
| 6.37 ± 0.20 | 13.864 ± 0.435 | 58 |
| 7.64 ± 0.20 | 11.562 ± 0.302 | 12 |
| 8.47 ± 0.20 | 10.431 ± 0.246 | 19 |
| 9.05 ± 0.20 | 9.764 ± 0.215 | 12 |
| 10.61 ± 0.20 | 8.331 ± 0.157 | 22 |
| 12.81 ± 0.20 | 6.905 ± 0.107 | 14 |
| 14.39 ± 0.20 | 6.150 ± 0.085 | 13 |
| 15.41 ± 0.20 | 5.745 ± 0.074 | 13 |
| 16.01 ± 0.20 | 5.531 ± 0.069 | 30 |
| 16.43 ± 0.20 | 5.391 ± 0.065 | 10 |
| 16.74 ± 0.20 | 5.292 ± 0.063 | 13 |
| 17.01 ± 0.20 | 5.208 ± 0.061 | 30 |
| 17.31 ± 0.20 | 5.119 ± 0.059 | 25 |
| 17.63 ± 0.20 | 5.027 ± 0.057 | 78 |
| 18.17 ± 0.20 | 4.878 ± 0.053 | 23 |
| 19.01 ± 0.20 | 4.665 ± 0.049 | 11 |
| 19.26 ± 0.20 | 4.605 ± 0.047 | 16 |
| 19.51 ± 0.20 | 4.546 ± 0.046 | 9 |
| 20.08 ± 0.20 | 4.419 ± 0.044 | 7 |
| 20.88 ± 0.20 | 4.251 ± 0.040 | 10 |
| 21.36 ± 0.20 | 4.156 ± 0.038 | 28 |
| 22.35 ± 0.20 | 3.975 ± 0.035 | 20 |
| 23.03 ± 0.20 | 3.859 ± 0.033 | 35 |
| 24.30 ± 0.20 | 3.660 ± 0.030 | 12 |
| 25.65 ± 0.20 | 3.470 ± 0.027 | 13 |
| 25.79 ± 0.20 | 3.452 ± 0.026 | 11 |
| 26.42 ± 0.20 | 3.371 ± 0.025 | 7 |
| 27.42 ± 0.20 | 3.250 ± 0.023 | 7 |
| 28.00 ± 0.20 | 3.184 ± 0.022 | 6 |

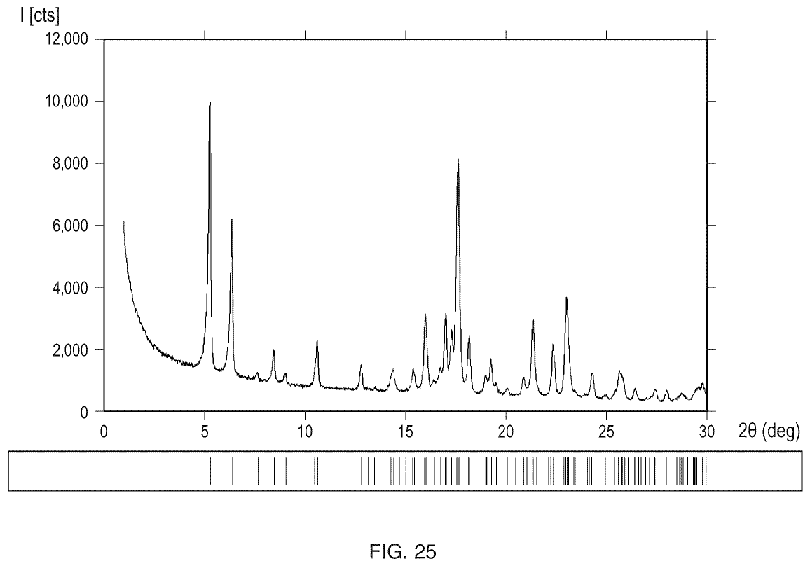

The XRPD pattern for the anhydrous crystalline citrate salt of the compound of formula (I) was successfully indexed (FIG. 25). The volume from the unit cell was consistent with an anhydrous mono-citrate salt (Table 23).

TABLE 23
Unit Cell Parameters of the Anhydrous Crystalline
Citrate Salt of the Compound of Formula (I)

| | Primitive |
|---|---|
| Bravais Type | Orthorhombic |
| a [Å] | 6.297 |
| b [Å] | 20.855 |
| c [Å] | 27.665 |
| α [deg] | 90 |
| β [deg] | 90 |
| γ [deg] | 90 |
| Volume [Å³/cell] | 3633.1 |
| Chiral Contents? | Chiral |
| Extinction Symbol | P 2₁ 2₁ 2₁ |
| Space Group(s) | P2₁2₁2₁ (19) |

(iii) Aqueous Solubility of the Anhydrous Crystalline Citrate Salt

The aqueous solubility of the anhydrous crystalline citrate salt of the compound of formula (I) at 25° C. was determined to be >111 mg/ml by solvent addition (same solubility determination method described in Example 2).

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

We claim:

1. A crystalline form of a compound of formula (I):

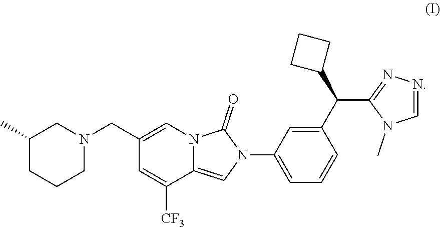 [SMILES: C[C@H]1CCCN(Cc2cc(C(F)(F)F)c3cn(-c4cccc([C@H](c5nncn5C)C5CCC5)c4)c(=O)n3c2)C1]

2. The crystalline form of claim 1, wherein the crystalline form is a crystalline salt.

3. The crystalline form of claim 1 or 2, wherein the crystalline form is an unsolvated crystalline form.

4. The crystalline form of claim 1 or 2, wherein the crystalline form is a crystalline solvate.

5. A crystalline form of a compound of formula (I): 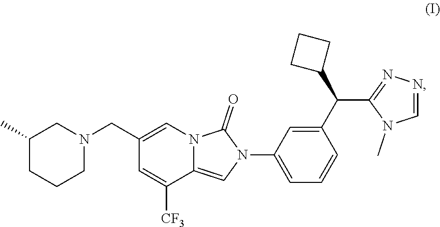 [SMILES: C[C@H]1CCCN(Cc2cc(C(F)(F)F)c3cn(-c4cccc([C@H](c5nncn5C)C5CCC5)c4)c(=O)n3c2)C1]
wherein the crystalline form is characterized by an X-ray powder diffraction (XRPD) pattern comprising one or more peaks selected from 6.0°±0.2°, 8.6°±0.2°, 14.3°±0.2°, and 16.3°±0.2°2-theta.

6. The crystalline form of claim 5, wherein the XRPD pattern further comprises one or more peaks selected from 15.6°±0.2°, 17.4°±0.2°, 18.2°±0.2°, 19.9°±0.2°, 20.4°±0.2°, and 21.5°±0.2°2-theta.

7. The crystalline form of claim 5 or 6, wherein the XRPD pattern further comprises one or more peaks selected from 7.1°±0.2°, 11.7°±0.2°, 12.1°±0.2°, 14.7°±0.2°, 15.1°±0.2°, 18.5°±0.20, 19.6°±0.2°, 20.6°±0.2°, 20.9°±0.2°, 22.0°±0.2°, 22.3°±0.2°, 22.7°±0.2°, 23.00±0.20, 23.2°±0.2°, 24.4°±0.2°, 24.8°±0.2°, 25.2°±0.2°, 25.6°±0.2°, 26.1°±0.2°, 26.40±0.2°, 27.1°±0.2°, 27.5°±0.2°, 28.1°±0.2°, 28.5°±0.2°, 28.7°±0.2°, 29.3°±0.2°, 29.8°±0.2°, and 30.5°±0.2°2-theta.

8. The crystalline form of any one of claims 5-7, wherein the crystalline form is characterized by an XRPD pattern substantially the same as shown in FIG. 1.

9. The crystalline form of any one of claims 5-8, wherein the crystalline form exists in an orthorhombic crystal system and has a P2₁2₁2₁ space group.

10. The crystalline form of claim 9, wherein the crystalline form is characterized by the following crystallographic unit cell parameters:

| | | |
|---|---|---|
| Unit cell dimensions | a = 6.37450(10) Å | α = 90° |
| | b = 18.1063(3) Å | β = 90° |
| | c = 24.8603(4) Å | γ = 90° |
| | |
|---|---|
| Volume | 2869.34(8) Å³ |

11. The crystalline form of any one of claims 5-10, the crystalline form is characterized by an endotherm with a peak onset of about 165° C. to about 180° C., as determined by differential scanning calorimetry.

12. The crystalline form of any one of claims 5-11, wherein the crystalline form is an anhydrous crystalline form.

13. A crystalline solvate of a compound of formula (I):

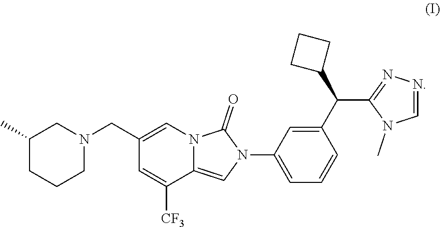 [SMILES: C[C@H]1CCCN(Cc2cc(C(F)(F)F)c3cn(-c4cccc([C@H](c5nncn5C)C5CCC5)c4)c(=O)n3c2)C1]

14. The crystalline solvate of claim 13, wherein the crystalline solvate is an acetone solvate, a p-dioxanes solvate, or a tetrahydrofuran solvate.

15. A crystalline acetone solvate of a compound of formula (I):

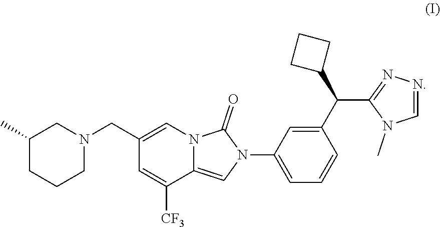 [SMILES: C[C@H]1CCCN(Cc2cc(C(F)(F)F)c3cn(-c4cccc([C@H](c5nncn5C)C5CCC5)c4)c(=O)n3c2)C1]

16. The crystalline acetone solvate of claim 15, wherein the acetone solvate is a mono-acetone solvate.

17. The crystalline acetone solvate of claim 15 or 16, wherein the crystalline acetone solvate is characterized by an X-ray powder diffraction (XRPD) pattern comprising one or more peaks selected from 6.4°±0.2°, 16.2°±0.2°, 17.2°±0.2°, and 22.0°±0.2°2-theta.

18. The crystalline acetone solvate of claim 17, wherein the XRPD pattern further comprises one or more peaks selected from 12.0°±0.2°, 14.3°±0.2°, 15.7°±0.2°, 17.8°±0.2°, 20.2°±0.2°, 21.8°±0.2°, and 22.6°±0.2°2-theta.

19. The crystalline acetone solvate of claim 17 or 18, wherein the XRPD pattern further comprises one or more peaks selected from 7.8°±0.2°, 9.8°±0.2°, 10.4°±0.2°, 12.8°±0.2°, 14.1°±0.2°, 15.1°±0.2°, 17.6°±0.2°, 18.4°±0.2°, 18.6°±0.2°, 19.2°±0.2°, 20.5°±0.20, 21.2°±0.2°, 22.8°±0.2°, 23.3°±0.2°, 23.6°±0.2°, 24.0°±0.2°, 24.3°±0.2°, 24.9°±0.20, 25.0°±0.2°, 25.4°±0.2°, 25.8°±0.2°, 26.1°±0.2°, 26.3°±0.2°, 27.0°±0.2°, 27.7°±0.20, 28.3°±0.2°, 28.7°±0.2°, 29.3°±0.2°, 29.7°±0.2°, 30.1°±0.2°, 30.6°±0.20, and 31.1°±0.20 2-theta.

20. The crystalline acetone solvate of any one of claims 17-19, wherein the crystalline acetone solvate is characterized by an XRPD pattern substantially the same as shown in FIG. 6.

21. The crystalline acetone solvate of any one of claims 15-20, wherein the crystalline acetone solvate exists in an orthorhombic crystal system and has a P2₁2₁2₁ space group.

22. The crystalline acetone solvate of claim 21, wherein the crystalline acetone solvate is characterized by the following crystallographic unit cell parameters:

| | | |
|---|---|---|
| Unit cell dimensions | a = 6.251 Å | α = 90° |
| | b = 15.082 Å | β = 90° |
| | c = 33.920 Å | γ = 90° |
| | |
|---|---|
| Volume | 3197.9 Å³ |

23. The crystalline acetone solvate of any one of claims 15-22, wherein the crystalline acetone solvate is characterized by one or more endotherms with peak maxima selected from about 100° C., about 107° C., and about 173° C., as determined by differential scanning calorimetry.

24. A crystalline p-dioxane solvate of a compound of formula (I):

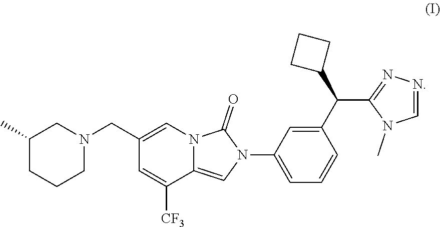 [SMILES: C[C@H]1CCCN(Cc2cc(C(F)(F)F)c3cn(-c4cccc([C@H](c5nncn5C)C5CCC5)c4)c(=O)n3c2)C1]

25. The crystalline p-dioxane solvate of claim 24, wherein the p-dioxane solvate is a mono-p-dioxane solvate.

26. The crystalline p-dioxane solvate of claim 24 or 25, wherein the crystalline p-dioxane solvate is characterized by an X-ray powder diffraction (XRPD) pattern comprising one or more peaks selected from 6.3°±0.2°, 16.0°±0.2°, 17.0°±0.2°, and 21.8°±0.2°2-theta.

27. The crystalline p-dioxane solvate of claim 26, wherein the XRPD pattern further comprises one or more peaks selected from 11.8°±0.2°, 14.2°±0.2°, 17.5°±0.2°, 20.0°±0.2°, and 21.6°±0.2°2-theta.

28. The crystalline p-dioxane solvate of claim 26 or 27, wherein the XRPD pattern further comprises one or more peaks selected from 5.2°, 0.2°, 7.7°±0.2°, 10.3°±0.2°, 12.6°±0.2° 14.9°±0.2°, 15.4°±0.2°, 15.5°±0.2°, 18.1°±0.2°, 18.4°±0.2°, 18.9°±0.2°, 19.1°±0.20, 19.4°±0.2°, 20.2°±0.2°, 21.0°±0.2°, 22.4°±0.2°, 23.0°±0.2°, 23.1°±0.2°, 23.8°±0.20, 24.0°±0.2°, 24.7°±0.2°, 25.2°±0.2°, 25.4°±0.2°, 25.8°±0.2°, 27.3°±0.2°, 27.8°±0.20, 28.0°±0.2°, 28.3°±0.2°, and 28.9°±0.2°2-theta.

29. The crystalline p-dioxane solvate of any one of claims 26-28, wherein the crystalline p-dioxane solvate is characterized by an XRPD pattern substantially the same as shown in FIG. 10.

30. The crystalline p-dioxane solvate of any one of claims 24-29, wherein the crystalline p-dioxane solvate exists in an orthorhombic crystal system and has a P2₁2₁2₁ space group.

31. The crystalline p-dioxane solvate of claim 30, wherein the crystalline p-dioxane solvate is characterized by the following crystallographic unit cell parameters:

| | | |
|---|---|---|
| Unit cell dimensions | a = 6.316 Å | α = 90° |
| | b = 15.364 Å | β = 90° |
| | c = 34.166 Å | γ = 90° |
| | |
|---|---|
| Volume | 3315.4 Å³ |

32. The crystalline p-dioxane solvate of any one of claims 24-31, wherein the crystalline p-dioxane solvate is characterized by an endotherm with a peak onset of about 94° C., as determined by differential scanning calorimetry.

33. A crystalline tetrahydrofuran solvate of a compound of formula (I):

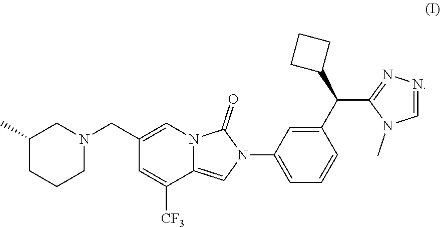 [SMILES: C[C@H]1CCCN(Cc2cc(C(F)(F)F)c3cn(-c4cccc([C@H](c5nncn5C)C5CCC5)c4)c(=O)n3c2)C1]

34. The crystalline tetrahydrofuran solvate of claim 33, wherein the tetrahydrofuran solvate is a mono-tetrahydrofuran solvate.

35. The crystalline tetrahydrofuran solvate of claim 33 or 34, wherein the crystalline tetrahydrofuran solvate is characterized by an X-ray powder diffraction (XRPD) pattern comprising one or more peaks selected from 6.3°±0.2°, 16.1°±0.2°, 17.3°±0.2°, and 22.9°±0.2°2-theta.

36. The crystalline tetrahydrofuran solvate of claim 35, wherein the XRPD pattern further comprises one or more peaks selected from 17.1°±0.2°, 17.9°±0.2°, and 22.4°±0.2°2-theta.

37. The crystalline tetrahydrofuran solvate of claim 35 or 36, wherein the XRPD pattern further comprises one or more peaks selected from 7.7°±0.2°, 11.4°±0.2°, 11.7°±0.2°, 11.9°±0.2°, 12.5°±0.2°, 14.3°±0.2°, 15.2°±0.2°, 15.5°±0.2°, 18.3°±0.2°, 18.9°±0.2°, 19.8°±0.2°, 20.1°±0.2°, 21.0°±0.2°, 21.7°±0.2°, 21.9°±0.2°, 23.5°±0.2°, 24.1°±0.2°, 24.6°±0.2°, 25.3°±0.2°, 25.9°±0.2°, 27.1°±0.2°, 27.3°±0.2°, 28.4°±0.2°, 28.9°±0.2°, 29.5°±0.2°, 30.0°±0.2°, and 30.8°±0.2°2-theta.

38. The crystalline tetrahydrofuran solvate of any one of claims 35-37, wherein the crystalline tetrahydrofuran solvate is characterized by an XRPD pattern substantially the same as shown in FIG. 14.

39. The crystalline tetrahydrofuran solvate of any one of claims 33-38, wherein the crystalline tetrahydrofuran solvate exists in an orthorhombic crystal system and has a P2₁2₁2₁ space group.

40. The crystalline tetrahydrofuran solvate of claim 39, wherein the crystalline tetrahydrofuran solvate is characterized by the following crystallographic unit cell parameters:

| | | |
|---|---|---|
| Unit cell dimensions | a = 6.289 Å | α = 90° |
| | b = 15.515 Å | β = 90° |
| | c = 33.823 Å | γ = 90° |
| | |
|---|---|
| Volume | 3300.2 Å³ |

41. The crystalline tetrahydrofuran solvate of any one of claims 33-40, wherein the crystalline tetrahydrofuran solvate is characterized by an endotherm with a peak onset of about 85° C., as determined by differential scanning calorimetry.

42. A crystalline citrate salt of a compound of formula (I):

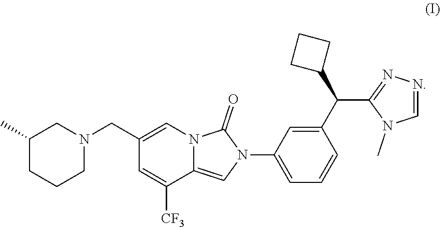 [SMILES: C[C@H]1CCCN(Cc2cc(C(F)(F)F)c3cn(-c4cccc([C@H](c5nncn5C)C5CCC5)c4)c(=O)n3c2)C1]

43. The crystalline citrate salt of claim 42, wherein the crystalline citrate salt is characterized by an X-ray powder diffraction (XRPD) pattern comprising one or more peaks selected from 5.0±0.2°, 6.6±0.2, 17.6±0.2°, and 18.2±0.2°2-theta.

44. The crystalline citrate salt of claim 43, wherein the XRPD pattern further comprises one or more peaks selected from 15.°±0.2°, 15.3±0.2°, 16.9±0.2, 19.7±0.2, 20.1±0.2°, 22.6±0.2°, 22.8±0.2°, and 24.8±0.2°2-theta.

45. The crystalline citrate salt of claim 43 or 44, wherein the XRPD pattern further comprises one or more peaks selected from 8.6±0.2, 10.°±0.2°, 11.°±0.2, 11.5±0.2°, 13.2±0.2, 13.3±0.2°, 14.6±0.2°, 15.9±0.2°, 16.3±0.2°, 16.5±0.2°, 21.0 0.2°, 21.5±0.2°, 21.8±0.2°, 23.2±0.2°, 23.4±0.2°, 23.9±0.2°, 24.6±0.2°, 25.2±0.2°, 26.°±0.2°, 26.6±0.2°, 27.3±0.2°, 28.9±0.2°, 29.5±0.2, 29.8±0.2, and 30.4±0.2°2-theta.

46. The crystalline citrate salt of any one of claims 43-45, wherein the crystalline citrate salt is characterized by an XRPD pattern substantially the same as shown in FIG. 18.

47. The crystalline citrate salt of any one of claims 42-46, wherein the crystalline citrate salt exists in an orthorhombic crystal system and has a P2₁2₁2₁ space group.

48. The crystalline citrate salt of claim 47, wherein the crystalline citrate salt is characterized by the following crystallographic unit cell parameters:

| | | |
|---|---|---|
| Unit cell dimensions | a = 6.150 Å | α = 90° |
| | b = 20.489 Å | β = 90° |
| | c = 34.845 Å | γ = 90° |
| | |
|---|---|
| Volume | 4390.7 Å³ |

49. The crystalline citrate salt of any one of claims 42-48, wherein the crystalline citrate salt is characterized by an endotherm with a peak onset of about 110° C., as determined by differential scanning calorimetry.

50. The crystalline citrate salt of any one of claims 42-49, wherein the crystalline citrate salt is an acetone solvated crystalline citrate salt.

51. The crystalline citrate salt of claim 42, wherein the crystalline citrate salt is characterized by an XRPD pattern comprising one or more peaks selected from 5.1±0.2°, 6.9±0.2, 17.8±0.2°, and 18.7±0.2°2-theta.

52. The crystalline citrate salt of claim 51, wherein the XRPD pattern further comprises one or more peaks selected from 17.2±0.2°, 22.3±0.2°, 23.3±0.2, and 23.6±0.2°2-theta.

53. The crystalline citrate salt of claim 51 or 52, wherein the XRPD pattern further comprises one or more peaks selected from 5.4±0.2°, 8.6±0.2°, 9.2±0.2°, 10.1±0.2°, 10.8±0.2°, 11.7±0.2°, 13.2±0.2°, 13.9±0.2°, 14.0 0.2°, 14.8±0.2°, 15.2±0.2°, 15.4±0.2°, 16.1±0.2°, 16.9±0.2°, 17.4±0.2°, 18.°±0.2°, 19.1±0.2°, 19.5±0.2°, 19.7±0.2°, 19.9±0.2°, 20.2±0.2°, 20.4±0.2°, 20.9±0.2°, 21.1±0.2°, 21.8±0.2°, 21.9±0.2°, 22.6±0.2°, 22.8±0.2°, 23.8±0.2°, 24.1±0.2°, 24.4±0.2, 25.2±0.2°, 25.5±0.2°, 26.°±0.2°, 26.5±0.2°, 26.7±0.2°, 27.4±0.2, 27.9±0.2, 28.4±0.2°2-theta.

54. The crystalline citrate salt of any one of claims 51-53, wherein the crystalline citrate salt is characterized by an XRPD pattern substantially the same as shown in FIG. 22.

55. The crystalline citrate salt of any one of claims 42 and 51-54, wherein the crystalline citrate salt exists in an orthorhombic crystal system and has a P2₁2₁2₁ space group.

56. The crystalline citrate salt of claim 55, wherein the crystalline citrate salt is characterized by the following crystallographic unit cell parameters:

| | | |
|---|---|---|
| Unit cell dimensions | a = 6.081 Å | α = 90° |
| | b = 20.605 Å | β = 90° |
| | c = 32.628 Å | γ = 90° |
| | |
|---|---|
| Volume | 4088.3 Å³ |

57. The crystalline citrate salt of any one of claims 42 and 51-56, wherein the crystalline citrate salt is an acetonitrile solvated crystalline citrate salt.

58. The crystalline citrate salt of claim 42, wherein the crystalline citrate salt is characterized by an XRPD pattern comprising one or more peaks selected from 5.3±0.2°, 6.4±0.2, 17.6±0.2°, and 23.°±0.2°2-theta.

59. The crystalline citrate salt of claim 58, wherein the XRPD pattern further comprises one or more peaks selected from 8.5±0.2°, 10.6±0.2°, 16.°±0.2, 17.°±0.2, 17.3±0.2°, 18.2±0.2°, 21.4±0.2°, and 22.4±0.2°2-theta.

60. The crystalline citrate salt of claim 58 or 59, wherein the XRPD pattern further comprises one or more peaks selected from 7.6±0.2, 9.1±0.2, 12.8±0.2°, 14.4±0.2°, 15.4±0.2, 16.4±0.2°, 16.7±0.2°, 19.°±0.2°, 19.3±0.2°, 19.5±0.2°, 20.1±0.2°, 20.9±0.2°, 24.3±0.2°, 25.7±0.2, 25.8±0.2, 26.4±0.2°, 27.4±0.2°, and 28.°±0.2°2-theta.

61. The crystalline citrate salt of any one of claims 58-60, wherein the crystalline citrate salt is characterized by an XRPD pattern substantially the same as shown in FIG. 24.

62. The crystalline citrate salt of any one of claims 42 and 58-61, wherein the crystalline citrate salt exists in an orthorhombic crystal system and has a P2₁2₁2₁ space group.

63. The crystalline citrate salt of claim 62, wherein the crystalline citrate salt is characterized by the following crystallographic unit cell parameters:

| | | |
|---|---|---|
| Unit cell dimensions | a = 6.297 Å | α = 90° |
| | b = 20.855 Å | β = 90° |
| | c = 27.665 Å | γ = 90° |
| | |
|---|---|
| Volume | 3633.1 Å³ |

64. The crystalline citrate salt of any one of claims 42 and 58-63, wherein the crystalline citrate salt is characterized by an endotherm with a peak maximum of about 131° C., as determined by differential scanning calorimetry.

65. The crystalline citrate salt of any one of claims 42 and 58-64, wherein the crystalline citrate salt is an anhydrous citrate salt.

66. The crystalline citrate salt of any one of claims 42-65, wherein the citrate salt is a mono-citrate salt.

67. A pharmaceutical composition comprising:
the crystalline form of any one of claims 1-12, the crystalline solvate of any one of claims 13-41, or the crystalline citrate salt of any one of claims 42-66; and
a pharmaceutically acceptable excipient.

68. A method of treating a disease or condition associated with cell proliferation comprising administering a therapeutically effective amount of a crystalline form of any one of claims 1-12, a crystalline solvate of any one of claims 13-41, a crystalline citrate salt of any one of claims 42-66, or a pharmaceutical composition of claim 67 to a subject in need thereof.

69. The method of claim 68, wherein the disease or condition associated with cell proliferation is hyperplasia or a cancer.

70. The method of claim 69, wherein the cancer is a hematologic cancer.

71. The method of claim 70, wherein the hematologic cancer is selected from a group consisting of a lymphoma, a leukemia, and a myeloma.

72. The method of claim 69, wherein the cancer is a non-hematologic cancer.

73. The method of claim 72, wherein the non-hematologic cancer is a sarcoma or a carcinoma.

74. The method of any one of claims 68-73, wherein the subject has one or more of increased T-cell activation, increased T-cell proliferation, decreased T-cell exhaustion, decreased T-cell anergy, and decreased T-cell tolerance after administration of a crystalline form of any one of claims 1-12, a crystalline solvate of any one of claims 13-41, a crystalline citrate salt of any one of claims 42-66, or a pharmaceutical composition of claim 67.

75. The method of claim 74, wherein increased T-cell activation comprises increased production of a cytokines.

76. The method of any one of claims 68-73, wherein the subject has increased NK-cell activation.
